SEQUENCE LISTING (58 sequences)

SEQ ID NO: 1 - protein, 736 aa, Artificial Sequence
  Description of Artificial Sequence Synthetic polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 736 aa, Artificial Sequence
  Description of Artificial Sequence Synthetic polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEAPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPVGTRYLTRPL

SEQ ID NO: 3 - protein, 736 aa, Artificial Sequence
  Description of Artificial Sequence Synthetic polypeptide
MAADGYLPDWLEDNLSEGIREWWDLKPGAPKPKANQQKQDDGRGLVLPGYKYLGPFNGLDKGEPVSAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKERLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 4 - protein, 736 aa, Artificial Sequence
  Description of Artificial Sequence Synthetic polypeptide
MAADGYLPDWLEDNLSEGIREWWDLKPGAPKPKANQQEQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHATGTLPGMVWQDRDVYLQGPIWAKIPHTDGHFHPSPLMGGFGLKNPPPQILIKTTPVPANPPAEFSATKFASFITQYSTGQVSVEIEWELQKENSKRWNPEVQYTSNYAKSANVDFTVDDNGLYTEPRPIGTRYLTRPL

SEQ ID NO: 5 - protein, 735 aa, Artificial Sequence [sequence omitted]

SEQ ID NO: 6 - protein, 735 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQEGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 7 - protein, 737 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSSGGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 8 - protein, 737 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQGTSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 9 - protein, 735 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTQTTGGTANTQTLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 10 - protein, 734 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTINGSGQNQQTLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 11 - protein, 735 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTQTTGGTTNTQTLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 12 - protein, 736 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTINGSGQNQQTLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 13 - protein, 734 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTQTTGTGGTQTLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 14 - protein, 734 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 15 - protein, 731 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTSGNTANPTQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 16 - protein, 736 aa, Artificial Sequence
  Description of Artificial Sequence Synthetic polypeptide
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 17 - protein, 736 aa, Artificial Sequence
  Description of Artificial Sequence Synthetic polypeptide
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEGAAKTAPGKKRPVDQSPQEPDSSSGVGKSGKQLARKRLNFGQTGDSESVPDPQPLGEPPAAPTSLGSNTMASGGGAPMADNNEGADGVGNSSGNWHCDSQWLGDRVITTSTRTWALPTYNSHLYKQISGQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 18 - protein, 736 aa, Artificial Sequence
  Description of Artificial Sequence Synthetic polypeptide
MAADGYLPDWLEDNLSEGIREWWALKPGVPQPKANQQHQDDRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATVQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 19 - protein, 736 aa, Artificial Sequence
  Description of Artificial Sequence Synthetic polypeptide
MAADGYLPDWLEDNLSEGIREWWALKPGVPQPKANQQHQDNRRGLVLPGYKYLGPGNGLDEGEPVNEADAAALEHDKAYDQQLKAGDNPYLKYNHADAEFQERLQEDTSFGGNLGRAVFQAKKRILEPLGLVEGAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNSHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 20 - protein, 736 aa, Artificial Sequence
  Description of Artificial Sequence Synthetic polypeptide
MAADGYLPDWLEDNLSEGIREWWALKPGVPQPKANQQHQDDRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGNRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 21 - protein, 734 aa, Artificial Sequence
  Description of Artificial Sequence Synthetic polypeptide
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 22 - protein, 724 aa, Artificial Sequence
  Description of Artificial Sequence Synthetic polypeptide
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 23 - protein, 724 aa, Artificial Sequence
  Description of Artificial Sequence Synthetic polypeptide
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGSMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 24 - protein, 724 aa, Artificial Sequence
  Description of Artificial Sequence Synthetic polypeptide
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTHKNWFPGPMGRTQGWNLGSGVNRASVSAFATTNRMELEGASYQVPPQSNGMTNNLRGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 25 - protein, 724 aa, Artificial Sequence
  Description of Artificial Sequence Synthetic polypeptide
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFAATNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGGVWMERDVYLQGPIWAKIPETGAHFHPSPAMGGFGLKHPPPMMLIKNTPVPGNTTSFSDVPVSSFITQYSTGQVTVEMEWELKKENSKRWNPEIQYTNNYNDPQFVDFAPDSTGEYRTTRPIGTRYLTRPL

SEQ ID NO: 26 - protein, 724 aa, Artificial Sequence
  Description of Artificial Sequence Synthetic polypeptide
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKVVFQAKKRVLEPFGLVEEGAKTAPTGKRIDDHFPKRKKARTEEDSKPSTSSDAEAGPSGSQQLQIPAQPASSLGADTMSAGGGGPLGGNNQGADGVGNASGDWHCDSTWMGDRVVTKSTRTWVLPSYNNHQYREIKSGSVDGSNANAYFGYSTPWGYFDFNRFHSHWGPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSALATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 27 - protein, 724 aa, Artificial Sequence
  Description of Artificial Sequence Synthetic polypeptide
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLAEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRAKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNLEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVVHNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 28 - protein, 736 aa, Artificial Sequence
  Description of Artificial Sequence Synthetic polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 29 - protein, 736 aa, Artificial Sequence
  Description of Artificial Sequence Synthetic polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 30 - protein, 736 aa, Artificial Sequence
  Description of Artificial Sequence Synthetic polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 31 - protein, 736 aa, Artificial Sequence
  Description of Artificial Sequence Synthetic polypeptide
MAADGYFPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGRTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSPDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYSEPRPIGTRYLTRPL

SEQ ID NO: 32 - protein, 736 aa, Artificial Sequence
  Description of Artificial Sequence Synthetic polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQTSSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDYNNSNFTWTGASKYNLNGRESIINPGTAMASHKDDEDKFFPMSGVMIFGKESAGASNTALDNVMITDEEEIKATNPVATERFGTVAVNFQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 33 - protein, 736 aa, Artificial Sequence
  Description of Artificial Sequence Synthetic polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHVMGALPGMVWQDRDVYLQGPIWAKIPHTDGHSHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 34 - protein, 737 aa, Artificial Sequence
  Description of Artificial Sequence Synthetic polypeptide
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 35 - protein, 738 aa, Artificial Sequence
  Description of Artificial Sequence Synthetic polypeptide
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 36 - protein, 736 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 37 - protein, 736 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVSAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVSDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPSPADVFMIPQYGYLTLNDGSQAVGRSSFYCLEYFPSQMLRTGNNFQFSYEFENVPFHSSYAHSQSLDRLMNPLIDQYLHYLSKTINGSGQNQQTLKFSVAGPSNMAVQGRNYIPGPSYRQQRVSTTVTQNNNSEFAWPGASSWALNGRNSLMNPGPAMASYKEGEDSFFPLSGSLIFGKQGTGRDDVDADKVIITNEEEIKTTNPVATESYGQVATNHQSAQAQAQTGWVQNQGILPGMVWQDRDVYLQGPIWAKIPHTDGNFHPSPLMGGFGMKHPPPQTLIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 38 - protein, 736 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLHYLSKTINGSGQNQQTLKFSVAGPSNMAVQGRNYIPGPSYRQQRVSTTVTQNNNSEFAWPGASSWALNGRNSLMNPGPAMASYKEGEDSFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQTLIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 39 - protein, 736 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGPVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQAATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 40 - protein, 736 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFGYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWDPEIQYTSNYYKSNNVEFAVNTEGVYSEPRPIGTRYLTRNL

SEQ ID NO: 41 - protein, 736 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNTAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 42 - protein, 736 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNLYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVVDNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 43 - protein, 736 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKPNNVEFAVNTEGVYSEPRPIGTRYLTRNL

SEQ ID NO: 44 - protein, 736 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQTKKRLLEPLGLVEEAAKTAPGKKRPVEQSPQEPGSSAGIGKSGAQPAKKRLNFGQTGDTESVPDPQPIGEPPAAPSGVGSLTMASGGGAPVADNNEGADGVGSSSGNWHCDSQWLGDRVIITSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLHYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 45 - protein, 736 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFRLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 46 - protein, 736 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALVHDKAYDQQLKAGDNPYLKCNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYLGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFTIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGPRYLTRNF

SEQ ID NO: 47 - protein, 736 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYLGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 48 - protein, 736 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLTNNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYIPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGVKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 49 - protein, 733 aa, Artificial Sequence
  Description of Artificial Sequence Synthetic polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 50 - protein, 736 aa, Artificial Sequence
  Description of Artificial Sequence Synthetic polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 51 - protein, 738 aa, Artificial Sequence
  Description of Artificial Sequence Synthetic polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 52 - protein, 738 aa, Artificial Sequence
  Description of Artificial Sequence Synthetic polypeptide
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 53 - protein, 736 aa, Artificial Sequence
  Description of Artificial Sequence Synthetic polypeptide
MAADGYLPDWLEDNLSEGIREWWDLKPGAPKPKANQQKQDDGRGLVLPGYKYLGPFNGLDKGEPVNAADAAALEHDKAYDQQLEAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPVTGSCFWQQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 54 - protein, 737 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 55 - protein, 737 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQQGNTQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 56 - protein, 727 aa, Artificial Sequence
  Description of Artificial Sequence Synthetic polypeptide
MSFVDHPPDWLEEVGEGLHEFLELEAGPPKPKPNQQKQDNARGLVLPGYNYLGPFNGLDKGEPVNRADAVAREHDISYNEQLQAGDNPYLKYNHADAEFQEKLKDDTSFGGNLGKAIFQAKKRVLEPFGLVEAPVKTAPAKKRPIEKSPAEPSSSKGIGKAGQQPARKRLNFGQTGDTDSAADPQPLGEPPAAPSGLGTGTMASGGGAPMADNNEGADGVGNASGNWHCDSTWLGDRVITTSTRTWALPTYNNHLYKQISSSNYGANNDNHYFGYSTPWGYFDFNRFHCHFSPRDWQRLINNNWGFRPKRLNFKLFNIQVKEVTETDGTKTIANNLTSTVQVFADSEYQLPYVLGSAHQGCFPPFPADVFMIPQYGYLTLNNGSQAMGRSSFYCLEYFPSQMLRTGNNFTFSYTFEDVPFHSSYAHSQSLDRLMNPLIDQYLYYLSKTAGGLAFSQAGPTTMRNQSRNWLPGPCFRQQRVSERSTENNNGDFSWTGTTRYHLNGRDSAMNPGPAMASHKDDEDRFFPQNGVLIFGTPNATASNAPLENVLITDEEEIRTTNPVATEEYGIVANNKQDSSTQATTAIVNAQGILPGMVWQDRDVYLQGPIWAKIPHTDGHFHPSPLMGGFGLKHPPPQILIKNTPVPSNPPEKFTQEKLNAFITQYSTGQVSVEIEWELQKENSKRWNPEVQYTSNYNKSVNVDFTVDTNGMYSEPRTIGTRYLTRNL

SEQ ID NO: 57 - protein, 713 aa, Artificial Sequence
  Description of Artificial Sequence Synthetic polypeptide
MSFFDWLGKQYAQGAAEFWDLKSGPPAPKKARKDGSAGFNFPGHKYLGPGNSLDRGDPVDADDAAAQKHDQSYQEQLEAGDNPYLKYNHADREFQEALKDDTSFEGNLARGLFEAKKLVAEPLGLVEPELAPPSGRKRPVQSSQESGYSSSQDKRPNLDVDEEDREFAAAAAETETGSAPPTGNLGPGTMAGGGSAPIDDGSYGADGVGNASGDWHCDSTWLDNCVITRTTRTWNLPTYNNHIYKRLNGTTSGDQSYFGFSTPWGYFDFNRFHCHFSPRDWQRLINNNWGLRPKSLRFKIFNIQVKEVTTQDSTKIISNNLTSTVQVFADTEYQLPYVIGSAHEGCLPPFPADVFMLPQYGYCTRQDGNSNNPTPRSAFYCLEYFPSKMLRTGNSFEFTYNFEKVPFHSMWAHNQSLDRLMNPLIDQYLYYLDVTSSTGFTYQKGVHTNLPEQERNWLPGPGIRNQAWFNSATGNNPLTGTWQYSNKYVLENRASKIAPGPAMGIESTKFDGNGIIFSKEYITNVNTANPNQVNITRETEINSTNPLAGGSLGAHANNSQNTTTAPTLDHTNVMGVFPGSVWQDRDIYLQGQIWAKIPHTDGHFHPSPLMGGFGLKNPPPQILIKNTPVPADPPTEFNANKISSFITQYSTGQVTVEMEWELQKETSKRWNPEIQYSDDSSSTSGSILHFAPDDVGNYKEFRSIGTRYLTRPL

SEQ ID NO: 58 - protein, 738 aa, Artificial Sequence
  Description of Artificial Sequence Synthetic polypeptide
MSLISDAIPDWLERLVKKGVNAAADFYHLESGPPHPKANQQTQESPEKDDSRGLVFPGYKYLGPFNGLDKGKPVNEADAAALEHDKAYDLELKDGHNPYFEYNEADRRFQERLKDDTSFGGNLGKAIFQAKKRVLEPFGLIEQPDNTAGTGEKRPERVDDFFPKKKKAKTEQGKAPAQTGEDPGEGTSSNAGSSAPSSVGSSVMAEGGGGPMGDAGQGADGVGNSSGNWHCDSQWLDNGVVTRTTRTWVLPSYNNHLYKRIQGPGGTDPNNKFFGFSTPWGYFDYNRFHCHFSPRDWQRLINNNWGIRPKAMRFRLFNIQVKEVTVQDSNTTIANNLTSTVQVFADKDYQLPYVLGSATEGTFPPFPADIYTIPQYGYCTLNYNNEAVDRSAFYCLDYFPSDMLRTGNNFEFTYTFEDVPFHSMFAHNQTLDRLMNPLVDQYLWAFSSVSQTGSSGRALNYSRATKTNMATQYRNWLPGPFVRDQQIFTGASNITQNNVFNVWDKGKQWVIDNRINMMQPGPAAATTFSGEPDRQAMQNTLAFSRTVYDQTTSTTDRNQLLITNEDEIRPTNSVGIDTWGVVPNNNQSKVTAGTRAAINNQGALPGMVWQNRDIYLQGPIWAKIPDTDNHFHPSPLIGGFGCKHPPPQIFIKNTPVPANPSETFQTAKVASFINQYSTGQCTVEIFWELKKETSKRWNPEIQFTSNFGNAADIQFAVSDTGSYSEPRPIGTRYLTKPL

(19) United States
(12) Patent Application Publication
(10) Pub. No.: US 2021/0207167 A1
(43) Pub. Date: Jul. 8, 2021

(54) AAV SEROTYPES FOR BRAIN SPECIFIC PAYLOAD DELIVERY

(71) Applicant: VOYAGER THERAPEUTICS, INC., Cambridge, MA (US)

(72) Inventors: Jinzhao HOU, Lexington, MA (US); Kei ADACHI, Portland, OR (US)

(73) Assignee: Voyager Therapeutics, Inc., Cambridge, MA (US)

(21) Appl. No.: 17/055,888

(22) PCT Filed: May 16, 2019
(86) PCT No.: PCT/US2019/032560

§ 371 Date: Nov. 16, 2020

Related U.S. Application Data
(60) Provisional application No. 62/729,643, filed on Sep. 11, 2018.
(60) Provisional application No. 62/672,548, filed on May 16, 2018.

(51) Int. Cl. C12N 15/86; C07K 14/005; A61P 25/28; C12N 7/00; A61K 38/00

(52) U.S. Cl. CPC C12N 15/86; C07K 14/005; A61P 25/28; A61K 38/00; C12N 2750/14143; C12N 2750/14122; C12N 7/00

(57) ABSTRACT

The present disclosure relates to compositions, methods, and processes for the design, preparation, manufacture, use, and/or formulation of adeno-associated virus (AAV) particles for improved biodistribution and/or expression to particular regions of the central nervous system (CNS).

This application claims the benefit of U.S. Provisional Patent Application No. 62/672,548, entitled “Compositions and methods for delivery of AAV”, filed May 16, 2018, and U.S. Provisional Patent Application No. 62/729,643, entitled “Barcoding” filed Sep. 11, 2018, the contents of each of which are herein incorporated by reference in their entirety.

### REFERENCE TO THE SEQUENCE LISTING

The present application is being filed along with a Sequence Listing in electronic format as an ASCII text file. The Sequence Listing is provided as an ASCII text file entitled 2057_1020PCT_SL.txt, created on May 16, 2019, which is 370,197 bytes in size. The Sequence Listing is incorporated herein by reference in its entirety.

### FIELD OF THE DISCLOSURE

The present disclosure relates to compositions, methods, and processes for the design, preparation, manufacture, use, and/or formulation of adeno-associated virus (AAV) particles for improved biodistribution and/or expression to particular regions of the central nervous system (CNS).

### BACKGROUND

Adeno-associated viral (AAV) particles are a promising candidate for therapeutic gene delivery and have proven safe and efficacious in clinical trial.

However, delivery of AAV to some systems in the body has proven to be particularly challenging. One example of a body system where delivery is challenging is the central nervous system (CNS). Delivery of AAV to regions of the CNS has proven to be particularly challenging, requiring invasive surgeries for sufficient levels of gene transfer (See e.g., Bevan et al. Mol. Ther. 2011 November; 19(11): 1971-1980). There remains a need in the art for AAV particles that may be able to efficiently target regions critical for treating a multitude of CNS diseases.

To identify AAV capsid proteins with desired tropism profiles, libraries of novel capsids have been created and screened. A variety of capsid engineering methods have been used, including DNA barcoding, directed evolution, random peptide insertions, and capsid shuffling and/or chimeras. In one such method, known as AAV Barcode-seq (see Adachi K et al, Nature Communications 5:3075 (2014)), a series of unique DNA-barcodes was added to the viral vector genome of each member of an AAV library. The barcode served as a tool for the identification of the capsid after experimental analysis. The incorporation of the barcode enabled the identification of capsids with desired properties after screening, such as enhanced tropism for CNS tissues.

The present disclosure addresses the need for AAV particles to target regions of the CNS relevant to diseases and other indications by incorporating the AAV Barcode-seq method to identify AAV capsids with increased tropism to CNS tissues upon administration to the cerebrospinal fluid (CSF).

### SUMMARY

The details of various embodiments of the disclosure are set forth in the description below. Other features, objects, and advantages of the disclosure will be apparent from the description, drawings, and the claims. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

Non-limiting embodiments of the subject matter disclosed herein are presented below:

1. A method of delivering a payload molecule to at least one brain region of a subject, comprising administering at least one AAV particle to cerebrospinal fluid (CSF) of the subject, wherein the at least one AAV particle comprises a viral genome that encodes at least one payload molecule, and a capsid protein, whereby the at least one payload molecule is expressed in at least one brain region, and wherein the capsid protein serotype is selected from the group consisting of CLv-1, CLv-6, AAVCkd-7, AAV2-R585E, AAV2VR1.6, AAV2VR1.5, AAV2VR4.1, AAV2VR4.5, AAV2VR4.2, AAV2VR4.4, AAV2VR4.3, AAV2VR4.6, AAV2EVEVRIV, AAVCBr-7_2(AAV3B), AAVCBr-7_5(AAV3B), AAVCBr-7_8(AAV3B), AAVCBr-7_4(AAV3B), CBr-B87_4(AAV5), CHt-P6(AAV5), AAVCHt-6_1(AAV5), AAVCHt-6_10(AAV5), AAVCsp8_8(AAV5), AAV6_2, Ckd-B5(AAV6), AAVCkd-B7(AAV6), AAVCkd-B8(AAV6), CKd-H3Var2(AAV6), CLv1-3(AAV9), CLv-D8(AAV9), CLv-D3(AAV9), CBr-E1(AAV9), AAVCBrE4(AAV9), 79-CLv-D5(AAV9), 91-CLv-R8(AAV9), 75Var-CLv-D1(AAV9). AAVCBr-E5(AAV9). AAVClg-F1(AAV9), AAVCsp-3(AAV9), AAVCSP11(AAV9), AAV11BC11, AAVrh8, AAVrh10, AAVrh39, AAVrh43, AAVDJ, and AAVDJ8.

2. The method of embodiment 1, wherein the AAV particle is administered via intrathecal (IT) route.

3. The method of embodiment 1, wherein the AAV particle is administered via intracerebroventricular (ICV) route.

4. The method of embodiment 1, wherein the AAV particle is administered via cisterna magna (CM) route.

5. The method of any one of embodiments 1-4, wherein the at least one brain region is selected from the group consisting of frontal cortex, occipital cortex, caudate nucleus, putamen, thalamus, hippocampus, cingulate gyrus, hypothalamus, pons, medulla, cerebellar Purkinje layer, and cerebellar granular layer.

6. The method of embodiment 5, wherein the at least one brain region is the frontal cortex.

7. The method of embodiment 5, wherein the at least one brain region is the occipital cortex.

8. The method of embodiment 5, wherein the at least one brain region is the caudate nucleus.

9. The method of embodiment 5, wherein the at least one brain region is the putamen.

10. The method of embodiment 5, wherein the at least one brain region is the thalamus.

11. The method of embodiment 5, wherein the at least one brain region is the hippocampus.

12. The method of embodiment 5, wherein the at least one brain region is the cingulate gyrus.

13. The method of embodiment 5, wherein the at least one brain region is the hypothalamus.

14. The method of embodiment 5, wherein the at least one brain region is the pons.

15. The method of embodiment 5, wherein the at least one brain region is the medulla.

16. The method of embodiment 5, wherein the at least one brain region is the cerebellar Purkinje layer.

17. The method of embodiment 5, wherein the at least one brain region is the cerebellar granular layer.

18. A method of delivering at least one payload molecule to a brain region of a subject, comprising administering at least one AAV particle to cerebrospinal fluid (CSF) of the subject, wherein the at least one AAV particle comprises a viral genome that encodes the payload molecule, and a capsid protein, whereby the at least one payload molecule is expressed in at least one brain region, and wherein at least one brain region is caudate, and whereby the capsid protein serotype is selected from the group consisting of AAV1, AAV6, AAV6mt1, and AAV6mt3.

19. The method of embodiment 18, whereby the capsid protein serotype is AAV6.

20. The method of embodiment 18, whereby the capsid protein serotype is AAV1.

21. The method of embodiment 18, whereby the capsid protein serotype is AAV6mt1.

22. The method of embodiment 18, whereby the capsid protein serotype is AAV6mt3.

23. A method of delivering at least one payload molecule to at least one brain region of a subject, comprising administering at least one AAV particle to cerebrospinal fluid (CSF) of the subject, wherein the at least one AAV particle comprises a viral genome that encodes at least one payload molecule, and a capsid protein, whereby the at least one payload molecule is expressed in at least one brain region, and wherein the brain region is selected from the group consisting of caudate, thalamus, and/or hippocampus and the capsid protein serotype is selected from the group consisting of AAV6, AAV6mt1, and AAV6mt3.

24. The method of embodiment 23, wherein the brain region is hippocampus.

25. The method of embodiment 24, wherein the capsid protein serotype is AAV6.

26. The method of embodiment 24, wherein the capsid protein serotype is AAV6mt1.

27. The method of embodiment 24, wherein the capsid protein serotype is AAV6mt3.

28. The method of embodiment 23, wherein the brain region is caudate.

29. The method of embodiment 28, wherein the capsid protein serotype is AAV6.

30. The method of embodiment 28, wherein the capsid protein serotype is AAV6mt1.

31. The method of embodiment 28, wherein the capsid protein serotype is AAV6mt3.

32. The method of embodiment 23, wherein the brain region is hippocampus.

33. The method of embodiment 32, wherein the capsid protein serotype is AAV6.

34. The method of embodiment 32, wherein the capsid protein serotype is AAV6mt1.

35. The method of embodiment 32, wherein the capsid protein serotype is AAV6mt3.

36. A method of delivering at least one payload molecule to at least one brain region of a subject, comprising administering at least one AAV particle to cerebrospinal fluid (CSF) of the subject, wherein the at least AAV particle comprises a viral genome that encodes at least one payload molecule, and a capsid protein, whereby the at least one payload molecule is expressed in the at least one brain region, and wherein the at least one brain region is thalamus and the capsid protein serotype is selected from the group consisting of AAV6, AAV6mt1, and AAV6mt3.

37. The method of embodiment 36, wherein the capsid protein serotype is AAV6.

38. The method of embodiment 36, wherein the capsid protein serotype is AAV6mt1.

39. The method of embodiment 36, wherein the capsid protein serotype is AAV6mt3.

40. A method of delivering at least one payload molecule to at least one brain region of a subject, comprising administering at least one AAV vector to cerebrospinal fluid (CSF) of the subject, wherein the at least one AAV vector comprises a viral genome that encodes at least one payload molecule, and a capsid protein, whereby the at least one payload molecule is expressed in the at least one brain region, and wherein the at least one brain region is selected from the group consisting of the caudate, thalamus and/or hypothalamus region, and the capsid protein serotype is AAV1.

41. The method of embodiment 40, wherein the at least one brain region is the caudate.

42. The method of embodiment 40, wherein the at least one brain region is the thalamus.

43. The method of embodiment 40, wherein the at least one brain region is the hypothalamus region.

44. A method of delivering at least one payload molecule to at least one brain region of a subject, comprising administering at least one AAV vector to cerebrospinal fluid (CSF) of the subject, wherein the at least one AAV vector comprises a viral genome that encodes at least one payload molecule, and a capsid protein, whereby the at least one payload molecule is expressed in at least one brain region, and wherein the at least one brain region is selected from the group consisting of the pons, medulla, and/or cerebellar cortex region and the capsid protein serotype is selected from the group consisting of AAV3B and AAV3mt4.

45. The method of embodiment 44, wherein the capsid protein serotype is AAV3B.

46. The method of embodiment 45, wherein the at least one brain region is the pons.

47. The method of embodiment 45, wherein the at least one brain region is the medulla.

48. The method of embodiment 45, wherein the at least one brain region is the cerebellar cortex region.

49. The method of embodiment 44, wherein the capsid protein serotype is AAV3mt4.

50. The method of embodiment 49, wherein the at least one brain region is the pons.

51. The method of embodiment 49, wherein the at least one brain region is the medulla.

52. The method of embodiment 49, wherein the at least one brain region is the cerebellar cortex.

53. A method of delivering at least one payload molecule to at least one brain region of a subject, comprising administering at least one AAV particle to cerebrospinal fluid (CSF) of the subject, wherein the at least one AAV particle comprises a viral genome that encodes at least one payload molecule, and a capsid protein, whereby the at least one payload molecule is expressed in the brain region, and wherein the at least one AAV particle shows at least 10-fold higher distribution in the brain region than AAV9 particle.

54. The method of embodiment 53, wherein the brain region is frontal gyrus and the capsid protein serotype is selected from the group consisting of AAV1, AAV1mt1, AAV2mt8, AAV6mt2, AAV6mt4, AAV6mt5, AAV8, AAV11, AAVrh10, AAVrh39, and AAVDJ.

55. The method of embodiment 54, wherein the capsid protein serotype is AAV1.

56. The method of embodiment 54, wherein the capsid protein serotype is AAV1mt1.

57. The method of embodiment 54, wherein the capsid protein serotype is AAV2mt8.

The method of embodiment 54, wherein the capsid protein serotype is AAV6mt2.

59. The method of embodiment 54, wherein the capsid protein serotype is AAV6mt4.

60. The method of embodiment 54, wherein the capsid protein serotype is AAV6mt5.

61. The method of embodiment 54, wherein the capsid protein serotype is AAV8.

62. The method of embodiment 54, wherein the capsid protein serotype is AAV11.

63. The method of embodiment 54, wherein the capsid protein serotype is AAVrh10.

64. The method of embodiment 54, wherein the capsid protein serotype is AAVrh39.

65. The method of embodiment 54, wherein the capsid protein serotype is AAVDJ.

66. The method of embodiment 53, wherein the brain region is occipital cortex and the capsid protein serotype is selected from the group consisting of AAV1, AAV1mt1, AAV6mt2, AAV6mt4, AAV6mt5, AAV8, AAV11, AAVrh10, AAVrh39, and AAVDJ.

67. The method of embodiment 66, wherein the capsid protein serotype is AAV1.

68. The method of embodiment 66, wherein the capsid protein serotype is AAV1mt1.

69. The method of embodiment 66, wherein the capsid protein serotype is AAV2mt8.

70. The method of embodiment 66, wherein the capsid protein serotype is AAV6mt2.

71. The method of embodiment 66, wherein the capsid protein serotype is AAV6mt4.

72. The method of embodiment 66, wherein the capsid protein serotype is AAV6mt5.

73. The method of embodiment 66, wherein the capsid protein serotype is AAV8.

74. The method of embodiment 66, wherein the capsid protein serotype is AAV11.

75. The method of embodiment 66, wherein the capsid protein serotype is AAVrh10.

76. The method of embodiment 66, wherein the capsid protein serotype is AAVrh39.

77. The method of embodiment 66, wherein the capsid protein serotype is AAVDJ.

78. The method of embodiment 53, wherein the brain region is caudate, and the capsid protein serotype is selected from the group consisting of AAV1, AAV1mt1, AAV2, AAV2mt2, AAV2mt3, AAV2mt4, AAV2mt5, AAV2mt6, AAV2mt7, AAV2mt8, AAV2mt9. AAV2mt10, AAV6, AAV6mt1, AAV6mt2, AAV6mt3, AAV6mt4, AAV6mt5, AAV9mt1. AAV9mt6, and AAVDJ.

79. The method of embodiment 78, wherein the capsid protein serotype is AAV1.

80. The method of embodiment 78, wherein the capsid protein serotype is AAV1mt1.

81. The method of embodiment 78, wherein the capsid protein serotype is AAV2mt8.

82. The method of embodiment 78, wherein the capsid protein serotype is AAV6mt2.

83. The method of embodiment 78, wherein the capsid protein serotype is AAV6mt4.

84. The method of embodiment 78, wherein the capsid protein serotype is AAV6mt5.

85. The method of embodiment 78, wherein the capsid protein serotype is AAV8.

86. The method of embodiment 78, wherein the capsid protein serotype is AAV11.

87. The method of embodiment 78, wherein the capsid protein serotype is AAVrh10.

88. The method of embodiment 78, wherein the capsid protein serotype is AAVrh39.

89. The method of embodiment 78, wherein the capsid protein serotype is AAVDJ.

90. The method of embodiment 53, wherein the brain region is putamen, and the capsid protein serotype is AAV9mt6.

91. The method of embodiment 53, wherein the brain region is hippocampus, and the capsid protein serotype is selected from the group consisting of AAV1, AAV1mt1, AAV2, AAV2mt2, AAV2mt5, AAV2mt6, AAV2mt7, AAV2mt8, AAV2mt9, AAV2mt10, AAV6, AAV6mt1, AAV6mt2, AAV6mt3, AAV6mt4, AAV6mt5, AAV9mt1, AAV9mt6, AAV11, and AAVDJ.

92. The method of embodiment 91, wherein the capsid protein serotype is AAV1.

93. The method of embodiment 91, wherein the capsid protein serotype is AAV1mt1.

94. The method of embodiment 91, wherein the capsid protein serotype is AAV2.

95. The method of embodiment 91, wherein the capsid protein serotype is AAV2mt2.

96. The method of embodiment 91, wherein the capsid protein serotype is AAV2mt5.

97. The method of embodiment 91, wherein the capsid protein serotype is AAV2mt6.

98. The method of embodiment 91, wherein the capsid protein serotype is AAV2mt7.

99. The method of embodiment 91, wherein the capsid protein serotype is AAV2mt8.

100. The method of embodiment 91, wherein the capsid protein serotype is AAV2mt9.

101. The method of embodiment 91, wherein the capsid protein serotype is AAV2mt10.

102. The method of embodiment 91, wherein the capsid protein serotype is AAV6.

103. The method of embodiment 91, wherein the capsid protein serotype is AAV6mt1.

104. The method of embodiment 91, wherein the capsid protein serotype is AAV6mt2.

105. The method of embodiment 91, wherein the capsid protein serotype is AAV6mt3.

106. The method of embodiment 91, wherein the capsid protein serotype is AAV6mt4.

107. The method of embodiment 91, wherein the capsid protein serotype is AAV6mt5.

108. The method of embodiment 91, wherein the capsid protein serotype is AAV9mt1.

109. The method of embodiment 91, wherein the capsid protein serotype is AAV9mt6.

110. The method of embodiment 91, wherein the capsid protein serotype is AAV11.

111. The method of embodiment 91, wherein the capsid protein serotype is AAVDJ.

112. The method of embodiment 53, wherein the brain region is cingulate gyms, and the capsid protein serotype is selected from the group consisting of AAV1, AAV1mt1, AAV2, AAV2mt2, AAV2mt4, AAV2mt5, AAV2mt6, AAV2mt7, AAV2mt8, AAV2mt9, AAV2mt10, AAV3B, AAV3mt1, AAV3mt2, AAV3mt3, AAV3mt4, AAV6, AAV6mt1, AAV6mt2, AAV6mt4, AAV6mt5, AAV9mt1, AAV11, AAVrh39, and AAVDJ.

113. The method of embodiment 112, wherein the capsid protein serotype is AAV1.

114. The method of embodiment 112, wherein the capsid protein serotype is AAV1mt1.

115. The method of embodiment 112, wherein the capsid protein serotype is AAV2.

116. The method of embodiment 112, wherein the capsid protein serotype is AAV2mt2.

117. The method of embodiment 112, wherein the capsid protein serotype is AAV2mt4.

118. The method of embodiment 112, wherein the capsid protein serotype is AAV2mt5.

119. The method of embodiment 112, wherein the capsid protein serotype is AAV2mt6.

120. The method of embodiment 112, wherein the capsid protein serotype is AAV2mt7.

121. The method of embodiment 112, wherein the capsid protein serotype is AAV2mt8.

122. The method of embodiment 112, wherein the capsid protein serotype is AAV2mt9.

123. The method of embodiment 112, wherein the capsid protein serotype is AAV2mt10.

124. The method of embodiment 112, wherein the capsid protein serotype is AAV3B.

125. The method of embodiment 112, wherein the capsid protein serotype is AAV3mt1.

126. The method of embodiment 112, wherein the capsid protein serotype is AAV3mt2.

127. The method of embodiment 112, wherein the capsid protein serotype is AAV3mt3.

128. The method of embodiment 112, wherein the capsid protein serotype is AAV3mt4.

129. The method of embodiment 112, wherein the capsid protein serotype is AAV6.

130. The method of embodiment 112, wherein the capsid protein serotype is AAV6mt1.

131. The method of embodiment 112, wherein the capsid protein serotype is AAV6mt2.

132. The method of embodiment 112, wherein the capsid protein serotype is AAV6mt4.

133. The method of embodiment 112, wherein the capsid protein serotype is AAV6mt5.

134. The method of embodiment 112, wherein the capsid protein serotype is AAV9mt1.

135. The method of embodiment 112, wherein the capsid protein serotype is AAV11.

136. The method of embodiment 112, wherein the capsid protein serotype is AAVrh39.

137. The method of embodiment 112, wherein the capsid protein serotype is AAVDJ.

138. The method of embodiment 53, wherein the brain region is thalamus, and the capsid protein serotype is selected from the group consisting of AAV1, AAV1mt1, AAV2, AAV2mt2, AAV2mt9, AAV4, AAV6, AAV6mt1, AAV6mt2, AAV6mt3, AAV6mt4, AAV6mt5, AAV9mt3, and AAV9mt6.

139. The method of embodiment 138, wherein the capsid protein serotype is AAV1.

140. The method of embodiment 138, wherein the capsid protein serotype is AAV1mt1.

141. The method of embodiment 138, wherein the capsid protein serotype is AAV2.

142. The method of embodiment 138, wherein the capsid protein serotype is AAV2mt2.

143. The method of embodiment 138, wherein the capsid protein serotype is AAV2mt9.

144. The method of embodiment 138, wherein the capsid protein serotype is AAV4.

145. The method of embodiment 138, wherein the capsid protein serotype is AAV6.

146. The method of embodiment 138, wherein the capsid protein serotype is AAV6mt1.

147. The method of embodiment 138, wherein the capsid protein serotype is AAV6mt2.

148. The method of embodiment 138, wherein the capsid protein serotype is AAV6mt3.

149. The method of embodiment 138, wherein the capsid protein serotype is AAV6mt4.

150. The method of embodiment 138, wherein the capsid protein serotype is AAV6mt5.

151. The method of embodiment 138, wherein the capsid protein serotype is AAV9mt3.

152. The method of embodiment 138, wherein the capsid protein serotype is AAV9mt6.

153. The method of embodiment 53, wherein the brain region is hypothalamus, and the capsid protein serotype is selected from the group consisting of AAV1, AAV1mt1, AAV2, AAV2mt2, AAV2mt3, AAV2mt4, AAV2mt5, AAV2mt6, AAV2mt7 AAV2mt8, AAV2mt9, AAV2mt10, AAV3B, AAV3mt1, AAV3mt2, AAV3mt3, AAV3mt4, AAV6mt2, AAV6mt4, AAV6mt5, AAV9mt1, AAV9mt6, AAV11, and AAVDJ.

154. The method of embodiment 153, wherein the capsid protein serotype is AAV1.

155. The method of embodiment 153, wherein the capsid protein serotype is AAV1mt1.

156. The method of embodiment 153, wherein the capsid protein serotype is AAV2.

157. The method of embodiment 153, wherein the capsid protein serotype is AAV2mt2.

158. The method of embodiment 153, wherein the capsid protein serotype is AAV2mt3.

159. The method of embodiment 153, wherein the capsid protein serotype is AAV2mt4.

160. The method of embodiment 153, wherein the capsid protein serotype is AAV2mt5.

161. The method of embodiment 153, wherein the capsid protein serotype is AAV2mt6.

162. The method of embodiment 153, wherein the capsid protein serotype is AAV2mt7.

163. The method of embodiment 153, wherein the capsid protein serotype is AAV2mt8.

164. The method of embodiment 153, wherein the capsid protein serotype is AAV2mt9.

165. The method of embodiment 153, wherein the capsid protein serotype is AAV2mt10.

166. The method of embodiment 153, wherein the capsid protein serotype is AAV3B.

167. The method of embodiment 153, wherein the capsid protein serotype is AAV3mt1.

168. The method of embodiment 153, wherein the capsid protein serotype is AAV3mt2.

169. The method of embodiment 153, wherein the capsid protein serotype is AAV3mt3.

170. The method of embodiment 153, wherein the capsid protein serotype is AAV3mt4.

171. The method of embodiment 153, wherein the capsid protein serotype is AAV6mt2.

172. The method of embodiment 153, wherein the capsid protein serotype is AAV6mt4.

173. The method of embodiment 153, wherein the capsid protein serotype is AAV6mt5.

174. The method of embodiment 153, wherein the capsid protein serotype is AAV9mt1.

175. The method of embodiment 153, wherein the capsid protein serotype is AAV9mt6.

176. The method of embodiment 153, wherein the capsid protein serotype is AAV11.

177. The method of embodiment 153, wherein the capsid protein serotype is AAVDJ.

178. The method of embodiment 53, wherein the brain region is pons, and the capsid protein serotype is selected from the group consisting of AAV1, AAV1mt1, AAV2, AAV2mt2, AAV2mt4, AAV2mt5, AAV2mt6, AAV2mt7, AAV2mt8, AAV2mt10. AAV6mt4, AAV6mt5, AAV9mt1, AAV9mt6, AAV11, and AAVDJ.

179. The method of embodiment 178, wherein the capsid protein serotype is AAV1.

180. The method of embodiment 178, wherein the capsid protein serotype is AAV1mt1.

181. The method of embodiment 178, wherein the capsid protein serotype is AAV2.

182. The method of embodiment 178, wherein the capsid protein serotype is AAV2mt2.

183. The method of embodiment 178, wherein the capsid protein serotype is AAV2mt4.

184. The method of embodiment 178, wherein the capsid protein serotype is AAV2mt5.

185. The method of embodiment 178, wherein the capsid protein serotype is AAV2mt6.

186. The method of embodiment 178, wherein the capsid protein serotype is AAV2mt7.

187. The method of embodiment 178, wherein the capsid protein serotype is AAV2mt8.

188. The method of embodiment 178, wherein the capsid protein serotype is AAV2mt10.

189. The method of embodiment 178, wherein the capsid protein serotype is AAV6mt4.

190. The method of embodiment 178, wherein the capsid protein serotype is AAV6mt5.

191. The method of embodiment 178, wherein the capsid protein serotype is AAV9mt1.

192. The method of embodiment 178, wherein the capsid protein serotype is AAV9mt6.

193. The method of embodiment 178, wherein the capsid protein serotype is AAV11.

194. The method of embodiment 178, wherein the capsid protein serotype is AAVDJ.

195. The method of embodiment 53, wherein the brain region is medulla, and the capsid protein serotype is selected from the group consisting of AAV1, AAV1mt1, AAV2, AAV2mt2, AAV2mt3, AAV2mt4, AAV2mt5, AAV2mt6, AAV2mt7, AAV2mt8, AAV2mt9, AAV2mt10, AAV3B, AAV3mt1, AAV3mt2, AAV3mt3, AAV3mt4, AAV6mt2, AAV6mt4, AAV6mt5, AAV9mt1, AAV11, AAVrh39, and AAVDJ.

196. The method of embodiment 195, wherein the capsid protein serotype is AAV1.

197. The method of embodiment 195, wherein the capsid protein serotype is AAV1mt1.

198. The method of embodiment 195, wherein the capsid protein serotype is AAV2.

199. The method of embodiment 195, wherein the capsid protein serotype is AAV2mt2.

200. The method of embodiment 195, wherein the capsid protein serotype is AAV2mt3.

201. The method of embodiment 195, wherein the capsid protein serotype is AAV2mt4.

202. The method of embodiment 195, wherein the capsid protein serotype is AAV2mt5.

203. The method of embodiment 195, wherein the capsid protein serotype is AAV2mt6.

204. The method of embodiment 195, wherein the capsid protein serotype is AAV2mt7.

205. The method of embodiment 195, wherein the capsid protein serotype is AAV2mt8.

206. The method of embodiment 195, wherein the capsid protein serotype is AAV2mt9.

207. The method of embodiment 195, wherein the capsid protein serotype is AAV2mt10.

208. The method of embodiment 195, wherein the capsid protein serotype is AAV3B.

209. The method of embodiment 195, wherein the capsid protein serotype is AAV3mt1.

210. The method of embodiment 195, wherein the capsid protein serotype is AAV3mt2.

211. The method of embodiment 195, wherein the capsid protein serotype is AAV3mt3.

210. The method of embodiment 195, wherein the capsid protein serotype is AAV3mt4.

213. The method of embodiment 195, wherein the capsid protein serotype is AAV6mt2.

214. The method of embodiment 195, wherein the capsid protein serotype is AAV6mt4.

215. The method of embodiment 195, wherein the capsid protein serotype is AAV6mt5.

216. The method of embodiment 195, wherein the capsid protein serotype is AAV9mt1.

217. The method of embodiment 195, wherein the capsid protein serotype is AAV11.

218. The method of embodiment 195, wherein the capsid protein serotype is AAVrh39.

219. The method of embodiment 195, wherein the capsid protein serotype is AAVDJ.

220. The method of embodiment 53, wherein the brain region is cerebellar Purkinje layer, and the capsid protein serotype is selected from the group consisting of AAV1, AAV1mt1, AAV2, AAV2mt2, AAV2mt5, AAV2mt6, AAV2mt7, AAV2mt8, AAV2mt9, AAV2mt10, AAV6mt2, AAV6mt4, AAV6mt5, AAV8, AAV9mt1, AAV11, AAVrh10, AAVrh39, and AAVDJ.

221. The method of embodiment 220, wherein the capsid protein serotype is AAV1.

222. The method of embodiment 220, wherein the capsid protein serotype is AAV1mt1.

223. The method of embodiment 220, wherein the capsid protein serotype is AAV2.

224. The method of embodiment 220, wherein the capsid protein serotype is AAV2mt2.

225. The method of embodiment 220, wherein the capsid protein serotype is AAV2mt5.

226. The method of embodiment 220, wherein the capsid protein serotype is AAV2mt6.

227. The method of embodiment 220, wherein the capsid protein serotype is AAV2mt7.

228. The method of embodiment 220, wherein the capsid protein serotype is AAV2mt8.

229. The method of embodiment 220, wherein the capsid protein serotype is AAV2mt9.

230. The method of embodiment 220, wherein the capsid protein serotype is AAV2mt10.

231. The method of embodiment 220, wherein the capsid protein serotype is AAV6mt2.

232. The method of embodiment 220, wherein the capsid protein serotype is AAV6mt4.

233. The method of embodiment 220, wherein the capsid protein serotype is AAV6mt5.

234. The method of embodiment 220, wherein the capsid protein serotype is AAV8.

235. The method of embodiment 220, wherein the capsid protein serotype is AAV9mt1.

236. The method of embodiment 220, wherein the capsid protein serotype is AAV11.

237. The method of embodiment 220, wherein the capsid protein serotype is AAVrh10.

238. The method of embodiment 220, wherein the capsid protein serotype is AAVrh39.

239. The method of embodiment 220, wherein the capsid protein serotype is AAVDJ.

240. The method of embodiment 53 wherein the brain region is cerebellar granular layer, and the capsid protein serotype is selected from the group consisting of AAV1, AAV1mt1, AAV2, AAV2mt2, AAV2mt5, AAV2mt6, AAV2mt7, AAV2mt8, AAV2mt9, AAV2mt10, AAV6, AAV6mt1, AAV6mt2, AAV6mt3, AAV6mt4, AAV6mt1, AAV8. AAV9mt1, AAV9mt6, AAV11, AAVrh10, AAVrh39, and AAVDJ.

241. The method of embodiment 240, wherein the capsid protein serotype is AAV1.

242. The method of embodiment 240, wherein the capsid protein serotype is AAV1mt1.

243. The method of embodiment 240, wherein the capsid protein serotype is AAV2.

244. The method of embodiment 240, wherein the capsid protein serotype is AAV2mt2.

245. The method of embodiment 240, wherein the capsid protein serotype is AAV2mt5.

246. The method of embodiment 240, wherein the capsid protein serotype is AAV2mt6.

247. The method of embodiment 240, wherein the capsid protein serotype is AAV2mt7.

248. The method of embodiment 240, wherein the capsid protein serotype is AAV2mt8.

249. The method of embodiment 240, wherein the capsid protein serotype is AAV2mt9.

250. The method of embodiment 240, wherein the capsid protein serotype is AAV2mt10.

251. The method of embodiment 240, wherein the capsid protein serotype is AAV6.

252. The method of embodiment 240, wherein the capsid protein serotype is AAV6mt1.

253. The method of embodiment 240, wherein the capsid protein serotype is AAV6mt3.

254. The method of embodiment 240, wherein the capsid protein serotype is AAV6mt4.

255. The method of embodiment 240, wherein the capsid protein serotype is AAV6mt5.

256. The method of embodiment 240, wherein the capsid protein serotype is AAV8.

257. The method of embodiment 240, wherein the capsid protein serotype is AAV9mt1.

258. The method of embodiment 240, wherein the capsid protein serotype is AAV9mt6.

259. The method of embodiment 240, wherein the capsid protein serotype is AAV11.

260. The method of embodiment 240, wherein the capsid protein serotype is AAVrh10.

261. The method of embodiment 240, wherein the capsid protein serotype is AAVrth39.

262. The method of embodiment 240, wherein the capsid protein serotype is AAVDJ.

263. The method of any one of embodiments 53-262, whereby the distribution in the brain is measured by DNA bar coding.

264. The method of delivering at least one payload molecule to at least one brain region of a subject, comprising administering at least one AAV particle to cembrospinal fluid (CSF) of the subject, wherein the at least one AAV particle comprises a viral genome that encodes the payload molecule, and a capsid protein, whereby the at least one payload molecule is expressed in the brain region, and wherein the at least one AAV particle shows at least 10-fold higher expression in the brain region than AAV9 particle.

265. The method of embodiment 264, wherein the brain region is frontal gyrus and the capsid protein is selected from the group consisting of AAV1, AAV1mt1, AAV2mt8. AAV6mt2, AAV6mt4, AAV6mt5, AAV8, AAV11, AAVrh10, AAVrh39, and AAVDJ.

266. The method of embodiment 265, wherein the capsid protein serotype is AAV1.

267. The method of embodiment 265, wherein the capsid protein serotype is AAV1mt1.

268. The method of embodiment 265, wherein the capsid protein serotype is AAV2mt8.

269. The method of embodiment 265, wherein the capsid protein serotype is AAV6mt2.

270. The method of embodiment 265, wherein the capsid protein serotype is AAV6mt4.

271. The method of embodiment 265, wherein the capsid protein serotype is AAV6mt5.

272. The method of embodiment 265, wherein the capsid protein serotype is AAV8.

273. The method of embodiment 265, wherein the capsid protein serotype is AAV11.

274. The method of embodiment 265, wherein the capsid protein serotype is AAVrh10.

275. The method of embodiment 265, wherein the capsid protein serotype is AAVrh39.

276. The method of embodiment 265, wherein the capsid protein serotype is AAVDJ.

277. The method of embodiment 264, wherein the brain region is caudate, and the capsid protein serotype is selected from the group consisting of AAV1, AAV1mt1, AAV2, AAV2mt2, AAV2mt10, AAV6, and AAV6mt1.

278. The method of embodiment 277, wherein the capsid protein serotype is AAV1.

279. The method of embodiment 277, wherein the capsid protein serotype is AAV1mt1.

280. The method of embodiment 277, wherein the capsid protein serotype is AAV2.

281. The method of embodiment 277, wherein the capsid protein serotype is AAV2mt2.

282. The method of embodiment 277, wherein the capsid protein serotype is AAV2mt10.

283. The method of embodiment 277, wherein the capsid protein serotype is AAV6.

284. The method of embodiment 277, wherein the capsid protein serotype is AAV6mt1.

285. The method of embodiment 264, wherein the brain region is hippocampus, and the capsid protein serotype is selected from the group consisting of AAV6, AAV6mt1, and AAV6mt3.

286. The method of embodiment 285, wherein the capsid protein serotype is AAV6.

287. The method of embodiment 285, wherein the capsid protein serotype is AAV6mt1.

288. The method of embodiment 285, wherein the capsid protein serotype is AAV6mt3.

289. The method of embodiment 264, wherein the brain region is thalamus, and the capsid protein serotype is selected from the group consisting of AAV1, AAV2, AAV2mt2, AAV6, AAV6mt1, AAV6mt3, AAV6mt5, AAV9mt6.

290. The method of embodiment 289, wherein the capsid protein serotype is AAV1.

291. The method of embodiment 289, wherein the capsid protein serotype is AAV2.

292. The method of embodiment 289, wherein the capsid protein serotype is AAV2mt2.

293. The method of embodiment 289, wherein the capsid protein serotype is AAV6.

294. The method of embodiment 289, wherein the capsid protein serotype is AAV6mt1.

295. The method of embodiment 289, wherein the capsid protein serotype is AAV6mt3.

296. The method of embodiment 289, wherein the capsid protein serotype is AAV6mt5.

297. The method of embodiment 289, wherein the capsid protein serotype is AAV9mt6.

298. The method of embodiment 264, wherein the brain region is hypothalamus, and the capsid protein serotype is selected from the group consisting of AAV2, AAV2mt2, AAV2mt5, AAV2mt9, AAV9mt6, and AAVDJ.

299. The method of embodiment 298, wherein the capsid protein serotype is AAV2.

300. The method of embodiment 298, wherein the capsid protein serotype is AAV2mt2.

301. The method of embodiment 298, wherein the capsid protein serotype is AAV2mt5.

302. The method of embodiment 298, wherein the capsid protein serotype is AAV2mt9.

303. The method of embodiment 298, wherein the capsid protein serotype is AAV9mt6.

304. The method of embodiment 298, wherein the capsid protein serotype is AAVDJ.

305. The method of embodiment 264, wherein the brain region is pons, and the capsid protein serotype is selected from the group consisting of AAV3B and AAV3mt4.

306. The method of embodiment 305, wherein the capsid protein serotype is AAV3B.

307. The method of embodiment 305, wherein the capsid protein serotype is AAV3mt4.

308. The method of embodiment 264, wherein the brain region is medulla, and the capsid protein serotype is selected from the group consisting of AAV3B and AAV3mt4.

309. The method of embodiment 308, wherein the capsid protein serotype is AAV3B.

310. The method of embodiment 308, wherein the capsid protein serotype is AAV3mt4.

311. The method of embodiment 264, wherein the brain region is cerebellar Purkinje layer, and the capsid protein is selected from the group consisting of AAV3B and AAV3mt4.

312. The method of embodiment 311, wherein the capsid protein serotype is AAV3B.

313. The method of embodiment 311, wherein the capsid protein serotype is AAV3mt4.

314. The method of embodiment 264, wherein the brain region is cerebellar Granular layer, and the capsid protein is selected from the group consisting of AAV6 and AAV6mt1.

315. The method of embodiment 314, wherein the capsid protein serotype is AAV6.

316. The method of embodiment 314, wherein the capsid protein serotype is AAV6mt1.

317. The method of any one of embodiments 264-316, whereby expression in the brain region is measured by RNA bar coding.

318. A method of delivering at least one payload molecule to at least one brain region of a subject, comprising administering at least one AAV particle to cerebrospinal fluid (CSF) of the subject, wherein the at least one AAV particle comprises a viral genome that encodes at least one payload molecule, and a capsid protein, whereby the at least one payload molecule is expressed in the at least one brain region, and wherein the at least one AAV particle shows at least 20-fold higher distribution in the brain region than AAV9 particle.

319. The method of embodiment 318, wherein the brain region is frontal gyrus and the capsid protein serotype is selected from the group consisting of AAV1, AAV1mt1, AAV6mt2, AAV6mt5, AAV11, and AAVDJ.

320. The method of embodiment 319, wherein the capsid protein serotype is AAV1.

321. The method of embodiment 319, wherein the capsid protein serotype is AAV1mt1.

322. The method of embodiment 319, wherein the capsid protein serotype is AAV6mt2.

323. The method of embodiment 319, wherein the capsid protein serotype is AAV6mt5.

324. The method of embodiment 319, wherein the capsid protein serotype is AAV11.

325. The method of embodiment 319, wherein the capsid protein serotype is AAVDJ.

326. The method of embodiment 318, wherein the brain region is occipital cortex and the capsid protein is selected from the group consisting of AAV1, AAV1mt1, AAV6mt2. AAV6mt5, and AAV11.

327. The method of embodiment 326, wherein the capsid protein serotype is AAV1.

328. The method of embodiment 326, wherein the capsid protein serotype is AAV1mt1.

329. The method of embodiment 326, wherein the capsid protein serotype is AAV6mt2.

330. The method of embodiment 326, wherein the capsid protein serotype is AAV6mt5.

331. The method of embodiment 326, wherein the capsid protein serotype is AAV11.

332. The method of embodiment 319, wherein the capsid protein serotype is selected from the group consisting of AAV6mt5, AAV11, AAVrh10, and AAVrh39.

333. The method of embodiment 332, wherein the capsid protein serotype is AAV6mt5.

334. The method of embodiment 332, wherein the capsid protein serotype is AAV11.

335. The method of embodiment 332, wherein the capsid protein serotype is AAVrh10.

334. The method of embodiment 332, wherein the capsid protein serotype is AAVrh39.

337. The method of embodiment 318, wherein the brain region is caudate, and the capsid protein serotype is selected from the group consisting of AAV1, AAV1mt1, AAV2, AAV2mt2, AAV2mt3, AAV2mt4, AAV2mt5, AAV2mt7, AAV2mt8, AAV2mt9, AAV2mt10, AAV6, AAV6mt1, AAV6mt2, AAV6mt3, AAV6mt4, AAV6mt5, AAV9mt1. AAV9mt6, and AAVDJ.

338. The method of embodiment 337, wherein the capsid protein serotype is AAV1.

339. The method of embodiment 337, wherein the capsid protein serotype is AAV1mt1.

340. The method of embodiment 337, wherein the capsid protein serotype is AAV2.

341. The method of embodiment 337, wherein the capsid protein serotype is AAV2mt2.

342. The method of embodiment 337, wherein the capsid protein serotype is AAV2mt3.

343. The method of embodiment 337, wherein the capsid protein serotype is AAV2mt4.

344. The method of embodiment 337, wherein the capsid protein serotype is AAV2mt5.

345. The method of embodiment 337, wherein the capsid protein serotype is AAV2mt7.

346. The method of embodiment 337, wherein the capsid protein serotype is AAV2mt8.

347. The method of embodiment 337, wherein the capsid protein serotype is AAV2mt9.

348. The method of embodiment 337, wherein the capsid protein serotype is AAV2mt10.

349. The method of embodiment 337, wherein the capsid protein serotype is AAV6.

350. The method of embodiment 337, wherein the capsid protein serotype is AAV6mt1.

351. The method of embodiment 337, wherein the capsid protein serotype is AAV6mt2.

352. The method of embodiment 337, wherein the capsid protein serotype is AAV6mt3.

353. The method of embodiment 337, wherein the capsid protein serotype is AAV6mt4.

354. The method of embodiment 337, wherein the capsid protein serotype is AAV6mt5.

355. The method of embodiment 337, wherein the capsid protein serotype is AAV9mt1.

356. The method of embodiment 337, wherein the capsid protein serotype is AAV9mt6.

357. The method of embodiment 337, wherein the capsid protein serotype is AAVDJ.

358. The method of embodiment 318, wherein the brain region is putamen, and the capsid protein is AAV9mt6.

359. The method of embodiment 318, wherein the brain region is hippocampus, and the capsid protein serotype is selected from the group consisting of AAV1mt1, AAV2, AAV2m5, AAV2mt6, AAV2mt7, AAV2mt8, AAV2mt9, AAV6, AAV6mt1, AAV6mt2.

AAV6mt3, AAV6mt4, AAV6mt5, AAV9mt1, AAV9mt6, AAV11, and AAVDJ.

360. The method of embodiment 359, wherein the capsid protein serotype is AAV1mt1.

361. The method of embodiment 359, wherein the capsid protein serotype is AAV2.

362. The method of embodiment 359, wherein the capsid protein serotype is AAV2mt5.

363. The method of embodiment 359, wherein the capsid protein serotype is AAV2mt6.

364. The method of embodiment 359, wherein the capsid protein serotype is AAV2mt7.

365. The method of embodiment 359, wherein the capsid protein serotype is AAV2mt8.

366. The method of embodiment 359, wherein the capsid protein serotype is AAV2mt9.

367. The method of embodiment 359, wherein the capsid protein serotype is AAV6.

368. The method of embodiment 359, wherein the capsid protein serotype is AAV6mt1.

369. The method of embodiment 359, wherein the capsid protein serotype is AAV6mt2.

370. The method of embodiment 359, wherein the capsid protein serotype is AAV6mt3.

371. The method of embodiment 359, wherein the capsid protein serotype is AAV6mt4.

372. The method of embodiment 359, wherein the capsid protein serotype is AAV6mt5.

373. The method of embodiment 359, wherein the capsid protein serotype is AAV9mt1.

374. The method of embodiment 359, wherein the capsid protein serotype is AAV9mt6.

375. The method of embodiment 359, wherein the capsid protein serotype is AAV11.

376. The method of embodiment 359, wherein the capsid protein serotype is AAVDJ.

377. The method of embodiment 318, wherein the brain region is cingulate gyrus, and the capsid protein serotype is selected from the group consisting of AAV1mt1, AAV2mt2, AAV2mt4, AAV2mt5, AAV2mt6, AAV2mt7, AAV2mt8, AAV2mt10, AAV3B, AAV3mt1, AAV3mt2, AAV3mt3, AAV3mt4, AAV6, AAV6mt1, AAV6mt2, AAV6mt4, AAV6mt5, AAV9mt1, AAV11, and AAVDJ.

378. The method of embodiment 377, wherein the capsid protein serotype is AAV1mt1.

379. The method of embodiment 377, wherein the capsid protein serotype is AAV2mt2.

380. The method of embodiment 377, wherein the capsid protein serotype is AAV2mt4.

381. The method of embodiment 377, wherein the capsid protein serotype is AA2mt5.

382. The method of embodiment 377, wherein the capsid protein serotype is AAV2mt6.

383. The method of embodiment 377, wherein the capsid protein serotype is AAV2mt7.

384. The method of embodiment 377, wherein the capsid protein serotype is AAV2mt8.

385. The method of embodiment 377, wherein the capsid protein serotype is AAV2mt10.

386. The method of embodiment 377, wherein the capsid protein serotype is AAV3B.

387. The method of embodiment 377, wherein the capsid protein serotype is AAV3mt1.

388. The method of embodiment 377, wherein the capsid protein serotype is AAV3mt2.

389. The method of embodiment 377, wherein the capsid protein serotype is AAV3mt3.

390. The method of embodiment 377, wherein the capsid protein serotype is AAV3mt4.

391. The method of embodiment 377, wherein the capsid protein serotype is AAV6.

392. The method of embodiment 377, wherein the capsid protein serotype is AAV6mt1.

393. The method of embodiment 377, wherein the capsid protein serotype is AAV6mt2.

394. The method of embodiment 377, wherein the capsid protein serotype is AAV6mt4.

395. The method of embodiment 377, wherein the capsid protein serotype is AAV6mt5.

396. The method of embodiment 377, wherein the capsid protein serotype is AAV9mt1.

397. The method of embodiment 377, wherein the capsid protein serotype is AAV11.

398. The method of embodiment 377, wherein the capsid protein serotype is AAVDJ.

399. The method of embodiment 318, wherein the brain region is thalamus, and the capsid protein serotype is selected from the group consisting of AAV1, AAV1mt1, AAV2, AAV2mt2, AAV2mt9, AAV4, AAV6, AAV6mt1, AAV6mt2, AAV6mt3, AAV6mt4, AAV6mt5, AAV9mt3, and AAV9mt6.

400. The method of embodiment 399, wherein the capsid protein serotype is AAV1.

401. The method of embodiment 399, wherein the capsid protein serotype is AAV1mt1.

402. The method of embodiment 399, wherein the capsid protein serotype is AAV2.

403. The method of embodiment 399, wherein the capsid protein serotype is AAV2mt2.

404. The method of embodiment 399, wherein the capsid protein serotype is AAV2mt9.

405. The method of embodiment 399, wherein the capsid protein serotype is AAV4.

406. The method of embodiment 399, wherein the capsid protein serotype is AAV6.

407. The method of embodiment 399, wherein the capsid protein serotype is AAV6mt1.

408. The method of embodiment 399, wherein the capsid protein serotype is AAV6mt3.

409. The method of embodiment 399, wherein the capsid protein serotype is AAV6mt4.

410. The method of embodiment 399, wherein the capsid protein serotype is AAV6mt5.

411. The method of embodiment 399, wherein the capsid protein serotype is AAV9mt3.

412. The method of embodiment 399, wherein the capsid protein serotype is AAV9mt6.

413. The method of embodiment 318, wherein the brain region is hypothalamus, and the capsid protein serotype is selected from the group consisting of AAV2, AAV2mt2, AAV2mt4, AAV2mt5, AAV2mt6, AAV2mt7, AAV2mt8, AAV2mt9, AAV2mt10, AAV3B, AAV3mt1, AAV3mt2, AAV3mt3, AAV3mt4, AAV6mt5, AAV9mt1, AAV9mt6, AAV11, and AAVDJ.

414. The method of embodiment 413, wherein the capsid protein serotype is AAV2.

415. The method of embodiment 413, wherein the capsid protein serotype is AAV2mt2.

416. The method of embodiment 413, wherein the capsid protein serotype is AAV2mt4.

417. The method of embodiment 413, wherein the capsid protein serotype is AAV2mt5.

418. The method of embodiment 413, wherein the capsid protein serotype is AAV2mt6.

419. The method of embodiment 413, wherein the capsid protein serotype is AAV2mt7.

420. The method of embodiment 413, wherein the capsid protein serotype is AAV2mt8.

421. The method of embodiment 413, wherein the capsid protein serotype is AAV2mt9.

422. The method of embodiment 413, wherein the capsid protein serotype is AAV2mt10.

423. The method of embodiment 413, wherein the capsid protein serotype is AAV3B.

424. The method of embodiment 413, wherein the capsid protein serotype is AAV3mt1.

425. The method of embodiment 413, wherein the capsid protein serotype is AAV3mt2.

426. The method of embodiment 413, wherein the capsid protein serotype is AAV3mt3.

427. The method of embodiment 413, wherein the capsid protein serotype is AAV3mt4.

428. The method of embodiment 413, wherein the capsid protein serotype is AAV6mt5.

429. The method of embodiment 413, wherein the capsid protein serotype is AAV9mt1.

430. The method of embodiment 413, wherein the capsid protein serotype is AAV9mt6.

431. The method of embodiment 413, wherein the capsid protein serotype is AAV11.

432. The method of embodiment 413, wherein the capsid protein serotype is AAVDJ.

433. The method of embodiment 318, wherein the brain region is pons, and the capsid protein serotype is selected from the group consisting of AAV1mt1, AAV2, AAV2mt2, AAV2mt4, AAV2mt5, AAV2mt6, AAV2mt7, AAV2mt8, AAV2mt10, AAV6mt5, AAV11, and AAVDJ.

434. The method of embodiment 433, wherein the capsid protein serotype is AAV1mt1.

435. The method of embodiment 433, wherein the capsid protein serotype is AAV2.

436. The method of embodiment 433, wherein the capsid protein serotype is AAV2mt2.

437. The method of embodiment 433, wherein the capsid protein serotype is AAV2mt4.

438. The method of embodiment 433, wherein the capsid protein serotype is AAV2mt5.

439. The method of embodiment 433, wherein the capsid protein serotype is AAV2mt6.

440. The method of embodiment 433, wherein the capsid protein serotype is AAV2mt7.

441. The method of embodiment 433, wherein the capsid protein serotype is AAV2mt8.

442. The method of embodiment 433, wherein the capsid protein serotype is AAV2mt10.

443. The method of embodiment 433, wherein the capsid protein serotype is AAV6mt5.

444. The method of embodiment 433, wherein the capsid protein serotype is AAV11.

445. The method of embodiment 433, wherein the capsid protein serotype is AAVDJ.

446. The method of embodiment 318, wherein the brain region is medulla, and the capsid protein is selected from the group consisting of AAV1, AAV1mt1, AAV2, AAV2mt2, AAV2mt3, AAV2mt4, AAV2mt5, AAV2mt6, AAV2mt7, AAV2mt8, AAV2mt9, AAV2mt10, AAV3B, AAV3mt1, AAV3mt2, AAV3mt3, AAV3mt4, AAV6mt5, AAV9mt1, AAV11, AAVrh39, and AAVDJ.

447. The method of embodiment 318, wherein the brain region is cerebellar Purkinje layer, and the capsid protein is selected from the group consisting of AAV1, AAV1mt1, AAV2mt5, AAV2mt7, AAV2mt8, AAV6mt2, AAV6mt5, AAV11, and AAVDJ.

448. The method of embodiment 447, wherein the capsid protein serotype is AAV1.

449. The method of embodiment 447, wherein the capsid protein serotype is AAV1mt1.

450. The method of embodiment 447, wherein the capsid protein serotype is AAV2mt5.

451. The method of embodiment 447, wherein the capsid protein serotype is AAV2mt7.

452. The method of embodiment 447, wherein the capsid protein serotype is AAV2mt8.

453. The method of embodiment 447, wherein the capsid protein serotype is AAV6mt2.

454. The method of embodiment 447, wherein the capsid protein serotype is AAV6mt5.

455. The method of embodiment 447, wherein the capsid protein serotype is AAV11.

456. The method of embodiment 447, wherein the capsid protein serotype is AAVDJ.

457. The method of embodiment 318, wherein the brain region is cerebellar Granular layer, and the capsid protein is selected from the group consisting of AAV1, AAV1mt1, AAV2, AAV2mt2, AAV2mt5, AAV2mt7, AAV2mt8, AAV6, AAV6mt1, AAV6mt2, AAV6mt3, AAV6mt4, AAV6mt5, AAV9mt6, AAV11, and AAVDJ.

458. The method of embodiment 457, wherein the capsid protein serotype is AAV1.

459. The method of embodiment 457, wherein the capsid protein serotype is AAV1mt1.

460. The method of embodiment 457, wherein the capsid protein serotype is AAV2.

461. The method of embodiment 457, wherein the capsid protein serotype is AAV2mt2.

462. The method of embodiment 457, wherein the capsid protein serotype is AAV2mt5.

463. The method of embodiment 457, wherein the capsid protein serotype is AAV2mt7.

465. The method of embodiment 457, wherein the capsid protein serotype is AAV2mt8.

466. The method of embodiment 457, wherein the capsid protein serotype is AAV6.

467. The method of embodiment 457, wherein the capsid protein serotype is AAV6mt1.

468. The method of embodiment 457, wherein the capsid protein serotype is AAV6mt2.

469. The method of embodiment 457, wherein the capsid protein serotype is AAV6mt3.

470. The method of embodiment 457, wherein the capsid protein serotype is AAV6mt4.

471. The method of embodiment 457, wherein the capsid protein serotype is AAV6mt5.

472. The method of embodiment 457, wherein the capsid protein serotype is AAV9mt6.

473. The method of embodiment 457, wherein the capsid protein serotype is AAV11.

474. The method of embodiment 457, wherein the capsid protein serotype is AAVDJ.

475. The method of any one of embodiments 318-474, whereby the distribution in the brain is measured by DNA bar coding.

476. A method of delivering at least one payload molecule to at least one brain region of a subject, comprising administering at least one AAV particle to cerebrospinal fluid (CSF) of the subject, wherein the at least one AAV particle comprises a viral genome that encodes the at least one payload molecule, and a capsid protein, whereby the at least one payload molecule is expressed in the at least one brain region, and wherein the at least one AAV particle shows at least 50-fold higher distribution in the brain region than AAV9 particle.

477. The method of embodiment 476, wherein the brain region is caudate, and the capsid protein serotype is AAV2.

478. The method of embodiment 476, wherein the brain region is hypothalamus, and the capsid protein serotype is selected from the group consisting of AAV2, AAV2mt2, AAV2mt5, AAV9mt6, AAV11, and AAVDJ.

479. The method of embodiment 478, wherein the capsid protein serotype is AAV2.

480. The method of embodiment 478, wherein the capsid protein serotype is AAV2mt5.

481. The method of embodiment 478, wherein the capsid protein serotype is AAV2mt5.

482. The method of embodiment 478, wherein the capsid protein serotype is AAV9mt6.

483. The method of embodiment 478, wherein the capsid protein serotype is AAV11.

484. The method of embodiment 478, wherein the capsid protein serotype is AAVDJ.

485. The method of embodiment 476, wherein the brain region is medulla, and the capsid protein serotype is selected from the group consisting of AAV2, AAV2mt2, AAV2mt6, AAV2mt7, AAV2mt8, AAV2mt9, AAV2mt10, AAV11, and AAVDJ.

486. The method of embodiment 485, wherein the capsid protein serotype is AAV2.

487. The method of embodiment 485, wherein the capsid protein serotype AAV2mt2.

488. The method of embodiment 485, wherein the capsid protein serotype AAV2mt6.

489. The method of embodiment 485, wherein the capsid protein serotype AAV2mt7.

490. The method of embodiment 485, wherein the capsid protein serotype AAV2mt8.

491. The method of embodiment 485, wherein the capsid protein serotype AAV2mt9.

492. The method of embodiment 485, wherein the capsid protein serotype AAV2mt10.

493. The method of embodiment 485, wherein the capsid protein serotype AAV11.

494. The method of embodiment 485, wherein the capsid protein serotype AAVDJ.

495. The method of embodiment 476, wherein the brain region is cerebellar Purkinje layer, and the capsid protein serotype is AAV11.

496. The method of any one of embodiments 476-495, whereby the distribution in the brain is measured by DNA bar coding.

497. A method of delivering at least one payload molecule to at least one brain region of a subject, comprising administering at least one AAV particle to cerebrospinal fluid (CSF) of the subject, wherein the at least one AAV particle comprises a viral genome that encodes the at least one payload molecule, and a capsid protein, whereby the at least one payload molecule is expressed in the brain region, and wherein the at least one AAV particle shows at least 20-fold higher expression in the at least one brain region than AAV9 particle.

498. The method of embodiment 497, wherein the brain region is caudate, and the capsid protein serotype is AAV6.

499. A method of embodiment 497, wherein the brain region is thalamus, and the capsid protein serotype is selected from the group consisting of AAV6, AAV6mt1, and AAV6mt3.

500. The method of embodiment 499, wherein the capsid protein serotype is AAV6.

501. The method of embodiment 499, wherein the capsid protein serotype is AAV6mt1.

502. The method of embodiment 499, wherein the capsid protein serotype is AAV6mt3.

503. The method of any one of embodiments 497-502, whereby expression in the brain region is measured by RNA bar coding.

504. The method of any one of embodiments 1-503, wherein one of the at least one payload molecules is a polynucleotide.

505. The method of embodiment 504, wherein the polynucleotide is an siRNA duplex.

506. The method of embodiment 505, wherein the siRNA duplex, when expressed, inhibits or suppresses the expression of a gene of interest in a cell.

507. The method of embodiment 506, wherein the gene of interest is selected from the group consisting of superoxide dismutase 1 (SOD1), chromosome 9 open reading frame 72 (C9ORF72), TAR DNA binding protein (TARDBP), ataxin 3 (ATXN3), huntingtin (HTT), amyloid precursor protein (APP), apolipoprotein E (APOE), microtubule-associated protein tau (MAPT), alpha synuclein (SNCA), voltage-gated sodium channel alpha subunit 9 (SCN9A), and voltage-gated sodium channel alpha subunit 10 (SCN10A).

508. The method of embodiment 507, wherein the gene of interest is SOD1.

509. The method of embodiment 507, wherein the gene of interest is C9ORF72.

510. The method of embodiment 507, wherein the gene of interest is TARDBP.

511. The method of embodiment 507, wherein the gene of interest is ATXN3.

512. The method of embodiment 507, wherein the gene of interest is HTT.

513. The method of embodiment 507, wherein the gene of interest is APP.

514. The method of embodiment 507, wherein the gene of interest is APOE.

515. The method of embodiment 507, wherein the gene of interest is MAPT.

515. The method of embodiment 507, wherein the gene of interest is SNCA.

516. The method of embodiment 507, wherein the gene of interest is SCN9A.

517. The method of embodiment 507, wherein the gene of interest is SCN10A.

518. The method of any one of embodiments 1-503, wherein one of the at least one payload molecules is a polypeptide.

519. The method of embodiment 518, wherein the polypeptide is selected from the group consisting of an antibody, Aromatic L-Amino Acid Decarboxylase (AADC), APOE2, Frataxin (FXN), survival motor neuron (SMN) protein, glucocerebrosidase (GCase), N-sulfoglucosamine sulfohydrolase, N-acetyl-alpha-glucosaminidase, iduronate 2-sulfatase, alpha-L-iduronidase, palmitoyl-protein thioesterase 1, tripeptidyl peptidase 1, battenin, CLN5, CLN6 (linclin), MFSD8, CLN8, aspartoacylase (ASPA), progranulin (GRN), MeCP2, beta-galactosidase (GLB1), gigaxonin (GAN), ATPase Sarcoplasmic/Endoplasmic Reticulum Ca2+ Transporting 2 (ATP2A2), and S100 Calcium Binding Protein A1 (S100A1).

520. The method of embodiment 519, wherein the polypeptide is AADC.

521. The method of embodiment 519, wherein the polypeptide is APOE2.

522. The method of embodiment 519, wherein the polypeptide is FXN.

523. The method of embodiment 519, wherein the polypeptide is SMN.

524. The method of embodiment 519, wherein the polypeptide is GCase.

525. The method of embodiment 519, wherein the polypeptide is N-sulfoglucosamine sulfohydrolase.

526. The method of embodiment 519, wherein the polypeptide is N-acetyl-alpha-glucosaminidase.

527. The method of embodiment 519, wherein the polypeptide is iduronate 2-sulfatase.

528. The method of embodiment 519, wherein the polypeptide is alpha-L-iduronidase.

529. The method of embodiment 519, wherein the polypeptide is palmitoyl-protein thioesterase 1.

530. The method of embodiment 519, wherein the polypeptide is tripeptidyl peptidase 1.

531. The method of embodiment 519, wherein the polypeptide is battenin.

532. The method of embodiment 519, wherein the polypeptide is CLN5.

533. The method of embodiment 519, wherein the polypeptide is CLN6 (linclin).

534. The method of embodiment 519, wherein the polypeptide is MFSD8.

535. The method of embodiment 519, wherein the polypeptide is CLN8.

536. The method of embodiment 519, wherein the polypeptide is ASPA.

537. The method of embodiment 519, wherein the polypeptide is GRN

538. The method of embodiment 519, wherein the polypeptide is MeCP2.

539. The method of embodiment 519, wherein the polypeptide is GLB1.

540. The method of embodiment 519, wherein the polypeptide is GAN.

541. The method of embodiment 519, wherein the polypeptide is ATP2A2.

542. The method of embodiment 519, wherein the polypeptide is S100A1.

543. The method of any one of embodiments 1-542, wherein the subject is a mammal.

544. The method of any one of embodiments 1-543, wherein the subject is a human.

545. The method of any of the embodiments 1-544, whereby the AAV particle is used for treatment, amelioration, or prevention of a neurological disease.

546. The method of embodiment 545, wherein the neurological disease stems from a loss or partial loss of protein or function of a protein in the subject.

547. The method of embodiment 545, wherein the neurological disease is selected from the group consisting of Parkinson's Disease (PD), Multiple System Atrophy (MSA), and Friedreich's Ataxia (FA).

548. The method of embodiment 547, wherein the neurological disease is PD.

549. The method of embodiment 547, wherein the neurological disease is MSA.

550. The method of embodiment 547, wherein the neurological disease is FA.

551. The method of embodiment 545, wherein the neurological disease stems from a gain or partial gain of function mutation in a protein in the subject.

552. The method of embodiment 551, wherein the neurological disease is selected from the group consisting of tauopathies, Alzheimer's disease (AD), Amyotrophic lateral sclerosis (ALS), Huntington's Disease (HD), and neuropathic pain.

553. The method of embodiment 552, wherein the neurological disease is tauopathy.

554. The method of embodiment 552, wherein the neurological disease is AD.

555. The method of embodiment 552, wherein the neurological disease is ALS.

556. The method of embodiment 552, wherein the neurological disease is HD.

557. The method of embodiment 552, wherein the neurological disease is neuropathic pain.

558. A method of treating Huntington's Disease, comprising: delivering at least one payload molecule to a brain region of a subject with Huntington's Disease, comprising CM administration of at least one AAV particle to cerebrospinal fluid (CSF) of the subject, wherein the at least one AAV particle comprises a viral genome that encodes at least one payload molecule, and a capsid protein, whereby the at least one payload molecule is expressed in the brain region, wherein the capsid protein serotype is selected from the group consisting of AAV1, AAV6, AAV6mt1, and AAV6mt3, the brain region is caudate, and the at least one payload molecule is a modulatory polynucleotide that suppresses or inhibits expression of HTT.

559. The method of embodiment 558, wherein the capsid protein serotype is AAV1.

560. The method of embodiment 558, wherein the capsid protein serotype is AAV6.

561. The method of embodiment 558, wherein the capsid protein serotype is AAV6mt1.

562. The method of embodiment 558, wherein the capsid protein serotype is AAV6mt3.

563. The method of embodiments 558-562, wherein the polynucleotide is an siRNA duplex.

564. A method of treating Alzheimer's Disease, comprising: delivering at least one payload molecule to at least one brain region of a subject with Alzheimer's Disease, comprising CM administration of at least one AAV particle to cerebrospinal fluid (CSF) of the subject, wherein the at least one AAV particle comprises a viral genome that encodes at least one payload molecule, and a capsid protein, whereby the at least one payload molecule is expressed in the at least one brain region, wherein the capsid protein serotype is selected from the group consisting of AAV6, AAV6mt1, and AAV6mt3, the at least one brain region is hippocampus, and the at least one payload molecule is a modulatory polynucleotide that suppresses or inhibits expression of amyloid precursor protein, microtubule-associated protein tau, or alpha synuclein.

565. The method of claim 564, wherein the capsid protein serotype is AAV6.

566. The method of claim 565, wherein the modulatory polynucleotide suppresses or inhibits expression of amyloid precursor protein.

567. The method of claim 565, wherein the modulatory polynucleotide suppresses or inhibits expression of microtubule-associated protein tau.

568. The method of claim 565, wherein the modulatory polynucleotide suppresses or inhibits expression of alpha synuclein.

569. The method of claim 564, wherein the capsid protein serotype is AAV6mt1.

570. The method of claim 569, wherein the modulatory polynucleotide suppresses or inhibits expression of amyloid precursor protein.

571. The method of claim 569, wherein the modulatory polynucleotide suppresses or inhibits expression of microtubule-associated protein tau.

572. The method of claim 569, wherein the modulatory polynucleotide suppresses or inhibits expression of alpha synuclein.

573. The method of claim 564, wherein the capsid protein serotype is AAV6mt3.

574. The method of claim 573, wherein the modulatory polynucleotide suppresses or inhibits expression of amyloid precursor protein.

575. The method of claim 573, wherein the modulatory polynucleotide suppresses or inhibits expression of microtubule-associated protein tau.

576. The method of claim 573, wherein the modulatory polynucleotide suppresses or inhibits expression of alpha synuclein.

577. The method of embodiments 564-576, wherein the polynucleotide is an siRNA duplex.

### BRIEF DESCRIPTION OF THE DRAWINGS

The foregoing and other objects, features and advantages will be apparent from the following description of particular embodiments of the disclosure, as illustrated in the accompanying drawings. The drawings are not necessarily to scale, emphasis instead being placed upon illustrating the principles of various embodiments of the disclosure.

FIG. 1A: A schematic map of a DNA-barcoded AAV genome described herein.

FIG. 1B: Illustration of the barcoded AAV library containing 58 different AAV capsids that was evaluated herein.

### DETAILED DESCRIPTION

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

### I. Adeno-Associated Virus (AAV), AAV Particle, and Capsid Protein

### ADENO-Associated Viruses (AAVs) and AAV Particles

Viruses of the Parvoviridae family are small non-enveloped icosahedral capsid viruses characterized by a single stranded DNA genome. Parvoviridae family viruses consist of two subfamilies: Parvovirinae, which infect vertebrates, and Densovirinae, which infect invertebrates. Due to its relatively simple structure, easily manipulated using standard molecular biology techniques, this virus family is useful as a biological tool. The genome of the virus may be modified to contain a minimum of components for the assembly of a functional recombinant virus, or viral particle, which is loaded with or engineered to express or deliver a desired payload, which may be delivered to a target cell, tissue, organ, or organism. The parvoviruses and other members of the Parvoviridae family are generally described in Kenneth I. Bems, “Parvoviridae: The Viruses and Their Replication,” Chapter 69 in FIELDS VIROLOGY (3d Ed. 1996), the contents of which are incorporated by reference in their entirety.

The Parvoviridae family comprises the Dependovirus genus which includes adeno-associated viruses (AAV) capable of replication in vertebrate hosts including, but not limited to, human, primate, bovine, canine, equine, and ovine species.

The AAV viral genome is a linear, single-stranded DNA (ssDNA) molecule approximately 5,000 nucleotides (nt) in length. The AAV viral genome can comprise a payload region and at least one inverted terminal repeat (ITR) or ITR region. ITRs traditionally flank the coding nucleotide sequences for the non-structural proteins (encoded by Rep genes) and the structural proteins (encoded by capsid genes or Cap genes). While not wishing to be bound by theory, an AAV viral genome typically comprises two ITR sequences. The AAV vector genome comprises a characteristic T-shaped hairpin structure defined by the self-complementary terminal 145 nt of the 5′ and 3′ ends of the ssDNA which form an energetically stable double stranded region. The double stranded hairpin structures comprise multiple functions including, but not limited to, acting as an origin for DNA replication by functioning as primers for the endogenous DNA polymerase complex of the host viral replication cell.

In addition to the encoded heterologous payload, AAV particles described herein comprise one or more capsid protein serotypes and/or sequences of Table 1 and may comprise the viral genome, in whole or in part, of any naturally occurring and/or recombinant AAV serotype nucleotide sequence or variant.

In one embodiment, AAV particles comprising one or more capsid protein serotypes of Table 1 are recombinant AAV viral vectors which are replication defective, lacking sequences encoding functional Rep and Cap proteins within their viral genome. These defective AAV particles may lack most or all parental coding sequences and essentially carry only one or two AAV ITR sequences and the nucleic acid of interest for delivery to a cell, a tissue, an organ or an organism.

In one embodiment, the viral genome of the AAV particles comprising one or more capsid protein serotypes of Table 1 for use in delivery of payloads to a central nervous system region, for example, a brain region, via administration to the CSF comprise at least one control element which provides for the replication, transcription and translation of a coding sequence encoded therein. Not all of the control elements need always be present as long as the coding sequence is capable of being replicated, transcribed and/or translated in an appropriate host cell. Non-limiting examples of expression control elements include sequences for transcription initiation and/or termination, promoter and/or enhancer sequences, efficient RNA processing signals such as splicing and polyadenylation signals, sequences that stabilize cytoplasmic mRNA, sequences that enhance translation efficacy (e.g., Kozak consensus sequence), sequences that enhance protein stability, and/or sequences that enhance protein processing and/or secretion.

In various non-limiting examples, AAV particles comprising one or more capsid protein serotypes of Table 1 can be used for delivery of payloads to a brain region, via administration to the CSF where the brain region is the frontal cortex, occipital cortex, caudate nucleus, putamen, thalamus, hippocampus, cingulate gyrus, hypothalamus, pons, medulla, cerebellar Purkinje layer, or cerebellar granular layer.

According to the present disclosure, AAV particles comprising one or more capsid protein serotypes of Table 1 for use in delivery of payloads to a central nervous system region, for example, a brain region, via administration to the CSF comprise a virus that has been distilled or reduced to the minimum components necessary for transduction of a nucleic acid payload or cargo of interest. In this manner, AAV particles comprising one or more capsid protein serotypes of Table 1 are engineered as vehicles for specific delivery while lacking the deleterious replication and/or integration features found in wild-type viruses.

AAV particles comprising one or more capsid protein serotypes of Table 1 may be produced recombinantly and may be based on adeno-associated virus (AAV) parent or reference sequences. As used herein, a “vector” is any molecule or moiety which transports, transduces or otherwise acts as a carrier of a heterologous molecule such as the nucleic acids described herein.

In addition to single stranded AAV viral genomes (e.g., ssAAVs), the present disclosure also provides for self-complementary AAV (scAAVs) viral genomes, scAAV vector genomes contain DNA strands which anneal together to form double stranded DNA. By skipping second strand synthesis, scAAVs allow for rapid expression in the cell.

In one embodiment, an AAV particle comprising one or more capsid protein serotypes of Table 1 is an scAAV.

In one embodiment, an AAV particle comprising one or more capsid protein serotypes of Table 1 is an ssAAV.

Methods for producing and/or modifying AAV particles are disclosed in the art such as pseudotyped AAV particles (PCT Patent Publication Nos. WO200028004; WO200123001; WO2004112727; WO 2005005610 and WO 2005072364, the content of each of which is incorporated herein by reference in its entirety).

In one embodiment, the AAV particles comprising one or more capsid protein serotypes of Table 1 comprise at least one payload region encoding the polypeptides or polynucleotides described herein and may be introduced into mammalian cells.

### Capsid Protein Serotypes

In one embodiment, described herein are capsid protein serotypes, or variants thereof, as found in Table 1.

In one aspect, AAV particles are described herein that comprise one or more capsid proteins, or variants thereof, described herein.

In one embodiment, a capsid protein serotype described herein may be selected from any of those capsid protein serotypes found in Table 1. In one embodiment, the capsid protein serotype may be a variant of any of the capsid protein serotypes found in Table 1. In one embodiment, AAV particles are described herein that comprise such a capsid protein or proteins, or variants thereof.

In one embodiment, described herein are polynucleotide sequences encoding the amino acid capsid protein serotypes described in Table 1. In one embodiment, the capsid protein or proteins may be encoded by a polynucleotide sequence that is a codon optimized version of a polynucleotide sequence encoding the amino acid sequence of Table 1. For example, the polynucleotide sequence is codon optimized for expression in insect cells, such as Sf9 insect cells. In one embodiment, the capsid protein or proteins may be encoded by a polynucleotide sequence that differs from the amino acid sequence of Table 1 due to amino acid code degeneracy. In one embodiment, AAV particles are described herein that comprise a capsid protein or proteins, or variants thereof, encoded by such a polynucleotide.

In any of the DNA and RNA sequences referenced and/or described herein, the single letter symbol has the following description: A for adenine; C for cytosine; G for guanine; T for thymine; U for Uracil; W for weak bases such as adenine or thymine; S for strong nucleotides such as cytosine and guanine; M for amino nucleotides such as adenine and cytosine; K for keto nucleotides such as guanine and thymine; R for purines adenine and guanine; Y for pyrimidine cytosine and thymine; B for any base that is not A (e.g., cytosine, guanine, and thymine); D for any base that is not C (e.g., adenine, guanine, and thymine); H for any base that is not G (e.g., adenine, cytosine, and thymine); V for any base that is not T (e.g., adenine, cytosine, and guanine); N for any nucleotide (which is not a gap); and Z is for zero.

In any of the amino acid sequences referenced and/or described herein, the single letter symbol has the following description: G (Gly) for Glycine; A (Ala) for Alanine; L (Leu) for Leucine; M (Met) for Methionine; F (Phe) for Phenylalanine; W (Trp) for Tryptophan; K (Lys) for Lysine; Q (Gln) for Glutamine; E (Glu) for Glutamic Acid; S (Ser) for Serine; P (Pro) for Proline; V (Val) for Valine; I (Ile) for Isoleucine C (Cys) for Cysteine Y (Tyr) for Tyrosine; H (His) for Histidine; R (Arg) for Arginine; N (Asn) for Asparagine; D (Asp) for Aspartic Acid; T (Thr) for Threonine; B (Asx) for Aspartic acid or Asparagine; J (Xle) for Leucine or Isoleucine; O (Pyl) for Pyrrolysine; U (Sec) for Selenocysteine; X (Xaa) for any amino acid; and Z (Glx) for Glutamine or Glutamic acid.

In one embodiment, AAV particles described herein comprise capsid proteins, or variants thereof, which are encoded by a polynucleotide and an RNA splice variant or variants of the polynucleotide. In one embodiment, the AAV particle comprises VP1, VP2 and VP3 capsid proteins serotypes of one or more of the serotypes as shown in Table 1, or as encoded by a polynucleotide sequence encoding the amino acid sequences in Table 1. In one embodiment of such an AAV particle, the VP1:VP2:VP3 ratio is 1-2:1:10.

TABLE 1
AAV Capsid Serotypes and Protein Sequences

| | AAV Capsid in | Amino acid |
|---|---|---|
| AAV Capsid Protein | Barcoded Library | SEQ ID NO |
| AAV1 | AAV1 | 1 |
| CLv-1 | AAV1mt1 | 2 |
| CLv-6 | AAV1mt2 | 3 |
| AAVCkd-7 | AAV1mt3 | 4 |
| AAV2 | AAV2 | 5 |
| AAV2-R585E | AAV2m1 or AAV2mt1 | 6 |
| AAV2VR1.6 | AAV2mt2 | 7 |
| AAV2VR1.5 | AAV2mt3 | 8 |
| AAV2VR4.1 | AAV2mt4 | 9 |
| AAV2VR4.5 | AAV2mt5 | 10 |
| AAV2VR4.2 | AAV2mt6 | 11 |
| AAV2VR4.4 | AAV2mt7 | 12 |
| AAV2VR4.3 | AAV2mt8 | 13 |
| AAV2VR4.6 | AAV2mt9 | 14 |
| AAV2EVEVRIV | AAV2mt10 | 15 |
| AAV3B | AAV3B | 16 |
| AAVCBr-7_2(AAV3B) | AAV3mt1 | 17 |
| AAVCBr-7_5(AAV3B) | AAV3mt2 | 18 |
| AAVCBr-7_8(AAV3B) | AAV3mt3 | 19 |
| AAVCBr-7_4(AAV3B) | AAV3mt4 | 20 |
| AAV4 | AAV4 | 21 |
| AAV5 | AAV5 | 22 |
| CBr-B7_4(AAV5) | AAV5mt1 | 23 |
| CHt-P6(AAV5) | AAV5mt2 | 24 |
| AAVCHt-6_1(AAV5) | AAV5mt3 | 25 |
| AAVCHt-6_10(AAV5) | AAV5mt4 | 26 |
| AAVCsp8_8(AAV5) | AAV5mt5 | 27 |
| AAV6 | AAV6 | 28 |
| AAV6_2 | AAV6mt1 | 29 |
| Ckd-B5(AAV6) | AAV6mt2 | 30 |
| AAVCkd-B7(AAV6) | AAV6mt3 | 31 |
| AAVCkd-B8(AAV6) | AAV6mt4 | 32 |
| CKd-H3Var2(AAV6) | AAV6mt5 | 33 |
| AAV7 | AAV7 | 34 |
| AAV8 | AAV8 | 35 |
| AAV9 | AAV9 | 36 |
| CLv1-3(AAV9) | AAV9mt1 | 37 |
| CLv-D8(AAV9) | AAV9mt2 | 38 |
| CLv-D3(AAV9) | AAV9mt3 | 39 |
| CBr-E1(AAV9) | AAV9mt4 | 40 |
| AAVCBrE4(AAV9) | AAV9mt5 | 41 |
| 79-CLv-D5(AAV9) | AAV9mt6 | 42 |
| 91-CLv-R8(AAV9) | AAV9mt7 | 43 |
| 75Var-CLv-D1(AAV9) | AAV9mt8 | 44 |
| AAVCBr-E5(AAV9) | AAV9mt9 | 45 |
| AAVClg-F1(AAV9) | AAV9mt10 | 46 |
| AAVCsp-3(AAV9) | AAV9mt11 | 47 |
| AAVCSP11(AAV9) | AAV9mt12 | 48 |
| AAV11 | AAV11 | 49 |
| AAVrh8 | AAVrh8 | 50 |
| AAVrh10 | AAVrh10 | 51 |
| AAVrh39 | AAVrh39 | 52 |
| AAVrh43 | AAVrh43 | 53 |
| AAVDJ | AAVDJ | 54 |
| AAVDJ8 | AAVDJ8 | 55 |
| AAV-Pig(Po4) | Pig | 56 |
| AAV-Mouse | Mouse | 57 |
| AAV-Avian(DA-1) | Avian | 58 |

As used herein, a first capsid protein is considered a variant of a second capsid protein if the amino acid sequence of the first capsid protein has at least 90%, 91%, 92%, 93%, 94%, 95%, 96%, 97%, 98% or 99% identity to the amino acid sequence of the second capsid protein. Differences between amino acid sequence of a capsid protein and a variant of the capsid protein can include amino acid substitutions (for example, conservative amino acid substitutions), deletions and insertions.

In one embodiment, the initiation codon for translation of the AAV VP1 capsid protein may be CTG, TTG, or GTG as described in U.S. Pat. No. 8,163,543, the contents of which are herein incorporated by reference in its entirety.

The present disclosure refers to structural capsid proteins (including VP1, VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the peptide sequence (Met), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins comprising the viral capsid may be produced, some of which may include a Met1/AA1 amino acid (Met+/AA+) and some of which may lack a Met/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 February 19. 327(5968): 973-977; the contents of which are each incorporated herein by reference in its entirety.

According to the present invention, references to capsid proteins is not limited to either clipped (Met−/AA−) or unclipped (Met+/AA+) and may, in context, refer to independent capsid proteins, viral capsids comprised of a mixture of capsid proteins, and/or polynucleotide sequences (or fragments thereof) which encode, describe, produce or result in capsid proteins of the present disclosure. A direct reference to a “capsid protein” or “capsid polypeptide” (such as VP1, VP2 or VP2) may also comprise VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific SEQ ID NO: (whether a protein or nucleic acid) which comprises or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met1” amino acid (Met+) encoded by the AUG/ATG start codon may also be understood to teach a VP polypeptide sequence which is 735 amino acids in length and which does not include the “Met1” amino acid (Met−) of the 736 amino acid Met+ sequence.

As a second non-limiting example, reference to a VP1 polypeptide sequence which is 736 amino acids in length and which includes an “AA1” amino acid (AA1+) encoded by any NNN initiator codon may also be understood to teach a VP polypeptide sequence which is 735 amino acids in length and which does not include the “AA1” amino acid (AA1−) of the 736 amino acid AA1+ sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1−).

As a non-limiting example, an AAV capsid serotype can include VP1 (Met+/AA1+), VP1 (Met−/AA−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

### Capsid Engineering and DNA Barcoding

Recombinant or engineered AAV vectors have shown promise for use in therapy for the treatment of human disease. However, a need still exists for AAV particles with more specific and/or enhanced tropism for target tissues. Capsid engineering methods have been used to try to identify capsids with enhanced transduction of target tissues (e.g., brain, spinal cord, DRG). A variety of methods have been used, including mutational methods, DNA barcoding, directed evolution, random peptide insertions, and capsid shuffling and/or chimeras.

One method described for high-throughput characterization of the phenotypes of a large number of AAV serotypes is known as AAV Barcode-Seq (Adachi K et al. Nature Communications 5:3075 (2014), the contents of which are herein incorporated by reference in their entirety). In this next-generation sequencing (NGS) based method, AAV libraries are created comprising DNA barcode tags, which can be assessed by multi-plexed Illumina barcode sequencing. This method can be used to identify AAV variants with altered receptor binding, tropism, neutralization and or blood clearance as compared to wild-type or non-variant sequences. Amino acids of the AAV capsid that are important to these functions can also be identified in this manner.

As described in Adachi et al 2014, AAV capsid libraries were generated, wherein each mutant carried a wild-type AAV2 rep gene and an AAV cap gene derived from a series of variants or mutants, and a pair of left and right 12-nucleotide long DNA bar-codes downstream of an AAV2 polyadenylation signal (pA). In this manner, 7 different DNA barcode AAV capsid libraries were generated. Capsid libraries were then provided to mice. At a pre-set timepoint, samples were collected, DNA extracted and PCR-amplified using AAV-clone specific virus bar codes and sample-specific bar code attached PCR primers. All the virus barcode PCR amplicons were Illumina sequenced and converted to raw sequence read number data by a computational algorithm. The core of the Barcode-Seq approach is a 96-nucleotide cassette comprising the two DNA bar-codes (left and right) described above, three PCR primer binding sites and two restriction enzyme sites. As an exemplar, an AAV rep-cap genome was used, but the system can be applied to any AAV viral genome, including one devoid of rep and cap genes. The advantage of the Barcode Seq method is the collection of a large data set and correlation to desirable phenotype with few replicates and in a short period of time. The DNA Barcode Seq method can be similarly applied to RNA.

In some embodiments, a DNA barcode library may be utilized to identify AAV capsids with enhanced tropism for CNS tissues. The barcodes (also referred to herein as virus barcodes (VBC)) may comprise up to 5, 6, 7, 8, 9, 10, 11, 12, 13, 14, 15, 16, 17, 18, 19, 20, 21, 22, 23, 24, or 25 nucleotides in length. The barcodes may be located downstream of a promoter (e.g. pA or U6). The barcodes may be 1, 2, 3, 4, 5, 6, 7, 8, 9, 10, 11, 12, 13, 14, 15, 20, 25, 30, 35, 40, 45, 50, 55, 60, 65, 70, 75, 80, 85 or more than 85 nucleotides downstream of the promoter.

The DNA-barcoded AAV vector genome may be single or double stranded. In some embodiments, the DNA-barcoded AAV vector genome is single stranded. In some embodiments, the DNA-barcoded AAV vector genome is double stranded.

In one embodiment, DNA barcoding may be used to identify AAVs. In one embodiment, the AAV vector genome may comprise one or more virus barcodes, as described in Davidsson et al., (Scientific Reports (2016) 6:37563.) and in Marsic et al. (Molecular Therapy—Methods and Clinical Development 2, 15041 (2015)), the contents of each of which are herein incorporated by reference in their entirety.

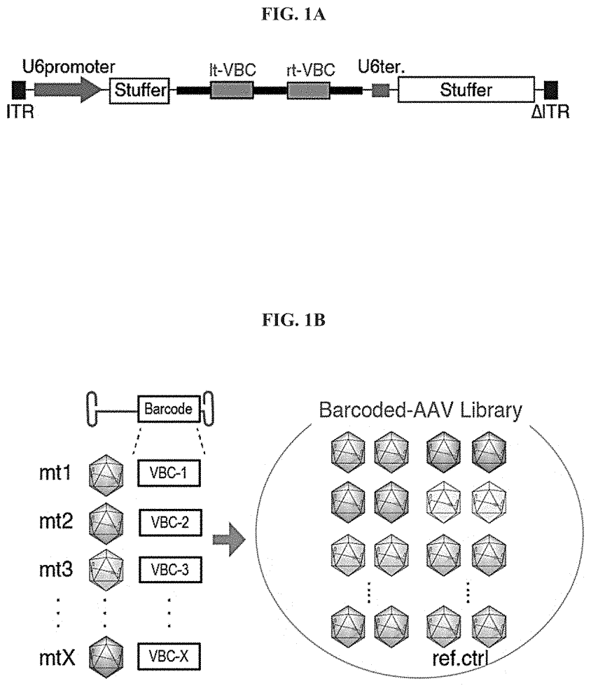

As shown in FIG. 1A, the AAV vector genome may comprise a pair of DNA barcodes. The pair of DNA virus barcodes may include a left virus barcode (It-VBC) and a right virus barcode (rt-VBC). The virus barcode pair may be independently up to 5, 6, 7, 8, 9, 10, 11, 12, 13, 14, 15, 16, 17, 18, 19, or 20 nucleotides in length. The virus barcode pair may be located downstream of a promoter (e.g., a u6 promoter). The virus barcode pair may be PCR-amplified independently as either a DNA barcode or an RNA barcode. The Barcode-Seq protocol as described in Adachi K et al. (Nat Commun 5, 3075 (2014) and Earley L F et al. Journal of Virology 91(3): e01980-16 (2017), the contents of each of which are herein incorporated by reference in their entirety) may be used to identify and/or quantify the barcoded samples in various CNS tissues.

In some embodiments, the barcoded libraries may comprise 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different AAV capsid sequences and/or serotypes. In some embodiments, the barcoded libraries may comprise at least 100, at least 1000, at least 10,000, at least 100,000, at least 1,000,000, at least 3,000,000, or at least 5,000,000 different AAV capsid sequences and/or serotypes. DNA-barcoded AAV vectors, each with a specific AAV capsid sequence and/or serotype, may be produced separately and pooled into one library.

After administration of the AAV barcoded libraries, DNA and/or RNA may be isolated from the CNS tissues of the subject. The DNA may be isolated up to 1 week, 2 week, 3 weeks, 4 weeks, 5 weeks, 6 weeks, 7 weeks, 8 weeks, 9 weeks, 6 months, or 1 year after administration of the library. The DNA and/or RNA barcodes may be analyzed via any method known to one of skill in the art. In some embodiments, barcodes may be analyzed via the Pacific Biosciences RSII Sequencer (PacBio). In some embodiments, barcodes may be analyzed via Illumina sequencing as described above.

### II. AAV Viral Genome Components

### Inverted Terminal Repeats (ITRs)

The AAV particles comprising one or more capsid protein serotypes of Table 1 for use in delivery of payloads to a central nervous system region, for example, a brain region, via administration to the CSF comprise a viral genome with at least one ITR region and a payload region. In one embodiment, the viral genome has two ITRs. These two ITRs flank the payload region at the 5′ and 3′ ends. The ITRs function as origins of replication comprising recognition sites for replication. ITRs comprise sequence regions which can be complementary and symmetrically arranged. ITRs incorporated into viral genomes described herein may be comprised of naturally occurring polynucleotide sequences or recombinantly derived polynucleotide sequences.

In one embodiment, the AAV particle comprising one or more capsid protein serotypes of Table 1 has more than one ITR. In a non-limiting example, the AAV particle has a viral genome comprising two ITRs. In one embodiment, the ITRs are of the same serotype as one another. In another embodiment, the ITRs are of different serotypes. In one embodiment both ITRs of the viral genome of the AAV particle are AAV2 ITRs.

Independently, each ITR may be about 100 to about 150 nucleotides in length. Non-limiting examples of ITR length are 102, 105, 130, 140, 141, 142, 145 nucleotides in length, and those having at least 85%, 86%, 87%, 88%, 89%, 90%, 91%, 92%, 93%, 94%, 95% or more than 95% identity thereto.

### Promoters

In one embodiment, the payload region of the viral genome comprises at least one element to enhance the transgene target specificity and expression (See e.g., Powell et al. Viral Expression Cassette Elements to Enhance Transgene Target Specificity and Expression in Gene Therapy, 2015; the contents of which are herein incorporated by reference in its entirety). Non-limiting examples of elements to enhance the transgene target specificity and expression include promoters, endogenous miRNAs, post-transcriptional regulatory elements (PREs), polyadenylation (PolyA) signal sequences and upstream enhancers (USEs), CMV enhancers and introns.

A person skilled in the art may recognize that expression of the polypeptides described herein in a target cell may require a specific promoter, including but not limited to, a promoter that is species specific, inducible, tissue-specific, or cell cycle-specific (Parr et al., Nat. Med. 3:1145-9 (1997); the contents of which are herein incorporated by reference in their entirety).

In one embodiment, the promoter is deemed to be efficient when it drives expression of the polypeptide(s) encoded in the payload region of the viral genome of the AAV particle comprising one or more capsid proteins described herein.

In one embodiment, the promoter is a promoter deemed to be efficient when it drives expression in the cell being targeted.

In one embodiment, the promoter is a promoter having a tropism for the cell being targeted.

In one embodiment, the promoter drives expression of the payload for a period of time in targeted tissues. Expression driven by a promoter may be for a period of from 1 hour up to more than 10 years. As a non-limiting example, the promoter is a weak promoter for sustained expression of a payload in nervous tissues.

In one embodiment, the promoter drives expression of the polypeptides described herein for at least 1 month up to more than 65 years.

Promoters may be naturally occurring or non-naturally occurring. Non-limiting examples of promoters include viral promoters, plant promoters and mammalian promoters. In some embodiments, the promoters may be human promoters. In some embodiments, the promoter may be truncated or mutated.

Promoters which drive or promote expression in most tissues include, but are not limited to, human elongation factor 1α-subunit (EF1α), cytomegalovirus (CMV) immediate-early enhancer and/or promoter, chicken β-actin (CBA) and its derivative CAG, p glucuronidase (GUSB), or ubiquitin C (UBC). Tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons or subtypes of neurons, astrocytes, or oligodendrocytes.

Non-limiting examples of tissue-specific expression elements for neurons include neuron-specific enolase (NSE), platelet-derived growth factor (PDGF), platelet-derived growth factor B-chain (PDGF-β), synapsin (Syn), methyl-CpG binding protein 2 (MeCP2), Ca²⁺/calmodulin-dependent protein kinase II (CaMKII), metabotropic glutamate receptor 2 (mGluR2), neurofilament light (NFL) or heavy (NFH), β-globin minigene np2, preproenkephalin (PPE), enkephalin (Enk) and excitatory amino acid transporter 2 (EAAT2) promoters. Non-limiting examples of tissue-specific expression elements for astrocytes include glial fibrillary acidic protein (GFAP) and EAAT2 promoters. A non-limiting example of a tissue-specific expression element for oligodendrocytes includes the myelin basic protein (MBP) promoter.

In one embodiment, the promoter may be less than 1 kb. The promoter may have a length of 200 up to more than 800 nucleotides.

In one embodiment, the promoter may be a combination of two or more components of the same or different starting or parental promoters such as, but not limited to, CMV and CBA. Each component may have a length of 200 up to more than 800 nucleotides. In one embodiment, the promoter is a combination of a 382 nucleotide CMV-enhancer sequence and a 260 nucleotide CBA-promoter sequence.

In one embodiment, the viral genome comprises a ubiquitous promoter. Non-limiting examples of ubiquitous promoters include CMV, CBA (including derivatives CAG, CBh, etc.), EF-1α, PGK, UBC, GUSB (hGBp), and UCOE (promoter of HNRPA2B1-CBX3).

Yu et al. (Molecular Pain 2011, 7:63; the contents of which are herein incorporated by reference in their entirety) evaluated the expression of eGFP under the CAG, EFIα, PGK and UBC promoters in rat DRG cells and primary DRG cells using lentiviral vectors and found that UBC showed weaker expression than the other 3 promoters and only 10-12% glial expression was seen for all promoters. Soderblom et al. (E. Neuro 2015; the contents of which are herein incorporated by reference in its entirety) evaluated the expression of eGFP in AAV8 with CMV and UBC promoters and AAV2 with the CMV promoter after injection in the motor cortex. Intranasal administration of a plasmid containing a UBC or EFIα promoter showed a sustained airway expression greater than the expression with the CMV promoter (See e.g., Gill et al., Gene Therapy 2001, Vol. 8, 1539-1546; the contents of which are herein incorporated by reference in their entirety). Husain et al. (Gene Therapy 2009; the contents of which are herein incorporated by reference in its entirety) evaluated an HOH construct with a hGUSB promoter, a HSV-1LAT promoter and an NSE promoter and found that the HβH construct showed weaker expression than NSE in mouse brain. Passini and Wolfe (J. Virol. 2001, 12382-12392, the contents of which are herein incorporated by reference in its entirety) evaluated the long term effects of the HβH vector following an intraventricular injection in neonatal mice and found that there was sustained expression for at least 1 year. Low expression in all brain regions was found by Xu et al. (Gene Therapy 2001, 8, 1323-1332: the contents of which are herein incorporated by reference in their entirety) when NFL and NFH promoters were used as compared to the CMV-lacZ, CMV-luc, EF, GFAP, hENK, nAChR, PPE, PPE+wpre, NSE (0.3 kb), NSE (1.8 kb) and NSE (1.8 kb+wpre). Xu et al. found that the promoter activity in descending order was NSE (1.8 kb), EF, NSE (0.3 kb), GFAP, CMV, hENK, PPE, NFL and NFH. NFL is a 650 nucleotide promoter and NFH is a 920 nucleotide promoter which are both absent in the liver but NFH is abundant in the sensory proprioceptive neurons, brain and spinal cord and NFH is present in the heart. SCN8A is a 470 nucleotide promoter which expresses throughout the DRG, spinal cord and brain with particularly high expression seen in the hippocampal neurons and cerebellar Purkinje cells, cortex, thalamus and hypothalamus (See e.g., Drews et al. Identification of evolutionary conserved, functional noncoding elements in the promoter region of the sodium channel gene SCN8A. Mamm Genome (2007) 18:723-731; and Raymond et al. Expression of Alternatively Spliced Sodium Channel α-subunit genes, Journal of Biological Chemistry (2004) 279(44) 46234-46241; the contents of each of which are herein incorporated by reference in their entireties).

Any of the promoters taught by the aforementioned Yu, Soderblom, Gill, Husain, Passini, Xu, Drews or Raymond may be used in connection with the present disclosure.

In one embodiment, the promoter is not cell specific.

In one embodiment, the promoter is an ubiquitin c (UBC) promoter. The UBC promoter may have a size of 300-350 nucleotides. As a non-limiting example, the UBC promoter is 332 nucleotides.

In one embodiment, the promoter is a β-glucuronidase (GUSB) promoter. The GUSB promoter may have a size of 350-400 nucleotides. As a non-limiting example, the GUSB promoter is 378 nucleotides.

In one embodiment, the promoter is a neurofilament light (NFL) promoter. The NFL promoter may have a size of 600-700 nucleotides. As a non-limiting example, the NFL promoter is 650 nucleotides.

In one embodiment, the promoter is a neurofilament heavy (NFH) promoter. The NFH promoter may have a size of 900-950 nucleotides. As a non-limiting example, the NFH promoter is 920 nucleotides.

In one embodiment, the promoter is a SCN8A promoter. The SCN8A promoter may have a size of 450-500 nucleotides. As a non-limiting example, the SCN8A promoter is 470 nucleotides.

In one embodiment, the promoter is a frataxin (FXN) promoter.

In one embodiment, the promoter is a phosphoglycerate kinase 1 (PGK) promoter.

In one embodiment, the promoter is a chicken β-actin (CBA) promoter.

In one embodiment, the promoter is a cytomegalovirus (CNV) promoter.

In one embodiment, the promoter is a H1 promoter.

In one embodiment, the promoter is an engineered promoter.

In one embodiment, the promoter is a liver or a skeletal muscle promoter. Non-limiting examples of liver promoters include human α-1-antitrypsin (hAAT) and thyroxine binding globulin (TBG). Non-limiting examples of skeletal muscle promoters include Desmin, MCK or synthetic C5-12.

In one embodiment, the promoter is a RNA pol III promoter. As a non-limiting example, the RNA pol III promoter is U6. As a non-limiting example, the RNA pol III promoter is H1.

In one embodiment, the viral genome comprises two promoters. As a non-limiting example, the promoters are an EF1α promoter and a CMV promoter.

In one embodiment, the viral genome comprises an enhancer element, a promoter and/or a 5′UTR intron. The enhancer element, also referred to herein as an “enhancer,” may be, but is not limited to, a CMV enhancer, the promoter may be, but is not limited to, a CMV, CBA, UBC, GUSB, NSE, Synapsin, MeCP2, and GFAP promoter and the 5′UTR/intron may be, but is not limited to. SV40, and CBA-MVM. As a non-limiting example, the enhancer, promoter and/or intron used in combination may be: (1) CMV enhancer, CMV promoter, SV40 5′UTR intron; (2) CMV enhancer, CBA promoter, SV 40 5′UTR intron; (3) CMV enhancer, CBA promoter, CBA-MVM 5′UTR intron; (4) UBC promoter; (5) GUSB promoter; (6) NSE promoter; (7) Synapsin promoter; (8) MeCP2 promoter and (9) GFAP promoter.

In one embodiment, the viral genome comprises an engineered promoter.

In another embodiment, the viral genome comprises a promoter from a naturally expressed protein.

### Untranslated Regions (UTRs)

By definition, wild type untranslated regions (UTRs) of a gene are transcribed but not translated. Generally, the 5′ UTR starts at the transcription start site and ends at the start codon and the 3′ UTR starts immediately following the stop codon and continues until the termination signal for transcription.

While not wishing to be bound by theory, wild-type 5′ untranslated regions (UTRs) include features which play roles in translation initiation. Kozak sequences, which are commonly known to be involved in the process by which the ribosome initiates translation of many genes, are usually included in 5′ UTRs. Kozak sequences have the consensus CCR(A/G)CCAUGG, where R is a purine (adenine or guanine) three bases upstream of the start codon (ATG), which is followed by another ‘G’.

In one embodiment, the 5′UTR in the viral genome includes a Kozak sequence. In one embodiment, the 5′UTR in the viral genome does not include a Kozak sequence.

While not wishing to be bound by theory, wild-type 3′ UTRs are known to have stretches of Adenosines and Uridines embedded therein. These AU rich signatures are particularly prevalent in genes with high rates of turnover. Based on their sequence features and functional properties, the AU rich elements (AREs) can be separated into three classes (Chen et al. 1995, the contents of which are herein incorporated by reference in its entirety): Class I AREs, such as, but not limited to, c-Myc and MyoD, contain several dispersed copies of an AUUUA motif within U-rich regions. Class II AREs, such as, but not limited to, GM-CSF and TNF-α, possess two or more overlapping UUAUUUA(U/A)(U/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of polynucleotides. When engineering specific polynucleotides, e.g., payload regions of viral genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In one embodiment, the 3′ UTR of the viral genome may include an oligo(dT) sequence for templated addition of a poly-A tail.

In one embodiment, the viral genome may include at least one miRNA seed, binding site or full sequence. microRNAs (or miRNA or miR) are 19-25 nucleotide noncoding RNAs that bind to the sites of nucleic acid targets and down-regulate gene expression either by reducing nucleic acid molecule stability or by inhibiting translation. A microRNA sequence comprises a “seed” region, i.e., a sequence in the region of positions 2-8 of the mature microRNA, which sequence has perfect Watson-Crick complementarity to the miRNA target sequence of the nucleic acid.

In one embodiment, the viral genome may be engineered to include, alter or remove at least one miRNA binding site, sequence or seed region.

Any UTR from any gene known in the art may be incorporated into the viral genome of the AAV particle comprising one or more capsid proteins described herein. These UTRs, or portions thereof, may be placed in the same orientation as in the gene from which they were selected or they may be altered in orientation or location. In one embodiment, the UTR used in the viral genome of the AAV particle may be inverted, shortened, lengthened, made with one or more other 5′ UTRs or 3′ UTRs known in the art. As used herein, the term “altered” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In one embodiment, the viral genome of the AAV particle comprising one or more capsid protein serotypes and/or sequences of Table 1 comprises at least one artificial UTR which is not a variant of a wild type UTR.

In one embodiment, the viral genome of the AAV particle comprising one or more capsid protein serotypes and/or sequences of Table 1 comprises UTRs which have been selected from a family of transcripts whose proteins share a common function, structure, feature or property.

### Polyadenylation Sequence

In one embodiment, the viral genome of the AAV particles comprising one or more capsid protein serotypes and/or sequences of Table 1 comprise at least one polyadenylation sequence. The viral genome of the AAV particle may comprise a polyadenylation sequence between the 3′ end of the payload coding sequence and the 5′ end of the 3′ITR.

In one embodiment, the polyadenylation sequence or “polyA sequence” may range from absent to about 500 nucleotides in length.

Introns

In one embodiment, the vector genome comprises at least one element to enhance the transgene target specificity and expression (See e.g., Powell et al. Viral Expression Cassette Elements to Enhance Transgene Target Specificity and Expression in Gene Therapy, 2015; the contents of which are herein incorporated by reference in its entirety) such as an intron. Non-limiting examples of introns include, MVM (67-97 bps), FIX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

In one embodiment, the intron or intron portion may be 100-500 nucleotides in length.

### Stuffer Sequences

In one embodiment, the viral genome comprises at least one element to improve packaging efficiency and expression, such as a stuffer sequence (also referred to herein as a filler sequence). Non-limiting examples of stuffer sequences include albumin and/or alpha-1 antitrypsin. Any known viral, mammalian, or plant sequence may be manipulated for use as a stuffer sequence.

In one embodiment, the stuffer or filler sequence may be from about 100-3500 nucleotides in length.

miRNA Binding Site

In one embodiment, the viral genome comprises at least one sequence encoding a miRNA binding site to reduce the expression of the transgene in a specific tissue. miRNAs and their abundance in different tissues are well known in the art. As a non-limiting example, a miR-122 miRNA binding site may be encoded in the viral genome to reduce the expression of the viral genome in the liver.

### Genome Size

In one embodiment, the AAV particle which comprises a payload described herein may be a single stranded or a double stranded vector genome. The size of the vector genome may be small, medium, large or the maximum size. Additionally, the vector genome may comprise a promoter and a polyA tail.

In one embodiment, the vector genome which comprises a payload described herein may be a small single stranded vector genome. A small single stranded vector genome may be 2.1 to 3.5 kb in size. Additionally, the vector genome may comprise a promoter and a polyA tail.

In one embodiment, the vector genome which comprises a payload described herein may be a small double stranded vector genome. A small double stranded vector genome may be 1.3 to 1.7 kb in size. Additionally, the vector genome may comprise a promoter and a polyA tail.

In one embodiment, the vector genome which comprises a payload described herein e.g., polynucleotide, siRNA or dsRNA, may be a medium single stranded vector genome. A medium single stranded vector genome may be 3.6 to 4.3 kb in size. Additionally, the vector genome may comprise a promoter and a polyA tail.

In one embodiment, the vector genome which comprises a payload described herein may be a medium double stranded vector genome. A medium double stranded vector genome may be 1.8 to 2.1 kb in size. Additionally, the vector genome may comprise a promoter and a polyA tail.

In one embodiment, the vector genome which comprises a payload described herein may be a large single stranded vector genome. A large single stranded vector genome may be 4.4 to 6.0 kb in size. Additionally, the vector genome may comprise a promoter and a polyA tail.

In one embodiment, the vector genome which comprises a payload described herein may be a large double stranded vector genome. A large double stranded vector genome may be 2.2 to 3.0 kb in size. Additionally, the vector genome may comprise a promoter and a polyA tail.

### III. Payloads

The AAV particles of the present disclosure comprise at least one payload region. As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide, for example, a multi-unit polypeptide, or a modulatory nucleic acid or regulatory nucleic acid. Payloads described herein typically encode polypeptides or fragments or variants thereof, or modulatory polynucleotides, e.g., miRNAs.

An RNA encoded by the payload region can, for example, include an mRNA, tRNA, rRNA, tmRNA, miRNA, siRNA, piRNA, shRNA antisense RNA, double stranded RNA, snRNA, snoRNA, or long non-coding RNA (lncRNA).

The payload region may be constructed in such a way as to reflect a region similar to or mirroring the natural organization of an mRNA.

The payload region may comprise a combination of coding and non-coding nucleic acid sequences.

In some embodiments, the AAV payload region may encode a coding or non-coding RNA. For example, an RNA encoded by the payload region can include an mRNA, tRNA, rRNA, tmRNA, miRNA, siRNA, piRNA, shRNA antisense RNA, double stranded RNA, snRNA, snoRNA, or long non-coding RNA (ncRNA).

In certain embodiments, the AAV payload region encodes one or more microRNAs (or miRNA) which are 19-25 nucleotide long noncoding RNAs that bind to the 3′UTR of nucleic acid molecules and down-regulate gene expression either by reducing nucleic acid molecule stability or by inhibiting translation. The payload region can include one or more microRNA target sequences, microRNA sequences, or microRNA seeds. Such sequences can correspond to any known microRNA such as those taught in US Publication No. US2005/0261218 and US Publication No. US2005/0059005, the contents of each of which are incorporated herein by reference in their entirety. A microRNA sequence includes a seed region, i.e., a sequence in the region of positions 2-8 of the mature microRNA, which has perfect Watson-Crick complementarity to the miRNA target sequence. A microRNA seed can include positions 2-8 or 2-7 of the mature microRNA. In some embodiments, a microRNA seed can include 7 nucleotides (e.g., nucleotides 2-8 of the mature microRNA), wherein the seed-complementary site in the corresponding miRNA target is flanked by an adenine (A) opposed to microRNA position 1. In some embodiments, a microRNA seed can include 6 nucleotides (e.g., nucleotides 2-7 of the mature microRNA), wherein the seed-complementary site in the corresponding miRNA target is flanked by an adenine (A) opposed to microRNA position 1. See for example, Grimson A, Farh K K, Johnston W K, Garrett-Engele P, Lim L P, Bartel D P; Mol Cell. 2007 Jul. 6; 27(1):91-105; each of which is herein incorporated by reference in their entirety. The bases of the microRNA seed have complete complementarity with the target sequence.

In one embodiment, the payload region comprises more than one nucleic acid sequence encoding more than one payload molecule of interest. In one embodiment, the AAV particle comprises a viral genome with a payload region comprising nucleic acid sequences encoding more than one polypeptide of interest. In such an embodiment, a viral genome encoding more than one polypeptide may be replicated and packaged into a viral (e.g., an AAV) particle comprising one or more capsid proteins as described herein. A target cell transduced with such a viral particle comprising more than one polypeptide may express each of the polypeptides in a single cell.

In one embodiment, the payload region may comprise the additional or alternative components as described herein. At the 5′ and/or the 3′ end of the payload region, there may be at least one inverted terminal repeat (ITR). In one embodiment, within the payload region, there is a promoter region, an intron region and a coding region.

Where the AAV particle payload region encodes a polypeptide, the polypeptide may be a peptide or protein. As a non-limiting example, the payload region may encode at least one allele of apolipoprotein E (APOE) such as, but not limited to ApoE2, ApoE3 and/or ApoE4. As a second non-limiting example, the payload region may encode a human or a primate frataxin protein, or fragment or variant thereof. As another non-limiting example, the payload region may encode an antibody, or a fragment thereof. The AAV viral genomes encoding polypeptides described herein may be useful in the fields of human disease, viruses, infections, veterinary applications and a variety of in vivo and in vitro settings.

In some embodiments. AAV particles comprising one or more capsid protein serotypes and/or sequences of Table 1 may be used in delivery of payloads to a central nervous system region, for example, a brain region, via administration to the CSF in the field of medicine for the treatment, prophylaxis, palliation or amelioration of neurological diseases and/or disorders. In various non-limiting examples, AAV particles comprising one or more capsid protein serotypes and/or sequences of Table 1 can be used in the delivery of payloads to a brain region via administration to the CSF, where the brain region is the frontal cortex, occipital cortex, caudate nucleus, putamen, thalamus, hippocampus, cingulate gyrus, hypothalamus, pons, medulla, cerebellar Purkinje layer, or cerebellar granular layer, and the use is for treatment, prophylaxis, palliation or amelioration of neurological diseases and/or disorders.

In some embodiments, AAV particles comprising one or more capsid protein serotypes and/or sequences of Table 1 may be used in delivery of payloads to a central nervous system region, for example, a brain region, via administration to the CSF are useful in the field of medicine for the treatment, prophylaxis, palliation or amelioration of tauopathy. In various non-limiting examples, AAV particles comprising one or more capsid protein serotypes and/or sequences of Table 1 can be used for delivery of payloads to a brain region via administration to the CSF for treatment, prophylaxis, palliation or amelioration of tauopathies, where the brain region is the frontal cortex, occipital cortex, caudate nucleus, putamen, thalamus, hippocampus, cingulate gyrus, hypothalamus, pons, medulla, cerebellar Purkinje layer, or cerebellar granular layer.

In some embodiments, AAV particles comprising one or more capsid protein serotypes and/or sequences of Table 1 may be used in delivery of payloads to a central nervous system region, for example, a brain region, via administration to the CSF are useful in the field of medicine for the treatment, prophylaxis, palliation or amelioration of Alzheimer's Disease. In various non-limiting examples, AAV particles comprising one or more capsid protein serotypes and/or sequences of Table 1 can be used to deliver payloads to a brain region via administration to the CSF for treatment, prophylaxis, palliation or amelioration of Alzheimer's Disease, where the brain region is the frontal cortex, occipital cortex, caudate nucleus, putamen, thalamus, hippocampus, cingulate gyrus, hypothalamus, pons, medulla, cerebellar Purkinje layer, or cerebellar granular layer.

In some embodiments, AAV particles comprising one or more capsid protein serotypes and/or sequences of Table 1 may be used in delivery of payloads to a central nervous system region, for example, a brain region, via administration to the CSF are useful in the field of medicine for the treatment, prophylaxis, palliation or amelioration of Friedreich's ataxia, or any disease stemming from a loss or partial loss of frataxin protein. In various non-limiting examples, AAV particles comprising one or more capsid protein serotypes and/or sequences of Table 1 can be used for delivery of payloads to a brain region, via administration to the CSF for treatment, prophylaxis, palliation or amelioration of Friedreich's ataxia, or any disease stemming from a loss or partial loss of frataxin protein, where the brain region is the frontal cortex, occipital cortex, caudate nucleus, putamen, thalamus, hippocampus, cingulate gyrus, hypothalamus, pons, medulla, cerebellar Purkinje layer, or cerebellar granular layer.

In some embodiments, AAV particles comprising one or more capsid protein serotypes and/or sequences of Table 1 for use in delivery of payloads to a central nervous system region, for example, a brain region, via administration to the CSF are useful in the field of medicine for the treatment, prophylaxis, palliation or amelioration of Parkinson's Disease. In various non-limiting examples, AAV particles comprising one or more capsid protein serotypes and/or sequences of Table 1 can be used for delivery of payloads to a brain region, via administration to the CSF for treatment, prophylaxis, palliation or amelioration of Parkinson's Disease, where the brain region is the frontal cortex, occipital cortex, caudate nucleus, putamen, thalamus, hippocampus, cingulate gyrus, hypothalamus, pons, medulla, cerebellar Purkinje layer, or cerebellar granular layer.

In some embodiments, AAV particles comprising one or more capsid protein serotypes and/or sequences of Table 1 for use in delivery of payloads to a central nervous system region, for example, a brain region, via administration to the CSF are useful in the field of medicine for the treatment, prophylaxis, palliation or amelioration of Amyotrophic lateral sclerosis. In various non-limiting examples, AAV particles comprising one or more capsid protein serotypes and/or sequences of Table 1 can be used for delivery of payloads to a brain region, via administration to the CSF for treatment, prophylaxis, palliation or amelioration of Amyotrophic lateral sclerosis, where the brain region is the frontal cortex, occipital cortex, caudate nucleus, putamen, thalamus, hippocampus, cingulate gyrus, hypothalamus, pons, medulla, cerebellar Purkinje layer, or cerebellar granular layer.

In some embodiments, AAV particles comprising one or more capsid protein serotypes and/or sequences of Table 1 for use in delivery of payloads to a central nervous system region, for example, a brain region, via administration to the CSF are useful in the field of medicine for the treatment, prophylaxis, palliation or amelioration of Huntington's Disease. In various non-limiting examples, AAV particles comprising one or more capsid protein serotypes and/or sequences of Table 1 can be used for delivery of payloads to a brain region, via administration to the CSF for treatment, prophylaxis, palliation or amelioration of Huntington's Disease, where the brain region is the frontal cortex, occipital cortex, caudate nucleus, putamen, thalamus, hippocampus, cingulate gyrus, hypothalamus, pons, medulla, cerebellar Purkinje layer, or cerebellar granular layer.

Amino acid sequences encoded by payload regions of the viral genomes described herein may be translated as a whole polypeptide, a plurality of polypeptides or fragments of polypeptides, which independently may be encoded by one or more nucleic acids, fragments of nucleic acids or variants of any of the aforementioned. As used herein, “polypeptide” means a polymer of amino acid residues (natural or unnatural) linked together most often by peptide bonds. The term, as used herein, refers to proteins, polypeptides, and peptides of any size, structure, or function. In some instances, the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

Sequence tags or amino acids, such as one or more lysines, can be added to the peptide sequences described herein (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

### Nucleic Acids Encoding a Protein of Interest

In one embodiment, the payload region of the AAV particle comprising one or more capsid protein serotypes and/or sequences as shown in Table 1, comprises one or more nucleic acid sequences encoding a protein of interest. In some embodiments, the protein of interest is an antibody, an antibody fragment, antibody variant, Aromatic L-Amino Acid Decarboxylase (AADC), APOE2, Frataxin (FXN), survival motor neuron (SMN) protein, glucocerebrosidase (GCase), N-sulfoglucosamine sulfohydrolase, N-acetyl-alpha-glucosaminidase, iduronate 2-sulfatase, alpha-L-iduronidase, palmitoyl-protein thioesterase 1, tripeptidyl peptidase 1, battenin, CLN5, CLN6 (linclin), MFSD8, CLN8, aspartoacylase (ASPA), progranulin (GRN), MeCP2, beta-galactosidase (GLB1), gigaxonin (GAN), ATPase Sarcoplasmic/Endoplasmic Reticulum Ca2+ Transporting 2 (ATP2A2), and S100 Calcium Binding Protein A1 (S100A1).

### Apolipoproten E (APOE)

In one embodiment, the payload region of the AAV particle comprises one or more nucleic acid sequences encoding an allele of the apolipoprotein E (APOE) gene (e.g., ApoE2, ApoE3, and/or ApoE4), for example, an allele of the human APOE gene. In a non-limiting example, the payload region of the AAV particle comprises a nucleic acid sequence, or fragment thereof, as found at NCBI reference numbers NP_00032.1, NP_001289618.1, NP_0, NP_001289617.1, NM_000041.3, NM_001302689.1, NM_001302690.1, or NM_001302688.1, or Ensembl reference numbers ENSP00000252486, ENSP000413135, ENSP00000413653, ENSP00000410423, ENST0000252486.8, ENST0000044699.5, ENST0000045628.2, ENST00000434152.5, or ENST00000425718.1.

### Frataxin (PFUV)

In one embodiment, the payload region of the AAV particle comprises one or more nucleic acid sequences encoding frataxin (FXN) for example, human frataxin. In a non-limiting example, the payload region of the AAV particle comprises a nucleic acid sequence, or fragment thereof, as found at NCBI reference numbers NP_000135.2, NP_852090.1, NP_001155178.1, NM_000144.4, NM_181425.2, or NM_001161706.1.

### Aromatic L-Amino Acid Decarboxylases (AADC)

In one embodiment, the payload region of the AAV particle comprises one or more nucleic acid sequences encoding Aromatic L-Amino Acid Decarboxylase (AADC), for example, human AADC. In a non-limiting example, the payload region of the AAV particle comprises a nucleic acid sequence, or fragment thereof, as found at NCBI reference numbers NP_00078.1 or NM_000790.3.

### Antibody

In one embodiment, the payload region of the AAV particle comprises one or more nucleic acid sequences encoding the heavy chain and/or light chain of an antibody directed against a tau protein, for example, a human tau protein. In one embodiment, the ta antibody is the Paired Helical Filamentous 1 (PHF-1) antibody.

### Modulatory Polynucleotides as Payloads

In one embodiment, the present disclosure relates to AAV particles comprising one or more capsid protein serotypes and/or sequences as shown in Table 1, wherein the AAV particles encode modulatory polynucleotides, e.g., RNA or DNA molecules, as therapeutic agents that can suppress or inhibit expression of a gene of interest. In one embodiment, a gene of interest is superoxide dismutase 1 (SOD1), chromosome 9 open reading frame 72 (C9ORF72), TAR DNA binding protein (TARDBP), ataxin 3 (ATXN3), huntingtin (HTT), amyloid precursor protein (APP), apolipoprotein E (APOE), microtubule-associated protein tau (MAPT), alpha synuclein (SNCA), voltage-gated sodium channel alpha subunit 9 (SCN9A), and voltage-gated sodium channel alpha subunit 10 (SCN10A).

RNA interference mediated gene silencing, for example, can specifically inhibit targeted gene expression. The present disclosure then provides small double stranded RNA (dsRNA) molecules (small interfering RNA, siRNA) targeting a gene of interest, pharmaceutical compositions comprising such siRNAs, as well as processes of their design. The present disclosure also provides methods of their use for inhibiting gene expression and protein production of a gene of interest, for treating a neurological disease.

In one embodiment, the present disclosure provides small interfering RNA (siRNA) duplexes (and modulatory polynucleotides encoding them) that target the mRNA of a gene of interest to interfere with the gene expression and/or protein production.

In one embodiment, the siRNA duplexes described herein may target the gene of interest along any segment of their respective nucleotide sequence.

In one embodiment, the siRNA duplexes described herein may target the gene of interest at the location of a SNP or variant within the nucleotide sequence.

In some embodiments, a nucleic acid sequence encoding such siRNA molecules, or a single strand of the siRNA molecules, is inserted into adeno-associated viral vectors and introduced into cells, specifically cells in the central nervous system, for example, a brain region.

AAV particles have been investigated for siRNA delivery because of several unique features. Non-limiting examples of the features include (i) the ability to infect both dividing and non-dividing cells; (ii) a broad host range for infectivity, including human cells; (iii) wild-type AAV has not been associated with any disease and has not been shown to replicate in infected cells; (iv) the lack of cell-mediated immune response against the vector and (v) the non-integrative nature in a host chromosome thereby reducing potential for long-term expression. Moreover, infection with AAV particles has minimal influence on changing the pattern of cellular gene expression (Stilwell and Samulski et al., Biotechniques, 2003, 34, 148).

siRNA duplex sequences generally contain an antisense strand and a sense strand hybridized together forming a duplex structure, wherein the antisense strand is complementary to the nucleic acid sequence of the targeted gene, and wherein the sense strand is homologous to the nucleic acid sequence of the targeted gene. In some aspects, the 5′end of the antisense strand has a 5′ phosphate group and the 3′end of the sense strand contains a 3′hydroxyl group. In other aspects, there are zero, one or 2 nucleotide overhangs at the 3′end of each strand.

In one aspect, each strand of the siRNA duplex targeting a gene of interest is about 19 to 25, 19 to 24 or 19 to 21 nucleotides in length, preferably about 19 nucleotides, 20 nucleotides, 21 nucleotides, 22 nucleotides, 23 nucleotides, 24 nucleotides, or 25 nucleotides in length. In some aspects, the siRNAs may be unmodified RNA molecules.

In other aspects, the siRNAs may contain at least one modified nucleotide, such as base, sugar or backbone modification.

In one embodiment, an siRNA or dsRNA includes at least two sequences that are complementary to each other. The dsRNA includes a sense strand having a first sequence and an antisense strand having a second sequence. The antisense strand includes a nucleotide sequence that is substantially complementary to at least part of an mRNA encoding the target gene, and the region of complementarity is 30 nucleotides or less, and at least 15 nucleotides in length. Generally, the dsRNA is 19 to 25, 19 to 24 or 19 to 21 nucleotides in length. In some embodiments, the dsRNA is from about 15 to about 25 nucleotides in length, and in other embodiments the dsRNA is from about 25 to about 30 nucleotides in length.

The dsRNA, whether directly administered or encoded in an expression vector upon contacting with a cell expressing the target protein, inhibits the expression of the protein by at least 10%, at least 20%, at least 25%, at least 30%, at least 35% or at least 40% or more, such as when assayed by a method as described herein.

The siRNA molecules included in the compositions featured herein comprise a dsRNA having an antisense strand (the antisense strand) having a region that is 30 nucleotides or less, generally 19 to 25, 19 to 24 or 19 to 21 nucleotides in length, that is substantially complementary to at least part of an mRNA transcript of the target gene.

In one aspect, AAV particles described herein comprise one or more capsid protein serotypes and/or sequences of Table 1 and a vector genome comprising nucleic acids that encode siRNA duplexes. For example, in one embodiment, such an AAV particle comprises one or more of the capsid protein serotypes and/or sequences in Table 1, or variants thereof.

In one aspect, the siRNA molecules are designed and tested for their ability in reducing target gene mRNA levels in cultured cells.

The present disclosure also provides pharmaceutical compositions comprising an AAV particle comprising one or more capsid protein serotypes and/or sequences of Table 1 for use in delivery of payloads to a central nervous system region, for example, a brain region, via administration to the CSF and a viral genome that encodes at least one siRNA duplex targeting a gene of interest and a pharmaceutically acceptable carrier.

In one embodiment, an siRNA duplex encoded by an AAV particle comprising one or more capsid protein serotypes and/or sequences of Table 1 may be used to reduce the expression of a target protein and/or mRNA in at least one region of the CNS. The expression of target protein and/or mRNA can, for example, be reduced by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in at least one region of the CNS. As a non-limiting example, the expression of target protein and mRNA in the neurons (e.g., cortical neurons) is reduced by 50-90%. As a non-limiting example, the expression of target protein and mRNA in the neurons (e.g., cortical neurons) is reduced by 40-50%.

In some embodiments, the present disclosure provides methods for treating, or ameliorating neurological disorders associated with a target gene and/or target protein in a subject in need of treatment, the method comprising administering to the subject a pharmaceutically effective amount of an AAV particle comprising one or more capsid protein serotypes and/or sequences of Table 1 that encodes at least one siRNA duplex targeting the gene of interest, delivering said particle to targeted cells, inhibiting target gene expression and protein production, and ameliorating symptoms of a neurological disorder in the subject.

In some embodiments, an AAV particle comprising one or more capsid protein serotypes and/or sequences of Table 1 and comprising a nucleic acid sequence encoding at least one siRNA duplex targeting a gene of interest is administered to the subject in need for treating and/or ameliorating a neurological disorder. The AAV particle can comprise one or more capsid protein serotypes and/or sequences in Table 1 or variants thereof.

In some embodiments, AAV particles comprising one or more capsid protein serotypes and/or sequences of Table 1 and comprising a nucleic acid encoding such siRNA molecules may be introduced directly into the central nervous system of the subject. In some embodiments, this introduction may be via infusion into the CSF of a subject.

In some embodiments, a pharmaceutical composition described herein is used as a solo therapy. In other embodiments, a pharmaceutical composition described herein is used in combination therapy. The combination therapy may be in combination with one or more neuroprotective agents such as small molecule compounds, growth factors and hormones which have been tested for their neuroprotective effect on motor neuron degeneration.

In some embodiments, the present disclosure provides methods for treating, or ameliorating a neurological disorder, whether manifesting peripherally (PNS) or centrally (CNS) by administering to a subject in need thereof a therapeutically effective amount of an AAV particle comprising one or more capsid protein serotypes and/or sequences of Table 1 and one or more nucleic acid sequences encoding a selected payload (e.g., an siRNA molecule) described herein.

### Target Genes

Non-limiting examples of the neurological diseases which may be treated by administration of AAV particles comprising one or more capsid protein serotypes and/or sequences of Table 1 for use in delivery of payloads to a central nervous system region, for example, a brain region, via administration to the CSF wherein the AAV particles encode one or more modulatory polynucleotides described herein include tauopathies, Alzheimer Disease, Huntington's Disease, and/or Amyotrophic Lateral Sclerosis. Target genes may be any of the genes associated with any neurological disease such as, but not limited to, those listed herein.

In one embodiment, the target gene is an allele of the apolipoprotein E (APOE) gene (e.g., ApoE2, ApoE3, and/or ApoE4), for example, an allele of human APOE. In another embodiment, the target gene is superoxide dismutase (SOD1), for example, human SOD1. In one non-limiting example, the SOD1 target gene has a sequence as found at NCBI reference number NM_00454.4. In another embodiment, the target gene is huntingtin (HTT), for example, human HT. As a non-limiting example, the HTT target gene has a sequence as found at NCBI reference number NM_002111.7. As another non-limiting example, the HTT target gene is HTT and the target gene encodes an amino acid sequence as found at NCBI reference number NP_002102.4.

In yet another embodiment, the target gene is microtubule-associated protein tau (MAPT). As a non-limiting example, the target gene is MAPT and the target gene has a sequence of any of the nucleic acid sequences or amino acid sequences found at NCBI reference numbers NP_058519.3, NP_005901.2, NP_058518.1, NP_058525.1, NP_001116539.1, NP_001116538.2, NP_001190180.1, NP_001190181.1. NM_016835.4, NM_005910.5, NM_016834.4, NM_016841.4. NM_001123067.3, NM_001123066.3, NM_001203251.1, or NM_001203252.1.

Some guidelines for designing siRNAs have been proposed in the art. These guidelines generally recommend generating a 19-nucleotide duplexed region, symmetric 2-3 nucleotide 3′ overhangs, 5-phosphate and 3-hydroxyl groups targeting a region in the gene to be silenced. Other rules that may govern siRNA sequence preference include, but are not limited to, (i) A/U at the 5′ end of the antisense strand; (ii) G/C at the 5′ end of the sense strand; (iii) at least five A/U residues in the 5′ terminal one-third of the antisense strand; and (iv) the absence of any GC stretch of more than 9 nucleotides in length. In accordance with such consideration, together with the specific sequence of a target gene, highly effective siRNA molecules essential for suppressing mammalian target gene expression may be readily designed.

In one aspect, siRNA molecules (e.g., siRNA duplexes or encoded dsRNA) that target a gene of interest are designed. Such siRNA molecules can specifically suppress target gene expression and protein production. In some aspects, the siRNA molecules are designed and used to selectively “knock out” target gene variants in cells, i.e., transcripts that are identified in neurological disease. In some aspects, the siRNA molecules are designed and used to selectively “knock down” target gene variants in cells.

In one embodiment, an siRNA molecule described herein comprises a sense strand and a complementary antisense strand in which both strands are hybridized together to form a duplex structure. The antisense strand has sufficient complementarity to the target mRNA sequence to direct target-specific RNAi. i.e., the siRNA molecule has a sequence sufficient to trigger the destruction of the target mRNA by the RNAi machinery or process.

In some embodiments, the antisense strand and target mRNA sequences have 100% complementarity. The antisense strand may be complementary to any part of the target mRNA sequence.

In other embodiments, the antisense strand and target mRNA sequences comprise at least one mismatch. As a non-limiting example, the antisense strand and the target mRNA sequence have at least 30%, 40%, 50%, 60%, 70%, 80%, 81%, 82%, 830, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementary.

In one aspect, the siRNA molecule has a length from about 10-50 or more nucleotides, i.e., each strand comprising 10-50 nucleotides (or nucleotide analogs). Preferably, the siRNA molecule has a length from about 15-30, e.g., 15, 16, 17, 18, 19, 20, 21, 22, 23, 24, 25, 26, 27, 28, 29, or 30 nucleotides in each strand, wherein one of the strands is sufficiently complementary to a target region. In one embodiment, the siRNA molecule has a length from about 19 to 25, 19 to 24 or 19 to 21 nucleotides.

In one embodiment, the siRNA molecules described herein may comprise an antisense sequence and a sense sequence, or a fragment or variant thereof. As a non-limiting example, the antisense sequence and the sense sequence have at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementary.

### Molecular Scaffold

In one embodiment, described herein are AAV particles comprising one or more capsid proteins described herein, wherein the AAV particles encode the siRNA molecules in a modulatory polynucleotide which also comprises a molecular scaffold. As used herein a “molecular scaffold” is a framework or starting molecule that forms the sequence or structural basis against which to design or make a subsequent molecule.

In one embodiment, the modulatory polynucleotide which comprises the payload (e.g., siRNA, miRNA or other RNAi agent described herein) includes a molecular scaffold which comprises a leading 5′ flanking sequence which may be of any length and may be derived in whole or in part from wild type microRNA sequence or be completely artificial. A 3′ flanking sequence may mirror the 5′ flanking sequence in size and origin. Either flanking sequence may be absent. The 3′ flanking sequence may optionally contain one or more CNNC motifs, where “N” represents any nucleotide.

In some embodiments, one or both of the 5′ and 3′ flanking sequences are absent.

In some embodiments the 5′ and 3′ flanking sequences are the same length.

In some embodiments the 5′ flanking sequence is from 1-10 nucleotides in length, from 5-15 nucleotides in length, from 10-30 nucleotides in length, from 20-50 nucleotides in length, greater than 40 nucleotides in length, greater than 50 nucleotides in length, greater than 100 nucleotides in length or greater than 200 nucleotides in length.

In some embodiments, the 5′ flanking sequence may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 nucleotides in length.

In some embodiments the 3′ flanking sequence is from 1-10 nucleotides in length, from 5-15 nucleotides in length, from 10-30 nucleotides in length, from 20-50 nucleotides in length, greater than 40 nucleotides in length, greater than 50 nucleotides in length, greater than 100 nucleotides in length or greater than 200 nucleotides in length.

In some embodiments, the 3′ flanking sequence may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 nucleotides in length.

In some embodiments the 5′ and 3′ flanking sequences are the same sequence. In some embodiments they differ by 2%, 3%, 4%, 5%, 10%, 20% or more than 30% when aligned to each other.

Forming the stem of a stem loop structure is a minimum of at least one payload sequence. In some embodiments, the payload sequence comprises at least one nucleic acid sequence which is in part complementary or will hybridize to the target sequence. In some embodiments, the payload is an siRNA molecule or fragment of an siRNA molecule.

In some embodiments, the 5′ arm of the stem loop comprises a sense sequence.

In some embodiments, the 3′ arm of the stem loop comprises an antisense sequence. The antisense sequence, in some instances, comprises a “G” nucleotide at the 5′ most end.

In other embodiments, the sense sequence may reside on the 3′ arm while the antisense sequence resides on the 5′ arm of the stem of the stem loop structure.

The sense and antisense sequences may be completely complementary across a substantial portion of their length. In other embodiments, the sense sequence and antisense sequence may be at least 70, 80, 90, 95 or 99% complementary across independently at least 50, 60, 70, 80, 85, 90, 95, or 99% of the length of the strands.

Neither the identity of the sense sequence nor the homology of the antisense sequence need be 100% complementary to the target.

Separating the sense and antisense sequence of the stem loop structure is a loop (also known as a loop motif). The loop may be of any length, between 4-30 nucleotides, between 4-20 nucleotides, between 4-15 nucleotides, between 5-15 nucleotides, between 6-12 nucleotides, 6 nucleotides, 7, nucleotides, 8 nucleotides, 9 nucleotides, 10 nucleotides, 11 nucleotides, and/or 12 nucleotides.

In some embodiments, the loop comprises at least one UGUG motif. In some embodiments, the UGUG motif is located at the 5′ terminus of the loop.

Spacer regions may be present in the modulatory polynucleotide to separate one or more modules from one another. There may be one or more such spacer regions present.

In one embodiment, a spacer region of between 8-20, i.e., 8, 9, 10, 11, 12, 13, 14, 15, 16, 17, 18, 19, or 20 nucleotides may be present between the sense sequence and a flanking sequence.

In one embodiment, the spacer is 13 nucleotides and is located between the 5′ terminus of the sense sequence and a flanking sequence. In one embodiment, a spacer is of sufficient length to form approximately one helical turn of the sequence.

In one embodiment, a spacer region of between 8-20, i.e., 8, 9, 10, 11, 12, 13, 14, 15, 16, 17, 18, 19, or 20 nucleotides may be present between the antisense sequence and a flanking sequence.

In one embodiment, the spacer sequence is between 10-13, i.e., 10, 11, 12 or 13 nucleotides and is located between the 3′ terminus of the antisense sequence and a flanking sequence. In one embodiment, a spacer is of sufficient length to form approximately one helical turn of the sequence.

In one embodiment, the modulatory polynucleotide comprises in the 5′ to 3′ direction, a 5′ flanking sequence, a 5′ arm, a loop motif, a 3′ arm and a 3′ flanking sequence. As a non-limiting example, the 5′ arm may comprise a sense sequence and the 3′ arm comprises the antisense sequence. In another non-limiting example, the 5′ arm comprises the antisense sequence and the 3′ arm comprises the sense sequence.

In one embodiment, the 5′ arm, payload (e.g., sense and/or antisense sequence), loop motif and/or 3′ arm sequence may be altered (e.g., substituting 1 or more nucleotides, adding nucleotides and/or deleting nucleotides). The alteration may cause a beneficial change in the function of the construct (e.g., increase knock-down of the target sequence, reduce degradation of the construct, reduce off target effect, increase efficiency of the payload, and reduce degradation of the payload).

In one embodiment, the molecular scaffold of the modulatory polynucleotides is aligned in order to have the rate of excision of the guide strand be greater than the rate of excision of the passenger strand. The rate of excision of the guide or passenger strand may be, independently, 1%, 2%, 3%, 4%, 5%, 10%, 15%, 20%, 25%, 30%, 35%, 40%45%, 50%, 55%, 60%, 65%, 70%, 75%, 80%, 85%, 90%, 95%, 99% or more than 99%. As a non-limiting example, the rate of excision of the guide strand is at least 80%. As another non-limiting example, the rate of excision of the guide strand is at least 90%.

In one embodiment, the rate of excision of the guide strand is greater than the rate of excision of the passenger strand. In one aspect, the rate of excision of the guide strand may be at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99% or more than 99% greater than the passenger strand.

In one embodiment, the efficiency of excision of the guide strand is at least 60%, 65%, 70%, 75%, 80%, 85%, 90%, 95%, 99% or more than 99%. As a non-limiting example, the efficiency of the excision of the guide strand is greater than 80%.

In one embodiment, the efficiency of the excision of the guide strand is greater than the excision of the passenger strand from the molecular scaffold. The excision of the guide strand may be 2, 3, 4, 5, 6, 7, 8, 9, 10 or more than 10 times more efficient than the excision of the passenger strand from the molecular scaffold.

In one embodiment, the molecular scaffold comprises a dual-function targeting modulatory polynucleotide. As used herein, a “dual-function targeting” modulatory polynucleotide is a polynucleotide where both the guide and passenger strands knock down the same target or the guide and passenger strands knock down different targets.

In one embodiment, the molecular scaffold of the modulatory polynucleotides described herein comprise a 5′ flanking region, a loop region and a 3′ flanking region.

In one embodiment, the molecular scaffold may comprise one or more linkers known in the art. The linkers may separate regions or one molecular scaffold from another. As a non-limiting example, the molecular scaffold may be polycistronic.

In one embodiment, the modulatory polynucleotide is designed using at least one of the following properties: loop variant, seed mismatch/bulge/wobble variant, stem mismatch, loop variant and basal stem mismatch variant, seed mismatch and basal stem mismatch variant, stem mismatch and basal stem mismatch variant, seed wobble and basal stem wobble variant, or a stem sequence variant.

In one embodiment, AAV particles comprising one or more capsid protein subtypes and/or sequences of Table 1 for use in delivery of payloads to a central nervous system region, for example, a brain region, via administration to the CSF may be introduced into cells which are relevant to the disease to be treated. As a non-limiting example, the disease is a tauopathy and/or Alzheimer's Disease and the target cells are entorhinal cortex, hippocampal or cortical neurons. In various non-limiting examples, AAV particles comprising one or more capsid protein serotypes and/or sequences of Table 1 can be used for delivery of payloads to a brain region, via administration to the CSF where the brain region is the frontal cortex, occipital cortex, caudate nucleus, putamen, thalamus, hippocampus, cingulate gyrus, hypothalamus, pons, medulla, cerebellar Purkinje layer, or cerebellar granular layer for treatment, prophylaxis, palliation or amelioration of diseases.

In one embodiment, AAV particles comprising one or more capsid protein serotypes and/or sequences of Table 1 may be introduced into cells which have a high level of endogenous expression of the target sequence.

In another embodiment, AAV particles comprising one or more capsid protein serotypes and/or sequences of Table 1 may be introduced into cells which have a low level of endogenous expression of the target sequence.

In one embodiment, the cells may be those which have a high efficiency of AAV transduction.

In other embodiments, AAV particles comprising one or more capsid protein serotypes and/or sequences of Table 1 and comprising a nucleic acid sequence encoding the siRNA molecules described herein may be used to deliver siRNA molecules to the central nervous system, for example, into a brain region.

In one embodiment, an AAV particle comprising one or more capsid protein serotypes and/or sequences of Table 1 that comprises a nucleic acid sequence encoding siRNA molecules described herein may encode siRNA molecules which are polycistronic molecules. The siRNA molecules may additionally comprise one or more linkers between regions of the siRNA molecules.

In one embodiment, an AAV particle comprising one or more capsid protein serotypes and/or sequences of Table 1 for use in delivery of payloads to a central nervous system region, for example, a brain region, via administration to the CSF and comprising a nucleic acid sequence encoding a payload of interest (e.g., one expressing or targeting Frataxin, APOE, Tau) described herein may be formulated for CNS delivery.

In one embodiment, an AAV particle comprising one or more capsid protein serotypes and/or sequences of Table 1 and comprising a nucleic acid sequence encoding an siRNA molecule described herein may be administered directly to the CNS. As a non-limiting example, the vector comprises a nucleic acid sequence encoding a siRNA molecule targeting ApoE, for example, ApoE2, ApoE3, or ApoE4. As a non-limiting example, the vector comprises a nucleic acid sequence encoding an siRNA molecule targeting SOD1. As a non-limiting example, the vector comprises a nucleic acid sequence encoding an siRNA molecule targeting HT. As a non-limiting example, the vector comprises a nucleic acid sequence encoding an siRNA molecule targeting Tau.

### IV. Delivery to the CNS

In one aspect, presented herein are methods of delivering a payload molecule to a central nervous system region of a subject, comprising administering an AAV vector to cerebrospinal fluid (CSF) of the subject, wherein the AAV vector comprises a viral genome that encodes the payload molecule and a capsid protein, whereby the payload molecule is expressed in the central nervous system region. In another aspect, presented herein are methods of delivering a payload molecule to a brain region of a subject, comprising administering an AAV vector to cerebrospinal fluid (CSF) of the subject, wherein the AAV vector comprises a viral genome that encodes the payload molecule and a capsid protein, whereby the payload molecule is expressed in the brain region.

In one embodiment the capsid protein is a capsid protein serotype and/or sequence shown in Table 1. In another embodiment, the capsid protein serotype is selected from the group consisting of CLv-1, CLv-6, AAVCkd-7, AAV2-R585E, AAV2VR1.6, AAV2VR1.5, AAV2VR4.1, AAV2VR4.5, AAV2VR4.2, AAV2VR4.4, AAV2VR4.3, AAV2VR4.6, AAV2EVEVRIV, AAVCBr-7_2(AAV3B), AAVCBr-7_5(AAV3B), AAVCBr-7_8(AAV3B), AAVCBr-7_4(AAV3B), CBr-B87_4(AAV5), CHt-P6(AAV5), AAVCHt-6_1(AAV5), AAVCHt-6_10(AAV5), AAVCsp8_8(AAV5), AAV6_2, Ckd-B5(AAV6), AAVCkd-B7(AAV6), AAVCkd-B8(AAV6), CKd-H3Var2(AAV6), CLv1-3(AAV9), CLv-D8(AAV9). CLv-D3(AAV9), CBr-E1(AAV9), AAVCBrE4(AAV9), 79-CLv-D5(AAV9), 91-CLv-R8(AAV9), 75Var-CLv-D1(AAV9), AAVCBr-E5(AAV9), AAVClg-F1(AAV9), AAVCsp-3(AAV9), AAVCSP11(AAV9), AAV11, AAVrh8, AAVrh10, AAVrh39, AAVrh43, AAVDJ, and AAVDJ8.

In one embodiment, delivery of payloads by adeno-associated virus (AAV) particles to cells of the central nervous system region, for example, brain region, comprises infusion into cerebrospinal fluid (CSF). CSF is produced by specialized ependymal cells that comprise the choroid plexus located in the ventricles of the brain. CSF produced within the brain then circulates and surrounds the central nervous system including the brain and spinal cord. CSF continually circulates around the central nervous system, including the ventricles of the brain and subarachnoid space that surrounds both the brain and spinal cord, while maintaining a homeostatic balance of production and reabsorption into the vascular system. The entire volume of CSF is replaced approximately four to six times per day or approximately once every four hours, though values for individuals may vary.

In various non-limiting examples, AAV particles comprising one or more capsid protein serotypes and/or sequences of Table 1 can be used for delivery of payloads to a brain region, via administration to the CSF where the brain region is the frontal cortex, occipital cortex, caudate nucleus, putamen, thalamus, hippocampus, cingulate gyrus, hypothalamus, pons, medulla, cerebellar Purkinje layer, or cerebellar granular layer.

In one embodiment, the AAV particles may be delivered by a route to bypass the liver metabolism.

In one embodiment, the AAV particles may be delivered to reduce degradation of the AAV particles and/or degradation of the formulation in the blood.

In one embodiment, the AAV particles may be delivered to bypass anatomical blockages such as, but not limited to, the blood brain barrier.

In one embodiment, the AAV particles may be formulated and delivered to a subject by a route which increases the speed of drug effect as compared to oral delivery.

In one embodiment, the AAV particles may be delivered by a method to provide uniform transduction of the spinal cord and dorsal root ganglion (DRG). As a non-limiting example, the AAV particles may be delivered using intrathecal infusion such that administration is via CSF. The intrathecal infusion may be a bolus infusion or it may be a continuous infusion. As another non-limiting example, the AAV particles are delivered using continuous intrathecal infusion over a period of about 10 hours.

In one embodiment, the AAV particles may be delivered to a subject via a single route administration.

In one embodiment, the AAV particles may be delivered to a subject via a multi-site route of administration. For example, a subject may be administered the AAV particles at 2, 3, 4, 5 or more than 5 sites.

In one embodiment, the AAV particles may be formulated. As a non-limiting example, the baricity and/or osmolarity of the formulation may be optimized to ensure optimal drug distribution in the central nervous system region, for example, a brain region.

In one embodiment, a subject may be administered the AAV particles described herein via CSF using a catheter. The catheter may be placed in the lumbar region or the cervical region of a subject. As a non-limiting example, the catheter may be placed in the lumbar region of the subject. As another non-limiting example, the catheter may be placed in the cervical region of the subject. As yet another non-limiting example, the catheter may be placed in the high cervical region of the subject. As used herein, the “high cervical region” refers to the region of the spinal cord comprising the cervical vertebrae C1, C2, C3 and C4 or any subset thereof.

In one embodiment, a subject may be administered the AAV particles described herein using a bolus infusion. As used herein, a “bolus infusion” means a single and rapid infusion of a substance or composition.

In one embodiment, a subject may be administered the AAV particles described herein using sustained delivery over a period of minutes, hours or days. The infusion rate may be changed depending on the subject, distribution, formulation or another delivery parameter known to those in the art.

In one embodiment, the intracranial pressure may be evaluated prior to administration. The route, volume, AAV particle concentration, infusion duration and/or vector titer may be optimized based on the intracranial pressure of a subject.

In one embodiment, the AAV particles described herein may be delivered by a method which allows even distribution of the AAV particles along the CNS taking into account cerebrospinal fluid (CSF) dynamics. While not wishing to be bound by theory, CSF turnover (TO) occurs approximately 6 times/day or every 4 hours and thus continuous delivery of the AAV particles at a fixed rate, may lead to AAV particles which have distributed throughout the CNS.

In one embodiment, AAV particles are delivered taking into account the oscillating movement of the CSF around the spinal cord. Vortexes are formed by the oscillating movement of the CSF around the cord and these individual vortices combine to form vortex arrays. The arrays combine to form fluid paths for movement of the AAV particles along the spinal cord.

In one embodiment, the delivery method and duration is chosen to provide broad transduction in the spinal cord. As a non-limiting example, intrathecal delivery is used to provide broad transduction along the rostral-caudal length of the spinal cord. As another non-limiting example, multi-site infusions provide a more uniform transduction along the rostral-caudal length of the spinal cord. As yet another non-limiting example, prolonged infusions provide a more uniform transduction along the rostral-caudal length of the spinal cord.

In one embodiment, delivery of AAV particles comprising a viral genome encoding a payload described herein to sensory neurons in the dorsal root ganglion (DRG), ascending spinal cord sensory tracts, and cerebellum will lead to an increased expression of the encoded payload. The increased expression may lead to improved survival and function of various cell types.

In one embodiment, delivery of AAV particles comprising a nucleic acid sequence encoding frataxin to sensory neurons in the dorsal root ganglion (DRG), ascending spinal cord sensory tracts, and cerebellum leads to an increased expression of frataxin. The increased expression of frataxin then leads to improved survival, ataxia (balance) and gait, sensory capability, coordination of movement and strength, functional capacity and quality of life and/or improved function of various cell types.

In one embodiment, the AAV particles may be delivered by injection into the CSF pathway. Non-limiting examples of delivery to the CSF pathway include intrathecal and intracerebroventricular administration.

In one embodiment, the AAV particles may be delivered by direct injection into CSF of brain ventricles. As a non-limiting example, the brain delivery may be by intrastriatal administration.

In one embodiment, delivery of AAV particles to cells of the central nervous system, is performed by intracerebroventricular (ICV) prolonged infusion. In one embodiment, delivery of AAV particles to cells of the central nervous system, for example a brain region is performed by intracerebroventricular (ICV) prolonged infusion into the CSF surrounding the parenchyma. ICV prolonged infusion comprises delivery by injection into the ventricular system of the brain. ICV prolonged infusion may comprise delivery to any of the ventricles of the brain, including, but not limited to, either of the two lateral ventricles left and right, third ventricle, and/or fourth ventricle. ICV prolonged infusion may comprise delivery to any of the foramina, or channels that connect the ventricles, including, but not limited to, interventricular foramina, also called the foramina of Monroe, cerebral aqueduct, and/or central canal. ICV prolonged infusion may comprise delivery to any of the apertures of the ventricular system including, but not limited to, the median aperture (aka foramen of Magendie), right lateral aperture, and/or left lateral aperture (aka foramina of Lushka). In one embodiment, ICV prolonged infusion comprises delivery to the perivascular space in the brain.

In one embodiment, delivery of AAV particles to cells of the central nervous system, for example, into a brain region, is performed by intrathecal (IT) prolonged infusion. In one embodiment, delivery of AAV particles to cells of a brain region is performed by intrathecal (IT) prolonged infusion. IT prolonged infusion comprises delivery by injection into the subarachnoid space, between the arachnoid membrane and pia mater, which comprises the channels through which CSF circulates. IT prolonged infusion comprises delivery to any area of the subarachnoid space including, but not limited to, perivascular space and the subarachnoid space along the entire length of the spinal cord and surrounding the brain.

In one embodiment, delivery of AAV particles to cells of the central nervous system is performed by intrathecal (IT) prolonged infusion into the spinal cord. Spinal cord segments, regions and their numbering are shown in Table 2.

TABLE 2
Spinal cord segments in human, cynomolgus and rhesus monkeys

| Spinal Cord | | Cynomolgus | Rhesus |
|---|---|---|---|
| Region | Human | Monkey | Monkey |
| Cervical | C1-C7 | C1-C7 | C1-C7 |
| Thoracic | T1-T12 | T1-T12 | T1-T12 |
| Lumbar | L1-L5 | L1-L7 | L1-L7 |
| Sacral | S1-S5 | S1-S3 | S1-S3 |
| Coccygeal (caudal) | Co1 | Co1-3 | Co1-3 |

Additionally, the spinal cord can also be divided into six regions anatomically and functionally (Sengul et al., 2013 (Sengul, G., Watson, C., Tanaka. I., Paxinos, G., 2013. Atlas of the Spinal Cord of the Rat, Mouse, Marmoset, Rhesus, and Human. Elsevier Academic Press, San Diego), and also Watson et al., Neuroscience Research 93:164-175 (2015)). These regions are the neck muscle region, the upper limb muscle region, the sympathetic outflow region, the lower limb muscle region, the parasympathetic outflow region, and the tail muscle region. These six regions also correlate with territories defined by gene expression during development (see, e.g., Watson et al., supra). The six regions can be defined histologically by the presence or absence of 2 features, the lateral motor column (LMC) and the preganglionic (intermediolateral) column (PGC) (Watson et al., 2015, incorporated herein by reference in its entirety). The limb enlargements are characterized by the presence of a lateral motor column (LMC) and the autonomic regions containing a preganglionic column (PGC). The neck (prebrachial) and tail (caudal) regions have neither an LMC nor a PGC. The limb enlargements and the sympathetic outflow region are marked by particular patterns of hox gene expression in the mouse and chicken, further supporting the division of the spinal cord into these functional regions. Table 3 maps the C, T, L, S and Co designations described in Table 2 to the functional regions according to Sengul et al. and Watson et al. and maps the functional equivalents for Human, Rhesus Monkey, and Japanese Monkey (another macaque). Note: S in Rhesus Monkey and L7 in Japanese monkey is located in both crural and postcrural regions.

TABLE 3
Spinal cord regions and sections by function

| Spinal Cord Region | Human | Rhesus Monkey | Japanese Monkey |
|---|---|---|---|
| Neck Muscle Region | C1-C4 (according to | C1-C4 (according to | C1-C3 (as described in |
| (prebrachial region) | Bruce) | Sengul et al.) | Watson et al.) |
| | C1-C3 (according to | | |
| | Sengul et al.) | | |
| Upper limb Region | C5-T1 (according to | C5-T1 (according to | C4-C8 (as described in |
| (brachial region) | Bruce) | Sengul et al.) | Watson et al.) |
| | C4-T1 (according to | | |
| | Sengul et al.) | | |
| Sympathetic outflow | T2-L1 (according to | T2-L3 (according to | T1-L2 (as described in |
| region (postbrachial | Bruce) | Sengul et al.) | Watson et al.) |
| region) | T2-L1 (according to | | |
| | Sengul et al.) | | |
| Lower limb muscle | L2-S2 (according to | L4-S1 (according to | L3-L7 (as described in |
| region (crural region) | Bruce) | Sengul et al.) | Watson et al.) |
| | L3-S2 (according to | | |
| | Sengul et al.) | | |
| Parasympathetic outflow | S3-S4 (according to | S1-S3 (according to | L7-S3 (as described in |
| region (postcrural | Bruce) | Sengul et al.) | Watson et al.) |
| region) | S3-S5 (according to | | |
| | Sengul et al.) | | |
| Tail muscle region | S5-Co1 (according to | Co1-Co3 (according to | Co1-Co3 (as described |
| (caudal region) | Bruce) | Sengul et al.) | in Watson et al.) |
| | Co1 (according to Sengul | | |
| | et al.) | | |

In one embodiment, the catheter for intrathecal delivery may be located in the cervical region. The AAV particles may be delivered in a continuous or bolus infusion.

In one embodiment, the catheter for intrathecal delivery may be located in the lumbar region. The AAV particles may be delivered in a continuous or bolus infusion.

In one embodiment, if continuous delivery of the AAV particles is used, the continuous infusion may be for 1 hour, 2, hours, 3 hours, 4 hours, 5 hours, 6 hours, 7 hours, 8 hours, 9 hours, 10 hours, I1 hours, 12 hours, 13 hours, 14 hours, 15 hours, 16 hours, 17 hours, 18 hours, 19 hours, 20 hours, 21 hours, 22 hours, 23 hours, 24 hours or more than 24 hours.

In one embodiment, the catheter may be in located at one site in the spine for delivery. As a non-limiting example, the location may be in the cervical or the lumbar region. The AAV particles may be delivered in a continuous or bolus infusion.

In one embodiment, the catheter may be located at more than one site in the spine for multi-site delivery. The AAV particles may be delivered in a continuous and/or bolus infusion. Each site of delivery may be a different dosing regimen or the same dosing regimen may be used for each site of delivery. As a non-limiting example, the sites of delivery may be in the cervical and the lumbar region. As another non-limiting example, the sites of delivery may be in the cervical region. As another non-limiting example, the sites of delivery may be in the lumbar region.

In one embodiment, a subject may be analyzed for spinal anatomy and pathology prior to delivery of the AAV particles described herein comprising a capsid protein serotype and/or sequence of Table 1. As a non-limiting example, a subject with scoliosis may have a different dosing regimen and/or catheter location compared to a subject without scoliosis.

In one embodiment, the orientation of the spine subject during delivery of the AAV particles may be vertical to the ground.

In another embodiment, the orientation of the spine of the subject during delivery of the AAV particles may be horizontal to the ground.

In one embodiment, the spine of the subject may be at an angle as compared to the ground during the delivery of the AAV particles subject. The angle of the spine of the subject as compared to the ground may be at least 10, 20, 30, 40, 50, 60, 70, 80, 90, 100, 110, 120, 130, 140, 150 or 180 degrees.

In one embodiment, a subject may be delivered the AAV particles herein using two or more delivery routes. As a non-limiting example, the delivery routes may be intrathecal administration and intracerebroventricular administration.

In one embodiment, a subject may be delivered the AAV particles herein at more than one site. As a non-limiting example, the delivery may be a multi-site intrathecal delivery using a bolus injection.

In one embodiment, a subject may be delivered the AAV particles described herein comprising a capsid protein serotype and/or sequence of Table 1 by intrathecal delivery in the lumbar region via a 10 hour bolus injection.

In one embodiment, subjects such as mammals (e.g., non-human primates (NHPs)) are administered by intrathecal (IT) or intracerebroventricular (ICV) infusion the AAV particles described herein. The AAV particles may comprise scAAV or ssAAV and any of the capsid protein serotypes and/or sequences of Table 1, comprising a payload (e.g., a transgene).

In some embodiments IT prolonged infusion comprises delivery to the cervical, thoracic, and or lumbar regions of the spine. As used herein, IT prolonged infusion into the spine is defined by the vertebral level at the site of prolonged infusion. In some embodiments IT prolonged infusion comprises delivery to the cervical region of the spine at any location including, but not limited to C1, C2, C3, C4, C5, C6, C7, and/or C8. In some embodiments IT prolonged infusion comprises delivery to the thoracic region of the spine at any location including, but not limited to T1, T2, T3, T3, T4, T5, T6, T7, T8, T9, T10, T11, and/or T12. In some embodiments IT prolonged infusion comprises delivery to the lumbar region of the spine at any location including, but not limited to L1, L2, L3, L3, L4, L5, and/or L6. In some embodiments IT prolonged infusion comprises delivery to the sacral region of the spine at any location including, but not limited to S1, S2, S3, S4, or S5. In some embodiments, delivery by IT prolonged infusion comprises one or more than one site of prolonged infusion.

In some embodiments, delivery by IT prolonged infusion may comprise 1, 2, 3, 4, 5, 6, 7, 8, 9, 10, 11, 12, 13, 14, 15, 16, 17, 18, 19, 20, 21, 22, 23, 24, or 25 sites of prolonged infusion. In one embodiment, delivery by IT prolonged infusion comprises at least three sites of prolonged infusion. In one embodiment, delivery by IT prolonged infusion consists of three sites of prolonged infusion. In one embodiment, delivery by IT prolonged infusion comprises three sites of prolonged infusion at C1, T1, and L1.

In one embodiment, intrathecal administration delivers AAV particles to targeted regions of the CNS. Non-limiting examples of regions of the CNS to deliver AAV particles include dorsal root ganglion, dentate nucleus-cerebellum and the auditory pathway.

### Infusion Parameters and Volume

In some embodiments, infusion volume, duration of infusion, infusion pattems and rates for delivery of AAV particles to cells of the central nervous system, for example, into a brain region, may be determined and regulated. In one embodiment, delivery of AAV particles to cells of the central nervous system, for example, into a brain region, comprises infusion of up to 1 mL. The infusion may be at least 0.1 mL, 0.2 mL, 0.3 mL, 0.4 mL, 0.5 mL, 0.6 mL, 0.7 mL, 0.8 mL, 0.9 mL, 1 mL or the infusion may be 0.1-0.2 mL, 0.1-0.3 mL, 0.1-0.4 mL, 0.1-0.5 mL, 0.1-0.6 mL, 0.1-0.7 mL, 0.1-0.8 mL, 0.1-0.9 mL, 0.1-1 mL, 0.2-0.3 mL, 0.2-0.4 mL, 0.2-0.5 mL, 0.2-0.6 mL, 0.2-0.7 mL, 0.2-0.8 mL, 0.2-0.9 mL, 0.2-1 mL, 0.3-0.4 mL, 0.3-0.5 mL, 0.3-0.6 mL, 0.3-0.7 mL, 0.3-0.8 mL, 0.3-0.9 mL, 0.3-1 mL, 0.4-0.5 mL, 0.4-0.6 mL, 0.4-0.7 mL, 0.4-0.8 mL, 0.4-0.9 mL, 0.4-1 mL, 0.5-0.6 mL, 0.5-0.7 mL, 0.5-0.8 mL, 0.5-0.9 mL, 0.5-1 mL, 0.6-0.7 mL, 0.6-0.8 mL, 0.6-0.9 mL, 0.6-1 mL, 0.7-0.8 mL, 0.7-0.9 mL, 0.7-1 mL, 0.8-0.9 mL, 0.8-1 mL, or 0.9-1 mL.

In one embodiment, delivery of AAV particles to cells of the central nervous system, for example, a brain region, comprises infusion of between about 1 mL to about 120 mL. The infusion may be 1-5 mL, 1-10 mL, 1-15 mL, 1-20 mL, 1-25 mL, 1-30 mL, 1-35 mL, 1-40 mL, 1-45 mL, 1-50 mL, 1-55 mL, 1-60 mL, 1-65 mL, 1-70 mL, 1-75 mL, 1-80 mL, 1-85 mL, 1-90 mL, 1-95 mL, 1-100 mL, 1-105 mL, 1-110 mL, 1-115 mL, 1-120 mL, 5-10 mL, 5-15 mL, 5-20 mL, 5-25 mL, 1-30 mL, 5-35 mL, 5-40 mL, 5-45 mL, 5-50 mL, 5-55 mL, 5-60 mL, 5-65 mL, 5-70 mL, 5-75 mL, 5-80 mL, 5-85 mL, 5-90 mL, 5-95 mL, 5-100 mL, 5-105 mL, 5-110 mL, 5-115 mL, 1-120 mL, 10-15 mL, 10-20 mL, 10-25 mL, 10-30 mL, 10-35 mL, 10-40 mL, 10-45 mL, 10-50 mL, 10-55 mL, 10-60 mL, 10-65 mL, 10-70 mL, 10-75 mL, 10-80 mL, 10-85 mL, 10-90 mL, 10-95 mL, 10-100 mL, 10-105 mL, 10-110 mL, 10-115 mL, 10-120 mL 15-20 mL, 15-25 mL, 15-30 mL, 15-35 mL, 15-40 mL, 15-45 mL, 15-50 mL, 15-55 mL, 15-60 mL, 15-65 mL, 15-70 mL, 15-75 mL, 15-80 mL, 15-85 mL, 15-90 mL, 15-95 mL, 15-100 mL, 15-105 mL, 15-110 mL, 15-115 mL, 15-120 mL, 20-25 mL, 20-30 mL, 20-35 mL, 20-40 mL, 20-45 mL, 20-50 mL, 20-55 mL, 20-60 mL, 20-65 mL, 20-70 mL, 20-75 mL, 20-80 mL, 20-85 mL, 20-90 mL, 20-95 mL, 20-100 mL, 20-105 mL, 20-110 mL, 20-115 mL, 20-120 mL, 25-30 mL, 25-35 mL, 25-40 mL, 25-45 mL, 25-50 mL, 25-55 mL, 25-60 mL, 25-65 mL, 25-70 mL, 25-75 mL, 25-80 mL, 25-85 mL, 25-90 mL, 25-95 mL, 25-100 mL, 25-105 mL, 25-110 mL, 25-115 mL, 25-120 mL, 30-35 mL, 30-40 mL, 30-45 mL, 30-50 mL, 30-55 mL, 30-60 mL, 30-65 mL, 30-70 mL, 30-75 mL, 30-80 mL, 30-85 mL, 30-90 mL, 30-95 mL, 30-100 mL, 30-105 mL, 30-110 mL, 30-115 mL, 30-120 mL, 35-40 mL, 35-45 mL, 35-50 mL, 35-55 mL, 35-60 mL, 35-65 mL, 35-70 mL, 35-75 mL, 35-80 mL, 35-85 mL, 35-90 mL, 35-95 mL, 35-100 mL, 35-105 mL, 35-110 mL, 35-115 mL, 35-120 mL, 40-45 mL, 40-50 mL, 40-55 mL, 40-60 mL, 40-65 mL, 40-70 mL, 40-75 mL, 40-80 mL, 40-85 mL, 40-90 mL, 40-95 mL, 40-100 mL, 40-105 mL, 40-110 mL, 40-115 mL, 40-120 mL, 45-50 mL, 45-55 mL, 45-60 mL, 45-65 mL, 45-70 mL, 45-75 mL, 45-80 mL, 45-85 mL, 45-90 mL, 45-95 mL, 45-100 mL, 45-105 mL, 45-110 mL, 45-115 mL, 45-120 mL, 50-55 mL, 50-60 mL, 50-65 mL, 50-70 mL, 50-75 mL, 50-80 mL, 50-85 mL, 50-90 mL, 50-95 mL, 50-100 mL, 50-105 mL, 50-110 mL, 50-115 mL, 50-120 mL, 55-60 mL, 55-65 mL, 55-70 mL, 55-75 mL, 55-80 mL, 55-85 mL, 55-90 mL, 55-95 mL, 55-100 mL, 55-105 mL, 55-110 mL, 55-115 mL, 55-120 mL, 60-65 mL, 60-70 mL, 60-75 mL, 60-80 mL, 60-85 mL, 60-90 mL, 60-95 mL, 60-100 mL, 60-105 mL, 60-110 mL, 60-115 mL, 60-120 mL, 65-70 mL, 65-75 mL, 65-80 mL, 65-85 mL, 65-90 mL, 65-95 mL, 65-100 mL, 65-105 mL, 65-110 mL, 65-115 mL, 65-120 mL, 70-75 mL, 70-80 mL, 70-85 mL, 70-90 mL, 70-95 mL, 70-100 mL, 70-105 mL, 70-110 mL, 70-115 mL, 70-120 mL, 75-80 mL, 75-85 mL, 75-90 mL, 75-95 mL, 75-100 mL, 75-105 mL, 75-110 mL, 75-115 mL, 75-120 mL, 80-85 mL, 80-90 mL, 80-95 mL, 80-100 mL, 80-105 mL, 80-110 mL, 80-115 mL, 80-120 mL, 85-90 mL, 85-95 mL, 85-100 mL, 85-105 mL, 85-110 mL, 85-115 mL, 85-120 mL, 90-95 mL, 90-100 mL, 90-105 mL, 90-110 mL, 90-115 mL, 90-120 mL, 95-100 mL, 95-105 mL, 95-110 mL, 95-115 mL, 95-120 mL, 100-105 mL, 100-110 mL, 100-115 mL, 100-120 mL, 105-110 mL, 105-115 mL, 105-120 mL, 110-115 mL, 110-120 mL, or 115-120 mL.

In one embodiment, delivery of AAV particles to cells of the central nervous system, for example, a brain region, may comprise an infusion of about 0, 1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 mL. In one embodiment, delivery of AAV particles to cells of the central nervous system, for example, a brain region, comprises of infusion of 1 mL.

In one embodiment, delivery of AAV particles to cells of the central nervous system, for example, a brain region, comprises infusion of at least 1 mL. In one embodiment, delivery of AAV particles to cells of the central nervous system, for example, a brain region, comprises infusion of at least 3 mL. In one embodiment, delivery of AAV particles to cells of the central nervous system, for example, a brain region, comprises infusion of 3 mL. In one embodiment, delivery of AAV particles to cells of the central nervous system, for example, a brain region, comprises infusion of at least 10 mL. In one embodiment, delivery of AAV particles to cells of the central nervous system, for example, a brain region, consists of infusion of 10 mL.

In one embodiment, the serotype of the AAV particles described herein may depend on the desired distribution, transduction efficiency and cellular targeting required. As described by Sorrentino et al. (comprehensive map of CNS transduction by eight adeno-associated virus serotypes upon cerebrospinal fluid administration in pigs, Molecular Therapy accepted article preview online 7 Dec. 2015; doi:10.1038/mt.2015.212; the contents of which are herein incorporated by reference in its entirety), AAV serotypes provided different distributions, transduction efficiencies and cellular targeting. In order to provide the desired efficacy, the AAV serotype needs to be selected that best matches not only the cells to be targeted but also the desired transduction efficiency and distribution.

### Duration of Infusion: Bolus Infusion

In one embodiment, delivery of AAV particles to cells of the central nervous system, for example, a brain region, comprises infusion by bolus injection with a duration of less than 30 minutes. In one embodiment, infusion by bolus injection comprises injection with a duration of less than 20 minutes. In one embodiment, infusion by bolus injection comprises injection with a duration of less than 10 minutes. In one embodiment, infusion by bolus injection comprises injection with a duration of less than 10 seconds. In one embodiment, infusion by bolus injection comprises injection with a duration of between 10 seconds to 10 minutes. In one embodiment, infusion by bolus injection comprises injection with a duration of 10 minutes. In one embodiment, infusion by bolus injection consists of injection with a duration of 10 minutes.

In one embodiment, delivery of AAV particles to cells of the central nervous system, for example, a brain region, comprises infusion by at least one bolus injection. In one embodiment, delivery may comprise infusion by 1, 2, 3, 4, 5, 6, 7, 8, 9, or 10 bolus injections. In one embodiment, delivery may comprise infusion by at least three bolus injections. In one embodiment, delivery comprises infusion by three bolus injections. In one embodiment, delivery of AAV to cells of the central nervous system, for example, a brain region, consists of infusion by three bolus injections.

In one embodiment, delivery of AAV particles to cells of the central nervous system, for example, a brain region, comprising infusion of more than one bolus injection further comprises an interval of at least one hour between injections. In one embodiment, delivery of AAV particles to cells of the central nervous system, for example, a brain region, comprising infusion of more than one bolus injection may further comprise an interval of 1, 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, 72, 78, 84, 90, 96, 108, or 120 hour(s) between injections. In one embodiment, delivery comprising infusion of more than one bolus injection further comprises an interval of one hour between injections. In one embodiment, delivery consists of infusion by three bolus injections at an interval of one hour.

In one embodiment, DRG and/or cortical brain expression may be higher with shorter, high concentration infusions.

Prolonged Infusion

In one embodiment, delivery of AAV particles to cells of the central nervous system, for example, a brain region, comprises prolonged infusion of pharmaceutically acceptable composition comprising AAV particles over a duration of at least 10 minutes. In one embodiment, delivery comprises prolonged infusion over a duration of between 30 minutes and 60 minutes. In one embodiment, delivery may comprise prolonged infusion over a duration of 0.17, 0.33, 0.5, 0.67, 0.83, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or 125 hour(s). In one embodiment, delivery comprises prolonged infusion over a duration of one hour. In one embodiment, delivery of AAV particles to cells of the central nervous system, for example, a brain region, consists of prolonged infusion over a duration of one hour.

In one embodiment, delivery of AAV particles to cells of the central nervous system, for example, a brain region, comprises prolonged infusion over a duration of 10 hours. In one embodiment, delivery of AAV particles to cells of the central nervous system, for example, a brain region, consists of prolonged infusion over a duration of 10 hours. In one embodiment, prolonged infusion may yield more homogenous levels of protein expression across the spinal cord, as compared to bolus dosing at one or multiple sites. In one embodiment, dentate nucleus expression may increase with prolonged infusions.

### Single and Multiple Rounds of Dosing

In one embodiment, delivery of AAV particles to cells of the central nervous system, for example, a brain region, comprises prolonged infusion of at least one dose. In one embodiment, delivery comprises prolonged infusion of one dose. In one embodiment, delivery of AAV to cells of the central nervous system, for example, a brain region, may comprise prolonged infusion of 1, 2, 3, 4, 5, 6, 7, 8, 9, or 10 dose(s).

### Interval of Dosing

In one embodiment, delivery of AAV particles to cells of the central nervous system, for example, a brain region, comprising prolonged infusion of more than one dose further comprises an interval of at least one hour between doses. In one embodiment, delivery may comprise an interval of 1, 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, 72, 78, 84, 90, 96, 108, or 120 hour(s) between doses. In one embodiment, delivery comprises an interval of 24 hours between doses. In one embodiment, delivery consists of three prolonged infusion doses at an interval of 24 hours.

### Infusion Patterns

### Simple (Constant)

In one embodiment, delivery of AAV particles to cells of the central nervous system, for example, a brain region, may comprise a constant rate of prolonged infusion. As used herein, a “constant rate” is a rate that stays about the same during the prolonged infusion.

### Ramped

In one embodiment, delivery of AAV particles to cells of the central nervous system, for example, a brain region, may comprise a ramped rate of prolonged infusion where the rate either increases or decreases over time. As a non-limiting example, the rate of prolonged infusion increases over time. As another non-limiting example, the rate of prolonged infusion decreases over time.

### Complex

In one embodiment, delivery of AAV particles to cells of the central nervous system, for example, a brain region, may comprise a complex rate of prolonged infusion wherein the rate of prolonged infusion alternates between high and low rates of prolonged infusion over time.

### Prolonged Infusion Rate

In one embodiment, delivery of AAV particles to cells of the central nervous system, for example, a brain region, comprises a rate of delivery, which may be defined by [VG/hour=mL/hour*VG/mL] wherein VG is viral genomes, VG/mL is composition concentration, and mL/hour is rate of prolonged infusion.

In one embodiment, delivery of AAV to cells of the central nervous system, for example, a brain region, may comprise a rate of prolonged infusion between about 0.1 mL/hour and about 25.0 mL/hour (or higher if CSF pressure does not increase to dangerous levels). In some embodiments, delivery may comprise a rate of prolonged infusion of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5.3.6, 3.7, 3.8, 3.9, 4.0, 4.1, 4.2 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8. 24.9, or 25.0 mL/hour. In some embodiments, delivery may comprise a rate of prolonged infusion of about 10, 20 30, 40, or 50 mL/hr. In one embodiment, delivery of AAV particles to cells of the central nervous system, for example, a brain region, comprises a rate of prolonged infusion of 1.0 mL/hour. In one embodiment, delivery consists of a rate of prolonged infusion of 1.0 mL/hour. In one embodiment, delivery of AAV to cells of the central nervous system, for example, a brain region, comprises a rate of prolonged infusion of 1.5 mL/hour. In one embodiment, delivery of AAV particles to cells of the central nervous system, for example, a brain region, consists of a rate of prolonged infusion of 1.5 mL/hour.

### Prolonged Infusion Dosing: Total Dose

In one embodiment, delivery of AAV particles to cells of the central nervous system, for example, a brain region, comprises prolonged infusion of at least one dose, or two or more doses. The interval between doses may be at least one hour, or between 1 hour and 120 hours. In one embodiment, the total dose of viral genomes delivered to cells of the central nervous system, for example, a brain region, defined by the equation [Total Dose VG=VG/mL*mL*# of doses] wherein VG is viral genomes and VG/mL is viral genome concentration. In accordance with the present disclosure, the total dose may be between about 1×10⁶ VG and about 1×10⁶ VG.

### Infusion Compositions

In some embodiments, a composition comprising AAV particles delivered to cells of the central nervous system, for example, a brain region, may have a certain range of concentrations, pH, baricity (i.e. density of solution), osmolarity, temperature, and other physiochemical and biochemical properties that benefit the delivery of AAV particles to cells of the central nervous system, for example, a brain region.

In one embodiment, delivery of AAV particles to cells of the central nervous system, for example, a brain region, may comprise a total dose between about 1×10⁶ VG and about 1×10¹ VG. In some embodiments, delivery may comprise a total dose of about 1×10⁶, 2×10⁶, 3×10⁶, 4×10⁶, 5×10⁶, 6×10⁶, 7×10⁶, 8×10⁶, 9×106, 1×107, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 1.9×10⁹, 2×10⁹, 3×10⁹, 4×10⁹, 5×10⁹, 6×10⁹, 7×10⁹, 8×10⁹, 9×10⁹ 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.5×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶VG.

### Pressure

In one embodiment, delivery of AAV particles to cells of the central nervous system, for example, a brain region, may comprise a rate of prolonged infusion wherein the rate of prolonged infusion exceeds the rate of CSF absorption. In some embodiments, CSF pressure may increase wherein the rate of delivery is greater than the rate of clearance. In one embodiment, increased CSF pressure may increase delivery of AAV particles to cells of the central nervous system, for example, a brain region. In one embodiment, delivery of AAV to cells of the central nervous system, for example, a brain region, may comprise an increase in sustained CSF pressure between about 1% and about 25%. In some embodiments, delivery may comprise an increase in sustained CSF pressure of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%.

Although the descriptions of pharmaceutical compositions, e.g., AAV comprising a payload to be delivered, provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In some embodiments, compositions are administered to humans, human patients or subjects. For the purposes of the present disclosure, the phrase “active ingredient” generally refers either to the viral particle carrying the payload or to the payload delivered by the viral particle as described herein.

Formulations of the AAV pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered.

### Prolonged Infusion Composition Concentration

In one embodiment, delivery of AAV particles to cells of the central nervous system, for example, a brain region, may comprise a composition concentration between about 1×10⁶ VG/mL and about 1×10¹⁶ VG/mL. In some embodiments, delivery may comprise a composition concentration of about 1×10⁶, 2×10⁶ 3×10⁶, 4×10⁶, 5×10⁶, 6×10⁶, 7×10⁶ 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10⁶¹ VG/mL. In one embodiment, delivery comprises a composition concentration of 1×10¹³ VG/mL. In one embodiment, delivery consists of a composition concentration of 1×10¹³ VG/mL. In one embodiment, delivery comprises a composition concentration of 3×10¹² VG/mL. In one embodiment, delivery consists of a composition concentration of 3×10¹² VG/mL.

### Composition pH

In one embodiment, delivery of AAV to cells of the central nervous system, for example, a brain region, comprises a buffered composition of between pH 4.5 and 8.0. In some embodiments, delivery may comprise a buffered composition of about pH 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.95, 5.1, 5.25, 5.3 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0. In one embodiment, delivery comprises a buffered composition of pH 7.4, which is considered physiological pH. In one embodiment, delivery comprises a buffered composition of pH 7.0. In one embodiment, buffer strength, or ability to hold pH, is relatively very low, allowing the infused composition to quickly adjust to the prevailing physiological pH of the CSF (˜pH 7.4).

### Composition Baricity

It is known in the art that CSF comprises a baricity, or density of solution, of approximately 1 g/mL at 37° C. In one embodiment, delivery of AAV particles to cells of the central nervous system, for example, a brain region, comprises an isobaric composition wherein the baricity of the composition at 37° C. is approximately 1 g/mL. In one embodiment, delivery comprises a hypobaric composition wherein the baricity of the composition at 37° C. is less than 1 g/mL. In one embodiment, delivery comprises a hyperbaric composition wherein the baricity of the composition at 37° C. is greater than 1 g/mL. In one embodiment, delivery comprises a hyperbaric composition wherein the baricity of the composition at 37° C. is increased by addition of approximately 5% to 8% dextrose. In one embodiment, delivery comprises a hyperbaric composition wherein the baricity of the composition at 37° C. is increased by addition of 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, or 8.0% dextrose.

### Composition Temperature

In one embodiment, delivery of AAV particles to cells of the central nervous system, for example, a brain region, comprises a composition wherein the temperature of the composition is 37° C. In one embodiment, delivery comprises a composition wherein the temperature of the composition is between approximately 20° C. and 26° C. In one embodiment, delivery comprises a composition wherein the temperature of the composition is approximately 20.0° C., 20.1° C., 20.2° C., 20.3° C., 20.4° C., 20.5° C., 20.6° C., 20.7° C., 20.8° C., 20.9° C., 21° C. 21.1° C., 21.2° C., 21.3° C., 21.4° C., 21.5° C., 21.6° C., 21.7° C., 21.8° C., 21.9° C., 22.0° C., 22.1° C., 22.2° C., 22.3° C., 22.4° C., 22.5° C., 22.6° C., 22.7° C., 22.8° C., 22.9° C., 23.0° C., 23.1° C., 23.2° C., 23.3° C., 23.4° C., 23.5° C., 23.6° C., 23.7° C., 23.8° C., 23.9° C., 24.0° C., 24.1° C., 24.2° C., 24.3° C., 24.4° C., 24.5° C., 24.6° C., 24.7° C., 24.8° C., 24.9° C., 25.0° C., 25.1° C., 25.2° C., 25.3° C., 25.4° C., 25.5° C., 25.6° C., 25.7° C., 25.8° C., 25.9° C., or 26.0° C.

### Drug Physiochemical & Biochemical Properties

In one embodiment, delivery of parvovirus e.g., AAV particles to cells of the central nervous system, for example, a brain region, comprises a composition wherein the AAV capsid is hydrophilic. In one embodiment, delivery comprises a composition wherein the AAV capsid is lipophilic.

In one embodiment, delivery of AAV particles to cells of the central nervous system, for example, a brain region, comprises a composition wherein the AAV capsid targets a specific receptor. In one embodiment, delivery of AAV particles to cells of the central nervous system, for example, a brain region, comprises a composition wherein the AAV capsid further comprises a specific ligand.

In one embodiment, delivery of AAV particles to cells of the central nervous system, for example, a brain region, comprises a composition wherein the AAV further comprises a self-complementary (SC) genome. In one embodiment, delivery comprises a composition wherein the AAV further comprises a single stranded (SS) genome.

In one embodiment, a self-complementary (SC) vector may be used to yield higher expression than the corresponding single stranded vector.

In one embodiment, delivery of AAV particles to cells of the central nervous system, for example, a brain region, comprises a composition wherein the AAV genome further comprises a cell specific promoter region. In one embodiment, delivery comprises a composition wherein the AAV genome further comprises a ubiquitous promoter region.

### Spatial Orientation

### Body Angle

In one embodiment, delivery of AAV particles to cells of the central nervous system, for example, a brain region, comprises administration to a horizontal subject. In one embodiment, delivery comprises administration to a vertical subject. In one embodiment, delivery comprises administration to a subject at an angle between approximately horizontal 0° to about vertical 90°. In one embodiment, delivery comprises administration to a subject at an angle of 0°, 1°, 2°, 3°, 4°, 5, 6°, 7°, 8°, 9°, 10°, 11°, 12°, 13°, 14°, 15°, 16°, 17°, 18°, 19°, 20°, 21°, 22°, 23°, 24°, 25°, 26°, 27°, 28°, 29°, 30°, 31°, 32°, 33°, 34°, 35°, 36°, 37°, 38°, 39°, 40°, 41°, 42°, 43°, 44°, 45°, 46°, 47°, 48°, 49°, 50°, 51°, 52°, 53°, 54°, 55°, 56°, 57°, 58°, 59°, 60°, 61°, 62°, 63°, 64°, 65°, 66° 67°, 68°, 69°, 70°, 71°, 72°, 73°, 74°, 75°, 76°, 77°, 78°, 79°, 80°, 81°, 82°, 83°, 84°, 85°, 86°, 87°, 88°, 89°, 90°.

### Change in the Orientation, Slope of Subject Body Position Over Time

In one embodiment, delivery of AAV particles to cells of the central nervous system, for example, a brain region, comprises administration to a subject wherein the angle of the subject changes over time from horizontal to vertical head up or vertical head down. In one embodiment, delivery comprises administration to a subject wherein the angle of the subject changes over time from vertical to horizontal.

In one embodiment, delivery comprises administration to a subject wherein the angle of the subject changes over time in two planes from vertical to horizontal as well as rotation around the long axis of the body. In combination, any % angle of the body can be realized between horizontal to vertical and rotationally left or right.

### Delivery Devices

In some embodiments, delivery of AAV particles to cells of the central nervous system, for example, a brain region, comprises a prolonged infusion pump or device. In some embodiments, the device may be a pump or comprise a catheter for administration of compositions of the disclosure across the blood brain barrier. Such devices include but are not limited to a pressurized olfactory delivery device, iontophoresis devices, multi-layered microfluidic devices, and the like. Such devices may be portable or stationary. They may be implantable or externally tethered to the body or combinations thereof.

Devices for administration may be employed for delivery of AAV particles to cells of the central nervous system, for example, a brain region, according to the present disclosure according to single, multi- or split-dosing regimens taught herein.

Method and devices known in the art for multi-administration to cells, organs and tissues are contemplated for use in conjunction with the methods and compositions disclosed herein as embodiments of the present disclosure. These include, for example, those methods and devices having multiple needles, hybrid devices employing for example lumens or catheters as well as devices utilizing heat, electric current or radiation driven mechanisms.

In one embodiment, the AAV particles may be delivered using an infusion port described herein and/or one that is known in the art.

In one embodiment, the AAV particles may be delivered using an infusion pump and/or an infusion port. The infusion pump and/or the infusion port may be one described herein or one known in the art such as, but not limited to, SYNCHROMED® II by Medtronic. The infusion pump may be programmed at a fixed rate or a variable rate for controlled delivery. The stability of the AAV particles and formulations thereof as well as the leachable materials should be evaluated prior to use.

In one embodiment, the devices described herein to deliver to a subject the above-described AAV particles may also include a tip protection device (e.g., for catheters and/or stereotactic fixtures of microcatheters). Non-limiting examples of protection devices are described in US Patent Publication No. US20140371711 and International Patent Publication No. WO2014204954, the contents of each of which are herein incorporated by reference in their entireties. The tip protection device may include an elongate body having a central lumen extending longitudinally therethrough, the lumen being sized and configured to slidably receive a catheter, and a locking mechanism configured to selectively maintain the elongate body in a fixed longitudinal position relative to a catheter inserted through the central lumen.

In one embodiment, the AAV particles may be delivered to a subject using a convection-enhanced delivery device. Non-limiting examples of targeted delivery of drugs using convection are described in US Patent Publication Nos. US20100217228, US20130035574 and US20130035660 and International Patent Publication No. WO2013019830 and WO2008144585, the contents of each of which are herein incorporated by reference in their entireties. The convection-enhanced delivery device may be a microfluidic catheter device that may be suitable for targeted delivery of drugs via convection, including devices capable of multi-directional drug delivery, devices that control fluid pressure and velocity using the venturi effect, and devices that include conformable balloons. As a non-limiting example, the convention-enhanced delivery device uses the venturi effect for targeted delivery of drugs as described in US Patent Publication No. US20130035574, the contents of which are herein incorporation by reference in its entirety. As another non-limiting example, the convention-enhanced delivery device uses the conformable balloons for targeted delivery of drugs as described in US Patent Publication No. US20130035660, the contents of which are herein incorporation by reference in its entirety. As another non-limiting example, the convection enhanced delivery device may be a CED catheter from Medgenesis Therapeutix such as those described in International Patent Publication No. WO2008144585 and US Patent No. US20100217228, the contents of each of which are herein incorporated by reference in their entireties. As another non-limiting example, the AAV particles may be in a liposomal composition for convection enhanced delivery such as the liposomal compositions from Medgenesis Therapeutix described in International Patent Publication No. WO2010057317 and US Patent No. US20110274625, the contents of each of which are herein incorporated by reference in their entireties, which may comprise a molar ratio of DSPC:DSPG:CHOL of 7:2:1.

In one embodiment, the catheter may be a neuromodulation catheter. Non-limiting examples of neuromodulation catheters include those taught in US Patent Application No. US20150209104 and International Publication Nos. WO2015143372, WO2015113027, WO2014189794 and WO2014150989, the contents of each of which are herein incorporated by reference in their entireties.

In one embodiment, the AAV particles may be delivered using an injection device which has a basic form of a stiff tube with holes of a selectable size at selectable places along the tube. This is a device which may be customized depending on the subject or the fluid being delivered. As a non-limiting example, the injection device which comprises a stiff tube with holes of a selectable size and location may be any of the devices described in U.S. Pat. Nos. 6,464,662, 6,572,579 and International Patent Publication No. WO2002007809, the contents of each of which are herein incorporated by reference in their entireties.

In one embodiment, the AAV particles may be delivered to a subject who is using or who has used a treatment stimulator for brain diseases. Non-limiting examples include treatment stimulators from THERATAXIS™ and the treatment stimulators described in International Patent Publication No. WO2008144232, the contents of which are herein incorporated by reference in its entirety.

In one embodiment, the AAV particles may be delivered to a defined area using a medical device which comprises a sealing system proximal to the delivery end of the device. Non-limiting examples of medical device which can deliver AAV particles to a defined area includes U.S. Pat. No. 7,998,128, US Patent Application No. US20100030102 and International Patent Publication No. WO2007133776, the contents of each of which are herein incorporated by reference in their entireties.

In one embodiment, the AAV particle may be delivered over an extended period of time using an extended delivery device. Non-limiting examples of extended delivery devices are described in International Patent Publication Nos. WO2015017609 and WO2014100157, U.S. Pat. No. 8,992,458, and US Patent Publication Nos. US20150038949, US20150133887 and US20140171902, the contents of each of which are herein incorporated by reference in their entireties. As a non-limiting example, the devices used to deliver the AAV particles are CED devices with various features for reducing or preventing backflow as in International Patent Publication No. WO2015017609 and US Patent Publication No. US20150038949, the contents of each of which are herein incorporated by reference in their entireties. As another non-limiting example, the devices used to deliver the AAV particles are CED devices which include a bullet-shaped nose proximal to a distal fluid outlet where the bullet-shaped nose forms a good seal with surrounding tissue and helps reduce or prevent backflow of infused fluid as described in U.S. Pat. No. 8,992,458, US Patent Publication Nos. US20150133887 and US20140171902 and International Patent Publication No. WO2014100157, the contents of each of which are herein incorporated by reference by their entireties. As another non-limiting example, the catheter may be made using micro-electro-mechanical systems (MEMS) technology to reduce backflow as described by Brady et al. (Journal of Neuroscience Methods 229 (2014) 76-83), the contents of which are herein incorporated by reference in its entirety.

In one embodiment, the AAV particles may be delivered using an implantable delivery device. Non-limiting examples of implantable devices are described by and sold by Codman Neuro Sciences (Le Locle, CH). The implantable device may be an implantable pump such as, but not limited to, those described in U.S. Pat. Nos. 8,747,391, 7,931,642, 7,637,897, and 6,755,814 and US Patent Publication No. US20100069891, the contents of each of which are herein incorporated by reference in their entireties. The implantable device (e.g., a fluidic system) may have the flow rate accuracy of the device optimized by the methods described in U.S. Pat. Nos. 8,740,182 and 8,240,635, and US Patent Publication No. US20120283703, the contents of each of which are herein incorporated by reference in its entirety. As a non-limiting example, the duty cycle of the valve of a system may be optimized to achieve the desired flow rate. The implantable device may have an electrokinetic actuator for adjusting, controlling or programming fine titration of fluid flow through a valve mechanism without intermixing between the electrolyte and fluid. As a non-limiting example, the electrokinetic actuator may be any of those described in U.S. Pat. No. 8,231,563 and US Patent Publication No. US20120283703, the contents of which are herein incorporated by reference in its entirety. Fluids of an implantable infusion pump may be monitored using methods known in the art and those taught in U.S. Pat. No. 7,725,272, the contents of which are herein incorporated by reference in its entirety.

In one embodiment, the delivery of the AAV particles in a subject may be determined and/or predicted using the prediction methods described in International Patent Publication No. WO2001085230, the contents of which are herein incorporated by reference in its entirety.

In one embodiment, a subject may be imaged prior to, during and/or after administration of the AAV particles. The imaging method may be a method known in the art and/or described herein. As a non-limiting example, the imaging method which may be used to classify brain tissue includes the medical image processing method described in U.S. Pat. Nos. 7,848,543, 9,101,282 and EP Application No. EP1768041, the contents of each of which are herein incorporated by reference in their entireties. As yet another non-limiting example, the physiological states and the effects of treatment of a neurological disease in a subject may be tracked using the methods described in US Patent Publication No. US20090024181, the contents of which are herein incorporated by reference in its entirety.

In one embodiment, a device may be used to deliver the AAV particles where the device creates one or more channels, tunnels or grooves in tissue in order to increase hydraulic conductivity. These channels, tunnels or grooves will allow the AAV particles to flow and produce a predictable infusion pattern. Non-limiting examples of this device are described in U.S. Pat. No. 8,083,720, US Patent Application No. US20110106009, and International Publication No. WO2009151521, the contents of each of which are herein incorporated by reference in its entirety.

In one embodiment, the flow of a composition comprising the AAV particles may be controlled using acoustic waveform outside the target area. Non-limiting examples of devices, methods and controls for using sonic guidance to control molecules is described in US Patent Application No. US20120215157, U.S. Pat. No. 8,545,405, International Patent Publication Nos. WO2010096495 and WO2010080701, the contents of each of which are herein incorporated by reference in their entireties.

In one embodiment, the flow of a composition comprising the AAV particles may be modeled prior to administration using the methods and apparatus described in U.S. Pat. Nos. 6,549,803 and 8,406,850 and US Patent Application No. US20080292160, the content of each of which is incorporated by reference in their entireties. As a non-limiting example, the physiological parameters defining edema induced upon infusion of fluid from an intraparenchymally placed catheter may be estimated using the methods described in U.S. Pat. No. 8,406,850 and US Patent Application No. US20080292160, the contents of which is herein incorporated by reference in its entirety.

In one embodiment, a surgical alignment device may be used to deliver the AAV particles to a subject. The surgical alignment device may be a device described herein and/or is known in the art. As a non-limiting example, the surgical alignment device may be controlled remotely (i.e., robotic) such as the alignment devices described in U.S. Pat. Nos. 7,366,561 and 8,083,753, the contents of each of which is incorporated by reference in their entireties.

In one embodiment, an intraparenchymal (IPA) catheter from Alcyone may be used to deliver the AAV particles described herein.

In another embodiment, an intraparenchymal catheter from Atanse may be used to deliver the AAV particles described herein.

In one embodiment, the distribution of the AAV particles described herein may be evaluated using imaging technology from Therataxis and/or Brain Lab.

### V. Administration and Dosing

### Administration

In one embodiment, an AAV particle comprising one or more capsid protein serotypes and/or sequences of Table 1 for use in delivery of payloads to a central nervous system region, for example, a brain region, via administration to the CSF may be administered to a subject (e.g., to the CNS of a subject) in a therapeutically effective amount to reduce the symptoms of neurological disease of a subject (e.g., determined using a known evaluation method).

In various non-limiting examples, AAV particles comprising one or more capsid protein serotypes and/or sequences of Table 1 can be used for delivery of payloads to a brain region, via administration to the CSF where the brain region is the frontal cortex, occipital cortex, caudate nucleus, putamen, thalamus, hippocampus, cingulate gyrus, hypothalamus, pons, medulla, cerebellar Purkinje layer, or cerebellar granular layer for treatment, prophylaxis, palliation or amelioration of neurological diseases and/or disorders.

In some embodiments, compositions may be administered in a way which allows them to bypass the blood brain barrier, vascular barrier, or other epithelial barrier and directly access cerebrospinal fluid (CSF).

In one embodiment, the AAV particles comprising one or more capsid protein serotypes and/or sequences of Table 1 may be delivered by injection into the CSF pathway. Non-limiting examples of delivery to the CSF pathway include cisterna magna (CM), intrathecal (IT), and intracerebroventricular (ICV) administration.

In some embodiments, the AAV particles comprising one or more capsid protein serotypes and sequences of Table 1 described herein may be administered by intrathecal (IT) injection. As a non-limiting example, the AAV particles described herein may be administered by intrathecal injection.

In one embodiment, the AAV particle may be administered to the cisterna magna (CM) in a therapeutically effective amount to transduce various brain regions of the CNS. Non-limiting examples of various brain regions include frontal cortex, occipital cortex, caudate nucleus, putamen, thalamus, hippocampus, cingulate gyrus, hypothalamus, pons, medulla, cerebellar Purkinje layer, and cerebellar granular layer. As a non-limiting example, the AAV particle may be administered intrathecally.

In one embodiment, the AAV particle may be administered using intrathecal infusion in a therapeutically effective amount to transduce various brain regions of the CNS including frontal cortex, occipital cortex, caudate nucleus, putamen, thalamus, hippocampus, cingulate gyrus, hypothalamus, pons, medulla, cerebellar Purkinje layer, and cerebellar granular layer.

In some embodiments, the AAV particles comprising one or more capsid protein serotypes and/or sequences of Table 1 described herein may be administered via a single dose intrathecal injection. As a non-limiting example, the single dose intrathecal injection may be a one-time treatment. In some embodiments, the AAV particles comprising one or more capsid protein serotypes and/or sequences of Table 1 described herein may be administered via intrathecal injection to various brain regions of the CNS including frontal cortex, occipital cortex, caudate nucleus, putamen, thalamus, hippocampus, cingulate gyrus, hypothalamus, pons, medulla, cerebellar Purkinje layer, and cerebellar granular layer.

In some embodiments, the AAV particles comprising one or more capsid protein serotypes and/or sequences of Table 1 described herein may be administered via a single dose intrathecal injection to various brain regions of the CNS including frontal cortex, occipital cortex, caudate nucleus, putamen, thalamus, hippocampus, cingulate gyms, hypothalamus, pons, medulla, cerebellar Purkinje layer, and cerebellar granular layer. As a non-limiting example, the single dose intrathecal injection may be a one-time treatment.

In one embodiment, the AAV particle described herein is administered via intrathecal (IT) infusion at C1. The infusion may be for 1, 2, 3, 4, 6, 7, 8, 9, 10, 11, 12, 13, 14, 15 or more than 15 hours.

In some embodiments, the AAV particles comprising one or more capsid protein serotypes and/or sequences of Table 1 described herein may be administered by intracerebroventricular (ICV) injection. As a non-limiting example, the AAV particles described herein may be administered by intracerebroventricular (ICV) injection.

In one embodiment, the AAV particle may be administered by intracerebroventricular (ICV) injection in a therapeutically effective amount to transduce various brain regions of the CNS. Non-limiting examples of various brain regions include frontal cortex, occipital cortex, caudate nucleus, putamen, thalamus, hippocampus, cingulate gyrus, hypothalamus, pons, medulla, cerebellar Purkinje layer, and cerebellar granular layer. As a non-limiting example, the AAV particle may be administered by intracerebroventricular (ICV) injection.

In one embodiment, a subject may be administered the AAV particles comprising one or more capsid protein serotypes and/or sequences of Table 1 described herein using sustained delivery over a period of minutes, hours or days. The infusion rate may be changed depending on the subject, distribution, formulation or another delivery parameter.

In one embodiment, the AAV particles comprising one or more capsid protein serotypes and/or sequences of Table 1 described herein may be administered to a subject by intracranial delivery (See, e.g., U.S. Pat. No. 8,119,611; the content of which is incorporated herein by reference in its entirety).

In one embodiment, the AAV particle may be administered to the CNS, for example to a brain region, by administration to CSF in a therapeutically effective amount to improve function and/or survival for a subject with a neurological disease. The AAV particle may be administered in a “therapeutically effective” amount, i.e., an amount that is sufficient to alleviate and/or prevent at least one symptom associated with the disease, or provide improvement in the condition of the subject.

In one embodiment, the catheter may be located at more than one site in the spine for multi-site delivery. The AAV particle may be delivered in a continuous and/or bolus infusion. Each site of delivery may be a different dosing regimen or the same dosing regimen may be used for each site of delivery. As a non-limiting example, the sites of delivery may be in the cervical and the lumbar region. As another non-limiting example, the sites of delivery may be in the cervical region. As another non-limiting example, the sites of delivery may be in the lumbar region.

In one embodiment, a subject may be analyzed for spinal anatomy and pathology prior to delivery of the AAV particle described herein. As a non-limiting example, a subject with scoliosis may have a different dosing regimen and/or catheter location compared to a subject without scoliosis.

In one embodiment, the orientation of the spine of the subject during delivery of the AAV particle may be vertical to the ground.

In another embodiment, the orientation of the spine of the subject during delivery of the AAV particle may be horizontal to the ground.

In one embodiment, the spine of the subject may be at an angle as compared to the ground during the delivery of the AAV particle. The angle of the spine of the subject as compared to the ground may be at least 10, 20, 30, 40, 50, 60, 70, 80, 90, 10, 110, 120, 130, 140, 150 or 180 degrees.

In one embodiment, the delivery method and duration is chosen to provide broad transduction in the spinal cord. As a non-limiting example, intrathecal delivery is used to provide broad transduction along the rostral-caudal length of the spinal cord. As another non-limiting example, multi-site infusions provide a more uniform transduction along the rostral-caudal length of the spinal cord. As yet another non-limiting example, prolonged infusions provide a more uniform transduction along the rostral-caudal length of the spinal cord.

In some embodiments, pharmaceutical compositions, AAV particles described herein are formulated in depots for extended release.

### Delivery, Dose, and Regimen

In one aspect, the present disclosure provides methods of administering AAV particles comprising one or more capsid protein serotypes and/or sequences of Table 1 described herein to a subject in need thereof.

In one embodiment, the AAV particle may be delivered in a multi-dose regimen. The multi-dose regimen may be 2, 3, 4, 5, 6, 7, 8, 9, 10 or more than 10 doses.

In one embodiment, the AAV particle may be delivered to a subject via a multi-site route of administration. A subject may be administered the AAV particle at 2, 3, 4, 5 or more than 5 sites.

The desired dosage of the AAV particles described herein may be delivered only once, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of “single unit dose” or total daily dose into two or more doses, e.g., two or more administrations of the “single unit dose”. As used herein, a “single unit dose” is a dose of any therapeutic administered in one dose/at one time/single route/single point of contact, i.e., single administration event.

The desired dosage of the AAV particles described herein may be administered as a “pulse dose” or as a “continuous flow”. As used herein, a “pulse dose” is a series of single unit doses of any therapeutic administered with a set frequency over a period of time. As used herein, a “continuous flow” is a dose of therapeutic administered continuously for a period of time in a single route/single point of contact, i.e., continuous administration event. A total daily dose, an amount given or prescribed in 24 hour period, may be administered by any of these methods, or as a combination of these methods, or by any other methods suitable for a pharmaceutical administration.

In one embodiment, delivery of the AAV particles described herein to a subject provides regulating activity of a target gene in a subject. The regulating activity may be an increase in the production of the target protein in a subject or the decrease of the production of target protein in a subject. The regulating activity can be for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 20 months, 21 months, 22 months, 23 months, 2 years, 3 years, 4 years, 5 years, 6 years, 7 years, 8 years, 9 years, 10 years or more than 10 years.

In some embodiments, the AAV particle described herein may be administered to a subject using a single dose, one-time treatment. The dose of the one-time treatment may be administered by any methods known in the art and/or described herein. As used herein, a “one-time treatment” refers to a composition which is only administered one time. If needed, a booster dose may be administered to the subject to ensure the appropriate efficacy is reached. A booster may be administered 1 month, 2 months, 3 months, 4 months, 5 months, 6 months, 7 months, 8 months, 9 months, 10 months, 11 months, 12 months, 1 year, 13 months, 14 months, 15 months, 16 months, 17 months, 18 months, 19 months, 20 months, 21 months, 22 months, 23 months, 24 months, 2 years, 3 years, 4 years, 5 years, 6 years, 7 years, 8 years, 9 years, 10 years, or more than 10 years after the one-time treatment.

### Measurement of Expression

Expression of payloads from viral genomes may be determined using various methods known in the art such as, but not limited to immunochemistry (e.g., IHC), in situ hybridization (ISH), enzyme-linked immunosorbent assay (ELISA), affinity ELISA, ELISPOT, flow cytometry, immunocytology, surface plasmon resonance analysis, kinetic exclusion assay, liquid chromatography-mass spectrometry (LCMS), high-performance liquid chromatography (HPLC). BCA assay, immunoelectrophoresis. Western blot, SDS-PAGE, protein immunoprecipitation, and/or PCR.

### VI. AAV Production

The present disclosure provides methods for the generation of parvoviral particles, e.g. AAV particles, comprising one or more capsid protein serotypes and/or sequences of Table 1 by viral genome replication in a viral replication cell.

In accordance with the present disclosure, the viral genome comprising a payload region will be incorporated into the AAV particle comprising one or more capsid protein serotypes and/or sequences of Table 1 and produced in a viral replication cell. Methods of making AAV particles are well known in the art and are described in e.g., United States Patent Nos. U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948; or International Publication Nos. WO1996039530, WO1998010088, WO1999014354, WO1999015685, WO1999047691, WO2000055342, WO2000075353 and WO2001023597; Methods In Molecular Biology, ed. Richard, Humana Press, NJ (1995); O'Reilly et al., Baculovirus Expression Vectors, A Laboratory Manual, Oxford Univ. Press (1994); Samulski et al., J Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir., 219:37-44 (1996); Zhao et al., J. Vir. 272:382-93 (2000); the contents of each of which are herein incorporated by reference in their entirety. In one embodiment, the AAV particles are made using the methods described in WO2015191508, the contents of which are herein incorporated by reference in their entirety.

Viral replication cells commonly used for production of recombinant AAV viral vectors include but are not limited to HEK293 cells, COS cells, HeLa cells, KB cells, and other mammalian cell lines as described in U.S. Pat. Nos. U.S. Pat. Nos. 6,156,303, 5,387,484, 5,741,683, 5,691,176, and 5,688,676; U.S. patent publication No. 2002/0081721, and International Patent Publication Nos. WO 00/47757, WO 00/24916, and WO 96/17947, the contents of each of which are herein incorporated by reference in their entireties.

In some embodiments, the present disclosure provides a method for producing an AAV particle comprising one or more capsid protein serotypes and/or sequences of Table 1 wherein the particle has enhanced (increased, improved) transduction efficiency comprising the steps of 1) co-transfecting competent bacterial cells with a bacmid vector and either a viral construct vector and/or AAV payload construct vector, 2) isolating the resultant viral construct expression vector and AAV payload construct expression vector and separately transfecting viral replication cells, 3) isolating and purifying resultant payload and viral construct particles comprising viral construct expression vector or AAV payload construct expression vector, 4) co-infecting a viral replication cell with both the AAV payload and viral construct particles comprising viral construct expression vector or AAV payload construct expression vector, and 5) harvesting and purifying the AAV particle comprising a viral genome.

In some embodiments, the present disclosure provides a method for producing an AAV particle comprising one or more capsid proteins described herein, wherein the method comprises the steps of 1) simultaneously co-transfecting mammalian cells, such as, but not limited to HEK293 cells, with a payload region, a construct expressing rep and cap genes and a helper construct, 2) harvesting and purifying the AAV particle comprising a viral genome.

In one embodiment, the viral construct vector(s) used for AAV production may contain a nucleotide sequence encoding the AAV capsid proteins where the initiation codon of the AAV VP capsid protein is a non-ATG, i.e., a suboptimal initiation codon, allowing the expression of a modified ratio of the viral capsid proteins in the production system, to provide improved infectivity of the host cell. In a non-limiting example, a viral construct vector may contain a nucleic acid construct comprising a nucleotide sequence encoding AAV VP1, VP2, and VP3 capsid proteins, wherein the initiation codon for translation of the AAV VP1 capsid protein is CTG, TTG, or GTG, as described in U.S. Pat. No. 8,163,543, the contents of which are herein incorporated by reference in its entirety.

In one embodiment, the viral construct vector(s) used for AAV production may contain a nucleotide sequence encoding the AAV rep proteins where the initiation codon of the AAV rep protein or proteins is a non-ATG. In one embodiment, a single coding sequence is used for the Rep78 and Rep52 proteins, wherein initiation codon for translation of the Rep78 protein is a suboptimal initiation codon, selected from the group consisting of ACG, TTG, CTG and GTG, that effects partial exon skipping upon expression in insect cells, as described in U.S. Pat. No. 8,512,981, the contents of which is herein incorporated by reference in its entirety, for example to promote less abundant expression of Rep78 as compared to Rep52, which may be advantageous in that it promotes high vector yields.

In some embodiments, the viral genome of the AAV particle comprising one or more capsid protein serotypes and/or sequences of Table 1 optionally encodes a selectable marker. The selectable marker may comprise a cell-surface marker, such as any protein expressed on the surface of the cell including, but not limited to receptors, CD markers, lectins, integrins, or truncated versions thereof.

In some embodiments, selectable marker reporter genes used are as described in International application No. WO 96/23810; Heim et al., Current Biology 2:178-182 (1996); Heim et al., Proc. Natl. Acad. Sci. USA (1995); or Heim et al., Science 373:663-664 (1995); WO 96/30540, the contents of each of which are incorporated herein by reference in their entireties).

In certain embodiments, provided herein is a method for producing an AAV particle comprising one or more capsid protein serotypes and/or sequences of Table 1 whereby the particle is produced by insect cells, for example, by using an Sf9/baculovirus insect cell system.

In one embodiment, the present disclosure provides a method of making a population of parvovirus (e.g., AAV) particles comprising one or more capsid proteins described herein, wherein the method comprises: (a) culturing insect cells to produce a population of parvovirus (e.g., AAV) particles; and (b) harvesting the population of parvovirus particles produced by the insect cells. For example, in one embodiment, the present disclosure provides a method of making a population of parvovirus (e.g., AAV) particles comprising one or more capsid proteins described herein, wherein the method comprises: (a) culturing insect cells comprising one or more baculovirus expression vectors, or BEVs, to produce a population of parvovirus (e.g., AAV) particles; and (b) harvesting the population of parvovirus particles produced by the insect cells.

A BEV is a baculovirus plasmid or bacmid having a viral construct for expression of non-structural and structural proteins and/or a payload construct as described herein. In this context, “non-structural proteins” refer to proteins involved in parvovirus (e.g., AAV) replication, including site specific endonuclease and helicase activity, DNA replication and activation of promoters during transcription, or proteins that are required for assembly of the capsid of a parvovirus particle. Also, in this context, “structural proteins” refer to capsid proteins, such as VP1. VP2 and VP3 capsid proteins described herein, of a parvovirus, e.g., AAV particle.

In the context of AAV, the rep gene encodes the non-structural Rep proteins of Rep78, Rep68, Rep52 and Rep40, which in the plasmid(s) or bacmid(s) can be expressed via single or multiple, separate, coding sequences and the ORF2 of the cap gene encodes the non-structural Assembly-Activating Protein (AAP). Methods for introducing such constructs into a baculovirus plasmid or bacmid are well known in the art, which can include use of a transposon donor/acceptor system.

In some embodiments, the present disclosure provides a method for producing a population of parvovirus (e.g., AAV) particles comprising one or more capsid proteins described herein, wherein the method comprises: (a) culturing insect cells; (b) infecting the insect cells with a first BIIC and a second BIIC, wherein the first BIIC includes a baculovirus expression vector including a nucleotide sequence that produces a parvovirus (e.g., AAV) viral genome described herein, and wherein the second BIIC includes a baculovirus expression vector including a nucleotide sequence that produces parvovirus (e.g., AAV) non-structural and structural proteins necessary for parvovirus (e.g., AAV) particle formation in the insect cells; and (c) harvesting the parvovirus particles produced by the insect cells following the infection step A BIIC is a “baculovirus infected insect cell” and refers to an insect cell that has been infected with a BEV.

Growing conditions for insect cells in culture, and production of heterologous products in insect cells in culture are well-known in the art, see U.S. Pat. No. 6,204,059, the contents of which are herein incorporated by reference in their entirety.

Any insect cell which allows for replication of parvovirus and which can be maintained in culture can be used in accordance with the present disclosure. Cell lines can be used from Spodoptera frugiperda, including, but not limited to the pupal ovarian Sf9 or Sf21 cell lines, drosophila cell lines, or mosquito cell lines, such as, Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, METHODS IN MOLECULAR BIOLOGY, ed. Richard, Humana Press, N J (1995); O'Reilly et al., BACULOVIRUS EXPRESSION VECTORS, A LABORATORY MANUAL. Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J Vir. 66:6922-30 (1992); Kimbaucr et al., Vir. 219:37-44 (1996); Zhao et al., Vir. 272:382-93 (2000); and Samulski et al., U.S. Pat. No. 6,204,059, the contents of each of which are herein incorporated by reference in their entirety.

Baculovirus expression vectors for producing parvovirus (e.g., AAV) particles in insect cells, including but not limited to Spodoptera frugiperda (Sf9) cells, provide high titers of parvovirus (e.g., AAV) particle product. Recombinant baculovirus encoding the viral construct expression vector and payload construct expression vector initiates a productive infection of viral replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al. J Virol. 2006 February; 80(4):1874-85, the contents of which are herein incorporated by reference in their entirety.

In one embodiment, a genetically stable baculovirus can be used to produce the source of one or more of the components for producing parvovirus (e.g., AAV) particles in invertebrate cells. In one embodiment, defective baculovirus expression vectors can be maintained episomally in insect cells. In such an embodiment, the bacmid vector is engineered with replication control elements, including but not limited to promoters, enhancers, and/or cell-cycle regulated replication elements.

In some embodiments, baculoviruses can be engineered with a (non-) selectable marker for recombination into the chitinase/cathepsin locus. The chia/v-cath locus is non-essential for propagating baculovirus in tissue culture, and the V-cath (EC 3.4.22.50) is a cysteine endoprotease that is most active on Arg-Arg dipeptide containing substrates. The Arg-Arg dipeptide is present in densovirus and parvovirus capsid structural proteins but infrequently occurs in dependovirus VP1.

In some embodiments, stable viral replication cells permissive for baculovirus infection are engineered with at least one stable integrated copy of any of the elements necessary for AAV replication and parvovirus particle production including, but not limited to, i) the entire AAV genome, ii) Rep genes and polynucleotide sequences that express capsid protein coding sequences described herein (either as a single or separate open reading frames), iii) Rep genes, iv) polynucleotide sequences that express capsid protein coding sequences (either as single or separate open reading frames), v) polynucleotides that express each Rep protein coding sequence as a separate transcription cassette, vi) polynucleotides that express each capsid VP protein coding sequence as a separate transcription/expression cassette, vii) the AAP (assembly activation protein), and/or viii) at least one of the baculovirus helper genes with native or non-native promoters.

In some embodiments, large-scale viral production methods can include the use of suspension cell cultures. Suspension cell culture allows for significantly increased numbers of cells. Typically, the number of adherent cells that can be grown on about 10-50 cm² of surface area can be grown in about 1 cm volume in suspension.

Transfection of replication cells in large-scale culture formats can be carried out according to any methods known in the art. For large-scale adherent cell cultures, transfection methods can include, but are not limited to the use of inorganic compounds (e.g. calcium phosphate,) organic compounds [e.g. polyethyleneimine (PEI)] or the use of non-chemical methods (e.g. electroporation). With cells grown in suspension, transfection methods can include, but are not limited to the use of calcium phosphate and the use of PEI. In some cases, transfection of large scale suspension cultures can be carried out according to the section entitled “Transfection Procedure” described in Feng, L. et al., 2008. Biotechnol Appl Biochem. 50:121-32, the contents of which are herein incorporated by reference in their entirety. According to such embodiments, PEI-DNA complexes can be formed for introduction of plasmids to be transfected. In some cases, cells being transfected with PEI-DNA complexes can be ‘shocked’ prior to transfection. This includes lowering cell culture temperatures to 4° C. for a period of about 1 hour. In some cases, cell cultures can be shocked for a period of from about 10 minutes to about 5 hours. In some cases, cell cultures can be shocked at a temperature of from about 0° C. to about 20° C.

In some cases, transfections can include one or more vectors for expression of an RNA effector molecule to reduce expression of nucleic acids from one or more payload construct. Such methods can enhance the production of parvovirus particles by reducing cellular resources wasted on expressing payload constructs. In some cases, such methods can be carried according to those taught in US Publication No. US2014/0099666, the contents of which are herein incorporated by reference in their entirety.

Cells described herein, including, but not limited to viral production cells, can be subjected to cell lysis according to any methods known in the art. Cell lysis can be carried out to obtain one or more agents (e.g. parvovirus particles) present within any cells described herein. In some embodiments, cell lysis can be carried out according to any of the methods listed in U.S. Pat. Nos. 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935, 7,968,333, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety. Cell lysis methods can be chemical or mechanical. Chemical cell lysis typically includes contacting one or more cells with one or more lysis agents. Mechanical lysis typically includes subjecting one or more cells to one or more lysis conditions and/or one or more lysis forces.

In some embodiments, chemical lysis can be used to lyse cells. As used herein, the term lysis agent refers to any agent that can aid in the disruption of a cell. In some cases, lysis agents are introduced in solutions, termed lysis solutions or lysis buffers. As used herein, the term lysis solution refers to a solution (typically aqueous) including one or more lysis agents. In addition to lysis agents, lysis solutions can include one or more buffering agents, solubilizing agents, surfactants, preservatives, cryoprotectants, enzymes, enzyme inhibitors and/or chelators. Lysis buffers are lysis solutions including one or more buffering agents. Additional components of lysis solutions can include one or more solubilizing agents. As used herein, the term solubilizing agent refers to a compound that enhances the solubility of one or more components of a solution and/or the solubility of one or more entities to which solutions are applied. In some cases, solubilizing agents enhance protein solubility. In some cases, solubilizing agents are selected based on their ability to enhance protein solubility while maintaining protein conformation and/or activity.

Exemplary lysis agents can include any of those described in U.S. Pat. Nos. 8,685,734, 7,901,921, 7,732,129, 7,223,585, 7,125,706, 8,236,495, 8,110,351, 7,419,956, 7,300,797, 6,699,706 and 6,143,567, the contents of each of which are herein incorporated by reference in their entirety. In some cases, lysis agents can be selected from lysis salts, amphoteric agents, cationic agents, ionic detergents and non-ionic detergents. Lysis salts can include, but are not limited to sodium chloride (NaCl) and potassium chloride (KCl). Further lysis salts can include any of those described in U.S. Pat. Nos. 8,614,101, 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935 and 7,968,333, the contents of each of which are herein incorporated by reference in their entirety. Concentrations of salts can be increased or decreased to obtain an effective concentration for rupture of cell membranes. Amphoteric agents, as referred to herein, are compounds capable of reacting as an acid or a base. Amphoteric agents can include, but are not limited to lysophosphatidylcholine, 3-((3-Cholamidopropyl)dimethylammonium)-1-propanesulfonate (CHAPS), ZWITERGENT® and the like. Cationic agents can include, but are not limited to cetyltrimethylammonium bromide (C(16)TAB) and Benzalkonium chloride. Lysis agents including detergents can include ionic detergents or non-ionic detergents. Detergents can function to break apart or dissolve cell structures including, but not limited to cell membranes, cell walls, lipids, carbohydrates, lipoproteins and glycoproteins. Exemplary ionic detergents include any of those taught in U.S. Pat. Nos. 7,625,570 and 6,593,123 or US Publication No. US2014/0087361, the contents of each of which are herein incorporated by reference in their entirety. Some ionic detergents can include, but are not limited to sodium dodecyl sulfate (SDS), cholate and deoxycholate. In some cases, ionic detergents can be included in lysis solutions as a solubilizing agent. Non-ionic detergents can include, but are not limited to octylglucoside, digitonin, lubrol, C12E8, TWEEN®-20, TWEEN®-80, Triton X-100 and Noniodet P40. Non-ionic detergents are typically weaker lysis agents, but can be included as solubilizing agents for solubilizing cellular and/or viral proteins. Further lysis agents can include enzymes and urea. In some cases, one or more lysis agents can be combined in a lysis solution in order to enhance one or more of cell lysis and protein solubility. In some cases, enzyme inhibitors can be included in lysis solutions in order to prevent proteolysis that can be triggered by cell membrane disruption.

In some embodiments, mechanical cell lysis is carried out. Mechanical cell lysis methods can include the use of one or more lysis conditions and/or one or more lysis forces. As used herein, the term lysis condition refers to a state or circumstance that promotes cellular disruption. Lysis conditions can include certain temperatures, pressures, osmotic purity, salinity and the like. In some cases, lysis conditions include increased or decreased temperatures. According to some embodiments, lysis conditions include changes in temperature to promote cellular disruption. Cell lysis carried out according to such embodiments can include freeze-thaw lysis. As used herein, the term freeze-thaw lysis refers to cellular lysis in which a cell solution is subjected to one or more freeze-thaw cycles. According to freeze-thaw lysis methods, cells in solution are frozen to induce a mechanical disruption of cellular membranes caused by the formation and expansion of ice crystals. Cell solutions used according to freeze-thaw lysis methods, can further include one or more lysis agents, solubilizing agents, buffering agents, cryoprotectants, surfactants, preservatives, enzymes, enzyme inhibitors and/or chelators. Once cell solutions subjected to freezing are thawed, such components can enhance the recovery of desired cellular products. In some cases, one or more cryoprotectants are included in cell solutions undergoing freeze-thaw lysis. As used herein, the term “cryoprotectant” refers to an agent used to protect one or more substances from damage due to freezing. Cryoprotectants described herein can include any of those taught in US Publication No. US2013/0323302 or U.S. Pat. No. 6,503,888, 6,180,613, 7,888,096, 7,091,030, the contents of each of which are herein incorporated by reference in their entirety. In some cases, cryoprotectants can include, but are not limited to dimethyl sulfoxide, 1,2-propanediol, 2,3-butanediol, formamide, glycerol, ethylene glycol, 1,3-propanediol and n-dimethyl formamide, polyvinylpyrrolidone, hydroxyethyl starch, agarose, dextrans, inositol, glucose, hydroxyethylstarch, lactose, sorbitol, methyl glucose, sucrose and urea. In some embodiments, freeze-thaw lysis can be carried out according to any of the methods described in U.S. Pat. No. 7,704,721, the contents of which are herein incorporated by reference in their entirety.

As used herein, the term lysis force refers to a physical activity used to disrupt a cell. Lysis forces can include, but are not limited to mechanical forces, sonic forces, gravitational forces, optical forces, electrical forces and the like. Cell lysis carried out by mechanical force is referred to herein as mechanical lysis. Mechanical forces that can be used according to mechanical lysis can include high shear fluid forces. According to such methods of mechanical lysis, a microfluidizer can be used. Microfluidizers typically include an inlet reservoirs where cell solutions can be applied. Cell solutions can then be pumped into an interaction chamber via a pump (e.g. high-pressure pump) at high speed and/or pressure to produce shear fluid forces. Resulting lysates can then be collected in one or more output reservoir. Pump speed and/or pressure can be adjusted to modulate cell lysis and enhance recovery of products (e.g. parvovirus particles). Other mechanical lysis methods can include physical disruption of cells by scraping.

Cell lysis methods can be selected based on the cell culture format of cells to be lysed. For example, with adherent cell cultures, some chemical and mechanical lysis methods can be used. Such mechanical lysis methods can include freeze-thaw lysis or scraping. In another example, chemical lysis of adherent cell cultures can be carried out through incubation with lysis solutions including surfactant, such as Triton-X-100. In some cases, cell lysates generated from adherent cell cultures can be treated with one or more nucleases to lower the viscosity of the lysates caused by liberated DNA.

Cell lysates including parvovirus (e.g., AAV) particles comprising one or more capsid protein serotypes and/or sequences of Table 1 can be subjected to clarification. Clarification refers to initial steps taken in purification of parvovirus particles from cell lysates. Clarification serves to prepare lysates for further purification by removing larger, insoluble debris. Clarification steps can include, but are not limited to centrifugation and filtration. During clarification, centrifugation can be carried out at low speeds to remove larger debris, only. Similarly, filtration can be carried out using filters with larger pore sizes so that only larger debris is removed. In some cases, tangential flow filtration can be used during clarification. Objectives of viral clarification include high throughput processing of cell lysates and optimization of ultimate viral recovery. Advantages of including a clarification step include scalability for processing of larger volumes of lysate. In some embodiments, clarification can be carried out according to any of the methods presented in U.S. Pat. Nos. 8,524,446, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498, 7,491,508, US Publication Nos, US2013/0045186, US2011/0263027, US2011/0151434, US2003/0138772, and International Publication Nos. WO2002012455, WO1996039530, WO1998010088, WO1999014354, WO1999015685, WO1999047691, WO2000055342, WO2000075353 and WO2001023597, the contents of each of which are herein incorporated by reference in their entirety.

Methods of cell lysate clarification by filtration are well understood in the art and can be carried out according to a variety of available methods including, but not limited to passive filtration and flow filtration. Filters used can include a variety of materials and pore sizes. For example, cell lysate filters can include pore sizes of from about 1 μM to about 5 μM, from about 0.5 μM to about 2 μM, from about 0.1 μM to about 1 μM, from about 0.05 μM to about 0.5 μM and from about 0.001 μM to about 0.1 μM. Exemplary pore sizes for cell lysate filters can include, but are not limited to, 2.0, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.95, 0.9, 0.85, 0.8, 0.75, 0.7, 0.65, 0.6, 0.55, 0.5, 0.45, 0.4, 0.35, 0.3.0.25, 0.2, 0.15, 0.1, 0.05, 0.22, 0.21, 0.20, 0.19, 0.18, 0.17, 0.16, 0.15, 0.14, 0.13, 0.12, 0.11, 0.1, 0.09, 0.08, 0.07, 0.06, 0.05, 0.04, 0.03, 0.02, 0.01, 0.02, 0.019, 0.018, 0.017, 0.016, 0.015, 0.014, 0.013, 0.012, 0.011, 0.01, 0.009, 0.008, 0.007, 0.006, 0.005, 0.004, 0.003, 0.002, 0.001 and 0.001 μM. In one embodiment, clarification can include filtration through a filter with 2.0 μM pore size to remove large debris, followed by passage through a filter with 0.45 μM pore size to remove intact cells.

Filter materials can be composed of a variety of materials. Such materials can include, but are not limited to polymeric materials and metal materials (e.g. sintered metal and pored aluminum). Exemplary materials can include, but are not limited to nylon, cellulose materials (e.g. cellulose acetate), polyvinylidene fluoride (PVDF), polyethersulfone, polyamide, polysulfone, polypropylene and polyethylene terephthalate. In some cases, filters useful for clarification of cell lysates can include, but are not limited to ULTIPLEAT PROFILE™ filters (Pall Corporation, Port Washington, N.Y.), SUPOR™ membrane filters (Pall Corporation, Port Washington, N.Y.)

In some cases, flow filtration can be carried out to increase filtration speed and/or effectiveness. In some cases, flow filtration can include vacuum filtration. According to such methods, a vacuum is created on the side of the filter opposite that of cell lysate to be filtered. In some cases, cell lysates can be passed through filters by centrifugal forces. In some cases, a pump is used to force cell lysate through clarification filters. Flow rate of cell lysate through one or more filters can be modulated by adjusting one of channel size and/or fluid pressure.

According to some embodiments, cell lysates can be clarified by centrifugation. Centrifugation can be used to pellet insoluble particles in the lysate. During clarification, centrifugation strength (expressed in terms of gravitational units (g), which represents multiples of standard gravitational force) can be lower than in subsequent purification steps. In some cases, centrifugation can be carried out on cell lysates at from about 200 g to about 800 g, from about 500 g to about 1500 g, from about 1000 g to about 5000 g, from about 1200 g to about 10000 g or from about 8000 g to about 15000 g. In some embodiments, cell lysate centrifugation is carried out at 8000 g for 15 minutes. In some cases, density gradient centrifugation can be carried out in order to partition particulates in the cell lysate by sedimentation rate. Gradients used according to methods of the present disclosure can include, but are not limited to cesium chloride gradients and iodixanol step gradients.

In some cases, parvovirus (e.g., AAV) particles comprising one or more capsid protein serotypes and/or sequences of Table 1 can be purified from clarified cell lysates by one or more methods of chromatography. Chromatography refers to any number of methods known in the art for separating out one or more elements from a mixture. Such methods can include, but are not limited to ion exchange chromatography (e.g. cation exchange chromatography and anion exchange chromatography.) immunoaffinity chromatography and size-exclusion chromatography. In some embodiments, methods of viral chromatography can include any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691. WO2000055342, WO2000075353 and WO2001023597, the contents of each of which are herein incorporated by reference by reference in their entirety.

In some embodiments, ion exchange chromatography can be used to isolate parvovirus (e.g., AAV) particles comprising one or more capsid proteins described herein. Ion exchange chromatography is used to bind parvovirus particles based on charge-charge interactions between capsid proteins and charged sites present on a stationary phase, typically a column through which viral preparations (e.g. clarified lysates) are passed. After application of viral preparations, bound parvovirus particles can then be eluted by applying an elution solution to disrupt the charge-charge interactions. Elution solutions can be optimized by adjusting salt concentration and/or pH to enhance recovery of bound parvovirus particles, and can include cation or anion exchange chromatography methods. Methods of ion exchange chromatography can include, but are not limited to any of those taught in U.S. Pat. Nos. 7,419,817, 6,143,548, 7,094,604, 6,593,123, 7,015,026 and 8,137,948, the contents of each of which are herein incorporated by reference in their entirety.

In some embodiments, size-exclusion chromatography (SEC) can be used. SEC can include the use of a gel to separate particles according to size. In parvovirus particle purification, SEC filtration is sometimes referred to as “polishing.” In some cases, SEC can be carried out to generate a final product that is near-homogenous. Such final products can in some cases be used in pre-clinical studies and/or clinical studies (Kotin, R. M. 2011. Human Molecular Genetics. 20(1):R2-R6, the contents of which are herein incorporated by reference in their entirety). In some cases, SEC can be carried out according to any of the methods taught in U.S. Pat. Nos. 6,143,548, 7,015,026, 8,476,418, 6,410,300, 8,476,418, 7,419,817, 7,094,604, 6,593,123, and 8,137,948, the contents of each of which are herein incorporated by reference in their entirety.

In in certain embodiments, parvovirus (e.g., AAV) particles comprising one or more capsid protein serotypes and/or sequences of Table 1 can be isolated or purified using the methods described in U.S. Pat. No. 6,146,874, 6,660,514, 8,283,151, or 8,524,446, the contents of each of which is herein incorporated by reference in its entirety.

### VII. Formulation

### Pharmaceutical Compositions

In one aspect, AAV particles comprising one or more capsid protein serotypes and/or sequences of Table 1 for use in delivery of payloads to a central nervous system region, for example, a brain region, via administration to the CSF may be prepared as pharmaceutical compositions. It will be understood that such compositions necessarily comprise one or more active ingredients and, most often, a pharmaceutically acceptable excipient.

In some embodiments, AAV particle pharmaceutical compositions described herein may comprise at least one payload. As a non-limiting example, the pharmaceutical compositions may contain an AAV particle with 1, 2, 3, 4 or 5 payloads.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, rats, birds, including commercially relevant birds such as poultry, chickens, ducks, geese, and/or turkeys.

In some embodiments, compositions are administered to humans, human patients, or subjects.

### Formulations

Formulations described herein can include, without limitation, saline, liposomes, lipid nanoparticles, polymers, peptides, proteins, cells transfected with viral vectors (e.g., for transfer or transplantation into a subject) and combinations thereof.

Formulations of the pharmaceutical compositions described herein may be prepared by any method known or hereafter developed in the art of pharmacology. As used herein, the term “pharmaceutical composition” refers to compositions comprising at least one active ingredient and optionally one or more pharmaceutically acceptable excipients.

In general, such preparatory methods include the step of associating the active ingredient with an excipient and/or one or more other accessory ingredients. As used herein, the phrase “active ingredient” generally refers either to an AAV particle carrying a payload region encoding the polypeptides described herein or to the end product encoded by a viral genome of an AAV particle as described herein.

Formulations of the AAV particles and pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

In one embodiment, the AAV particles described herein may be formulated in PBS with 0.001% of pluronic acid (F-68) at a pH of about 7.0.

In some embodiments, the AAV formulations described herein may contain sufficient AAV particles for expression of at least one expressed functional payload. As a non-limiting example, the AAV particles may contain viral genomes encoding 1, 2, 3, 4 or 5 functional payloads.

In one aspect, AAV particles may be formulated for CNS delivery. Agents that cross the brain blood barrier may be used. For example, some cell penetrating peptides that can target molecules to the brain blood barrier endothelium may be used for formulation (e.g., Mathupala, Expert Opin Ther Pat., 2009, 19, 137-140; the content of which is incorporated herein by reference in its entirety).

### Excipients and Diluents

The AAV particles described herein can be formulated using one or more excipients or diluents to (1) increase stability (2) increase cell transfection or transduction; (3) permit the sustained or delayed release of the payload; (4) alter the biodistribution (e.g., target the viral particle to specific tissues or cell types); (5) increase the translation of encoded protein; (6) alter the release profile of encoded protein and/or (7) allow for regulatable expression of the payload described herein.

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

### Inactive Ingredients

In some embodiments, AAV particle formulations may comprise at least one inactive ingredient. As used herein, the term “inactive ingredient” refers to one or more agents that do not contribute to the activity of the active ingredient of the pharmaceutical composition included in formulations. In some embodiments, all, none or some of the inactive ingredients which may be used in the formulations described herein may be approved by the US Food and Drug Administration (FDA).

Pharmaceutical composition formulations of AAV particles disclosed herein may include cations or anions. In one embodiment, the formulations include metal cations such as, but not limited to, Zn2+, Ca2+, Cu2+, Mn2+, Mg+ and combinations thereof. As a non-limiting example, formulations may include polymers and complexes with a metal cation (See e.g., U.S. Pat. Nos. 6,265,389 and 6,555,525, each of which is herein incorporated by reference in its entirety).

Formulations described herein may also include one or more pharmaceutically acceptable salts. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids.

The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977); the content of each of which is incorporated herein by reference in their entirety.

The term “pharmaceutically acceptable solvate,” as used herein, means a compound described herein wherein molecules of a suitable solvent are incorporated in the crystal lattice. A suitable solvent is physiologically tolerable at the dosage administered. Solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

### VIII. Methods and Uses of the Compositions of the Disclosure

### Analysis of AAV Capsid Library

In some embodiments, a barcoded AAV library may be used to identify the distribution and transduction pattems of AAV capsid in a subject. The subject may be a mammal, including but not limited to mice, rats, rabbits, non-human primates, and humans. The subject may be a non-human primate (e.g. cynomolgus monkey). The subjects may be pre-screened for the absence of AAV2 and AAV9 neutralizing antibodies via any method known to one skilled in the art.

The barcoded AAV library may be administered to the CNS of a subject using any method described herein. In some embodiments, the AAV library is administered via injection. The injection may be to the cisterna magna. In some embodiments, the barcoded AAV library may be administered to the cerebrospinal fluid via cisternal (CM) administration. Subjects may be administered with a dose of from about 1.0×10¹⁰ vg/kg to about 5.0×10¹⁰ vg/kg, from about 5.0×10¹⁰ vg/kg to about 1.0×10¹¹ vg/kg, from about 1.0×10¹¹ vg/kg to about 5.0×10¹¹ vg/kg, from about 5.0×10¹¹ vg/kg to about 1.0×10¹² vg/kg, from about 1.0×10¹² vg/kg to about 5.0×10¹² vg/kg, from about 5.0×10¹² vg/kg to about 1.0×10¹³ vg/kg, from about 1.0×10¹³ vg/kg to about 5.0×10¹³ vg/kg, from about 5.0×10¹³ vg/kg to about 1.0×10¹⁴ vg/kg, from about 1.0×10¹⁴ vg/kg to about 5.0×10¹⁴ vg/kg, or from about 5.0×10¹⁴ vg/kg to about 1.0×10¹⁵ vg/kg. In some embodiments, the dose administered may be about 4×10¹² vg/kg.

In some embodiments, the DNA-barcoded AAV vector genome is single stranded. In some embodiments, the DNA-barcoded AAV vector genome is double stranded. The AAV vector genome may comprise one or more DNA virus barcodes. In some embodiments, the AAV vector genome may comprise a pair of DNA virus barcodes, as seen in FIG. 1A. The pair of DNA virus barcodes may include a left virus barcode (t-VBC) and a right virus barcode (rt-VBC). The virus barcodes may be up to 5, 6, 7, 8, 9, 10, 11, 12, 13, 14, 15, 16, 17, 18, 19, or 20 nucleotides in length. The virus barcodes may be located downstream of a promoter. That promoter may be a human U6 promoter. Upon infection of cells with AAV vector, the DNA barcodes may be transcribed into RNA barcodes. The virus barcodes may be PCR-amplified independently as either a DNA barcode or an RNA barcode. The Barcode-Seq protocol, as described in Adachi K et at., Nat Commun 5, 3075 (2014), may be used to identify and/or quantify the barcoded samples.

In some embodiments, the barcoded libraries may comprise 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different AAV capsids. In some embodiments, the barcoded library may comprise 58 different AAV capsids, as seen in FIG. 1B. DNA-barcoded AAV vectors, each packaged with a specific AAV capsid, may be produced separately and pooled into one library. For 57 different AAV capsids, there may be 2 unique barcoded clones per capsid. Reference controls, e.g. AAV9, may have 15 unique barcoded clones. In some embodiments, there are a total of 129 different AAV vectors corresponding to 129 unique barcodes in the library. The 58 AAV capsids in an AAV library are listed in Table 1.

After administration of the AAV barcoded libraries, DNA may be isolated from the brain tissue of the subject. The DNA may be isolated up to 1 week, 2 week, 3 weeks, 4 weeks, 5 weeks, 6 weeks, 7 weeks, 8 weeks, 9 weeks, 6 months, or 1 year after administration of the library. In some embodiments, DNA may be isolated 6 weeks after administration. Examples of regions of brain tissue from which DNA may be isolated include, but are not limited to, the frontal cortex, the occipital cortex, the caudate nucleus, the putamen, the thalamus, the hippocampus, the cingulate gyrus, the hypothalamus, the pons, the medulla, the cerebellar Purkinje layer, and the cerebellar granular layer. DNA and RNA may be isolated from the brain tissue and analyzed via any method known to one skilled in the art.

Vector copies may be quantified by any method known to one skilled in the art, including quantitative PCR. In some embodiments, vector genome copies per diploid cell (VG/DC) may be between about 0.01 and about 8.00. In some embodiments, VG/DC may be from about 0.01 to about 1.00, from about 1.00 to about 2.00, from about 2.00 to about 3.00, from about 3.00 to about 4.00, from about 4.00 to about 5.00, from about 5.00 to about 6.00, from about 6.00 to about 7.00, or from about 7.00 to about 8.00. In some embodiments, the highest levels of VG/DC may be present in the medulla, followed by cingulate gyrus, frontal cortex, and occipital cortex. In some embodiments, slightly lower levels of VG/DC may be present in the hypothalamus, hippocampus, pons, cerebellar Purkinje layer, and cerebellar granular layer. In some embodiments, lower levels of VG/DC may be present in the thalamus and caudate nucleus. In some embodiments, the lowest levels of VG/DC may be present in the putamen. In some embodiments, the VG/DC levels in the putamen may be approximately 300-fold lower than in medulla.

In some embodiments. DNA and RNA samples may be subjected to barcode-seq analysis. Barcode-seq analysis may use a sequencing method, such as the Illumina platform (as described in Adachi K et at., Nat Commun 5, 3075 (2014)) to identify and/or quantify the AAV capsids in the sampled tissues and cells. In some embodiments, the relative values of distribution and/or transduction of each AAV capsid compared with AAV9 may be examined in the sampled tissues and cells. In some embodiments, the fold difference in distribution and/or transduction of the AAV capsids, as compared to AAV9, may be from about 0.0 to about 1000.0. In some embodiments, the fold difference in distribution and/or transduction of the AAV capsids, as compared to AAV9, may be from about 0 to about 1, from about 1 to about 5, from about 5 to about 10, from about 10 to about 20, from about 20 to about 50, from about 50 to about 100, from about 100 to about 150, from about 150 to about 200, from about 200 to about 250, from about 250 to about 300, from about 300 to about 350, from about 350 to about 400, from about 400 to about 450, from about 450 to about 500, from about 500 to about 550, from about 550 to about 600, from about 600 to about 650, from about 650 to about 700, from about 700 to about 750, from about 750 to about 800, from about 800 to about 850, from about 850 to about 900, from about 900 to about 950, or from about 950 to about 1000. In some embodiments, an AAV particle described herein shows at least 10-fold higher distribution in a brain region than AAV9. In some embodiments, an AAV particle described herein shows at least 20-fold higher distribution in a brain region than AAV9. In some embodiments, an AAV particle described herein shows at least 50-fold higher distribution in a brain region than AAV9.

In some embodiments, the fold difference in expression of the AAV capsids, as compared to AAV9, may be from about 0 to about 1, from about 1 to about 5, from about 5 to about 10, from about 10 to about 20, from about 20 to about 50, from about 50 to about 100, from about 100 to about 150, from about 150 to about 200, from about 200 to about 250, from about 250 to about 300, from about 300 to about 350, from about 350 to about 400, from about 400 to about 450, from about 450 to about 500, from about 500 to about 550, from about 550 to about 600, from about 600 to about 650, from about 650 to about 700, from about 700 to about 750, from about 750 to about 800, from about 800 to about 850, from about 850 to about 900, from about 900 to about 950, or from about 950 to about 1000. In some embodiments, an AAV particle described herein shows at least 10-fold higher expression in a brain region than AAV9. In some embodiments, an AAV particle described herein shows at least 20-fold higher expression in a brain region than AAV9. In some embodiments, an AAV particle described herein shows at least 50-fold higher expression in a brain region than AAV9.

The distribution and/or transduction of the AAV vectors may be examined in the frontal gyrus, the occipital cortex, the caudate, the putamen, the hippocampus, the cingulate gyrus, the thalamus, the hypothalamus, the pons, the medulla, the cerebellar Purkinje, and/or the cerebellar granular layer. In some embodiments, AAV1, AAV1mt1, AAV2, AAV2mt2, AAV2mt3, AAV2mt4, AAV2mt5, AAV2mt6, AAV2mt7, AAV2mt8, AAV2mt9, AAV2mt10, AAV6mt2, AAV6mt4, AAV6mt5, AAV8, AAV9mt1, AAV9mt6, AAV11, AAVrh10, AAVrh39, AAVDJ and Pig provide greater biodistribution as compared to the control, AAV9. In some embodiments, AAV1, AAV1mt1, AAV2, AAV2mt2, AAV2mt3, AAV2mt4, AAV2mt5, AAV2mt6, AAV2mt7, AAV2mt8, AAV2mt9, AAV2mt10, AAV6mt2, AAV6mt4, AAV6mt5, AAV8, AAV9mt1, AAV9mt6, AAV1, AAVrh10. AAVrh39, AAVDJ and Pig provide greater than 7.5-fold greater biodistribution to multiple regions of the CNS such as the frontal gyrus, occipital cortex, caudate, hippocampus, cingulate gyrus, thalamus, hypothalamus, pons, medulla, cerebellar Purkinje layer, and the cerebellar granular layer.

In some embodiments, AAV9mt6 may have a higher biodistribution in the putamen relative to AAV9.

In some embodiments, AAV2mt2 and AAV2 may have higher biodistribution in the caudate relative to AAV9.

In some embodiments, AAV1 and AAVDJ may have the highest biodistribution, relative to that of AAV9, in the hippocampus and/or the cingulate gyrus.

In some embodiments, AAV2mt2, AAV2mt5, AAV2mt7, AAV2mt8 and AAAVDJ may provide the highest biodistribution, relative to AAV9, in the pons and medulla.

In some embodiments, AAV1, AAV3B, AAV3mt4, AAV6, AAV6mt1, and AAV6mt3 may provide better RNA expression than AAV9 in particular CNS regions. AAV1 may show higher RNA expression than AAV9 in the caudate, thalamus and hypothalamus. AAV3B and AAV3mt4 may provide higher RNA expression than AAV9 in the pons, medulla and cerebellar cortex. AAV6, AAV6mt1, and AAV6mt3 may provide higher RNA expression than AAV9 in the caudate, hippocampus, thalamus, and hypothalamus.

In some embodiments, the analysis of the AAV capsid libraries may identify capsids for delivery of a payload molecule (e.g., a modulatory polynucleotide or a transgene) to any part of the CNS. Exemplary parts of the CNS include, but are not limited to, the frontal cortex, the occipital cortex, the caudate nucleus, the putamen, the thalamus, the hippocampus, the cingulate gyrus, the hypothalamus, the pons, the medulla, the cerebellar Purkinje layer, and the cerebellar granular layer. In some embodiments, the identified capsids may be administered to treat diseases of the CNS and/or part of the CNS.

In some embodiments, the analysis of the AAV capsid libraries may identify capsids for delivery of a payload molecule (e.g. a modulatory polynucleotide or a transgene) to the caudate via CM administration. The identified capsids may include AAV1, AAV6, AAV6mt1, or AAV6mt3. In some embodiments, the identified capsids may be administered to treat diseases involving the caudate. In some embodiments, the disease is Huntington's Disease.

In some embodiments, the analysis of the AAV capsid libraries may identify capsids for delivery of a payload molecule (e.g. a modulatory polynucleotide or a transgene) to the hippocampus via CM administration. The identified capsids may include AAV6 AAV6mt1, or AAV6mt3. In some embodiments, the identified capsids may be administered to treat diseases involving the hippocampus. In some embodiments, the disease is Alzheimer's Disease.

### Neurological Disease

Various neurological diseases may be treated with pharmaceutical compositions and AAV particles comprising one or more capsid protein serotypes and/or sequences of Table 1 described herein. For example, the present disclosure provides a method for treating neurological disorders in a mammalian subject, including a human subject, comprising administering to the subject any of the AAV particles or pharmaceutical compositions described herein. In one embodiment, the AAV particle is a blood brain barrier crossing particle. As a non-limiting example, the neurological disorder may be Absence of the Septum Pellucidum, Acid Lipase Disease, Acid Maltase Deficiency, Acquired Epileptiform Aphasia, Acute Disseminated Encephalomyclitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS-Neurological Complications, Alexander Disease, Alpers' Disease, Alternating Hemiplegia Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder. Autonomic Dysfunction, Back Pain, Barth Syndrome, Batten Disease, Becker's Myotonia, Bechet's Disease, Bell's Palsy, Benign Essential Blepharospasm, Benign Focal Amyotrophy, Benign Intracranial Hypertension, Bemhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury. Brown-Sequard Syndrome, Bulbospinal Muscular Atrophy, Cerebral Autosomal Dominant Arteriopathy with Subcortical Infarcts and Leukoencephalopathy (CADASIL), Canavan Disease, Carpal Tunnel Syndrome, Causalgia, Cavemomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration. Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia—Multi-Infarct, Dementia—Semantic, Dementia—Subcortical, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy (familial infantile), Encephalotrigeminal Angiomatosis, Epilepsy, Epileptic Hemiplegia, Erb's Palsy, Erb-Duchenne and Dejerine-Klumpke Palsies, Essential Tremor, Extrapontine Myelinolysis, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses, Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barré Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritifornis, Herpes Zoster, Herpes Zoster Oticus, Hirayama Syndrome, Holmes-Adic syndrome, Holoprosencephaly, HTLV-1 Associated Myelopathy, Hughes Syndrome, Huntington's Disease, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Keamrn-Sayre Syndrome, Kennedy's Disease, Kinsboume syndrome, Kleine-Levin Syndrome, Klippel-Feil Syndrome, Klippel-Trenaunay Syndrome (KTS), Kluver-Bucy Syndrome, Korsakoffs Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus—Neurological Sequelae, Lyme Disease—Neurological Complications, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia—Congenital, Myasthenia Gravis, Myclinoclastic Diffuse Sclerosis, Myoclonic Encephalopathy of Infants, Myoclonus, Myopathy, Myopathy—Congenital, Myopathy—Throtoxic, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyrne Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy—Hereditary, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain—Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polvmyositis, Pompe Disease, Porncephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of infancy (SMEI), Shaken Baby Syndrome, Shingles, Shy-Drager Syndrome, Sjögren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgifonn (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Von Economo's Disease, Von Hippel-Lindau Disease (VHL), Von Recklinghausen's Disease, Wallenberg's Syndrome, Werdnig-Hoffman Disease, Wemicke-Korsakoff Syndrome, West Syndrome, Whiplash, Whipple's Disease, Williams Syndrome, Wilson Disease, Wolman's Disease, X-Linked Spinal and Bulbar Muscular Atrophy. In some embodiments, neurological disorders treated according to the methods described herein include tauopathies, Alzheimer's disease (AD), Amyotrophic lateral sclerosis (ALS), Huntington's Disease (HD), Parkinson's Disease (PD), and/or Friedreich's Ataxia (FA).

The present disclosure provides a method for administering to a subject in need thereof, including a human subject, a therapeutically effective amount of the AAV particles comprising one or more capsid protein serotypes and/or sequences of Table 1 described herein to slow, stop or reverse disease progression. As a non-limiting example, disease progression may be measured by tests or diagnostic tool(s) known to those skilled in the art. As another non-limiting example, disease progression may be measured by change in the pathological features of the brain, CSF or other tissues of the subject.

In one embodiment, delivery of AAV particles comprising one or more capsid protein serotypes and/or sequences of Table 1 described herein, comprising ApoE2, ApoE3 or ApoE4 polynucleotides, may be used to treat subjects suffering from tauopathy.

In one embodiment, delivery of AAV particles comprising one or more capsid protein serotypes and/or sequences of Table 1 described herein comprising modulatory polynucleotides for the silencing of ApoE2, ApoE3 or ApoE4 gene and/or protein expression may be used to treat subjects suffering from tauopathy.

In one embodiment, delivery of AAV particles comprising one or more capsid protein serotypes and/or sequences of Table 1 described herein comprising modulatory polynucleotides for the silencing of tau gene and/or protein expression may be used to treat subjects suffering from tauopathy.

In one embodiment, delivery of AAV particles described herein comprising a nucleic acid encoding an anti-tau antibody may be used to treat subjects suffering from tauopathy.

In one embodiment, the compositions described herein are used in combination with one or more known or exploratory treatments for tauopathy. Non-limiting examples of such treatments include inhibitors of tau aggregation, such as Methylene blue, phenothiazines, anthraquinones, n-phenylamines or rhodamines, microtubule stabilizers such as NAP, taxol or paclitaxel, kinase or phosphatase inhibitors such as those targeting GSK3β (lithium) or PP2A, and/or immunization with tan phospho-epitopes or treatment with anti-tau antibodies.

In one embodiment, delivery of AAV particles comprising one or more capsid protein serotypes and/or sequences of Table 1 described herein, comprising ApoE2, ApoE3 or ApoE4 polynucleotides, may be used to treat subjects suffering from AD and other tauopathies. In one embodiment, delivery of AAV particles described herein comprising modulatory polynucleotides for the silencing of the ApoE2, ApoE3 or ApoE4 gene and/or protein may be used to treat subjects suffering from AD and other tauopathies. In one embodiment, delivery of AAV particles described herein comprising modulatory polynucleotides for the silencing of the tau gene and/or protein may be used to treat subjects suffering from AD and other tauopathies. In one embodiment, delivery of AAV particles described herein comprising a nucleic acid encoding an anti-tau antibody may be used to treat subjects suffering from AD and other tauopathies.

AAV particles comprising one or more capsid protein serotypes and/or sequences of Table 1 and methods of using the AAV particles described herein may be used to prevent, manage and/or treat ALS. As non-limiting examples, the AAV particles described herein that may be used for the treatment, prevention or management of ALS may comprise modulatory polynucleotides targeting SODL.

AAV particles and methods of using the AAV particles comprising one or more capsid protein serotypes and/or sequences of Table 1 described herein may be used to prevent, manage and/or treat HD. As a non-limiting example, the AAV particles described herein used to treat, prevent and/or manage HD may comprise modulatory polynucleotides targeting HTT.

In some embodiments, methods described herein may be used to treat subjects suffering from PD and other synucleinopathies. In some cases, methods described herein may be used to treat subjects suspected of developing PD and other synucleinopathies such as Parkinson's Disease Dementia (PDD), multiple system atrophy (MSA), dementia with Lewy bodies, juvenile-onset generalized neuroaxonal dystropht (lallervorden-Spatz disease), pure autonomic failure (PAF), neurodegeneration with brain iron accumulation type-1 (NBIA-1) and combined Alzheimer's and Parkinson's Disease.

In one embodiment, delivery of AAV particles comprising one or more capsid protein serotypes and/or sequences of Table 1 described herein, comprising frataxin polynucleotides, may be used to treat subjects suffering from Friedreich's Ataxia. In one embodiment, the AAV particles described herein, comprising frataxin polynucleotides, may be delivered to the dentate nucleus of the cerebellum, brainstem nuclei and/or Clarke's column of the spinal cord. Delivery to one or more of these regions may treat and/or reduce the effects of Friedreich's Ataxia in a subject.

### Methods of Treatment of Neurological Disease

### AAV Particles Encoding Protein Payloads

In one aspect, disclosed herein are methods for treating neurological disease associated with insufficient function/presence of a target protein (e.g., ApoE, FXN) in a subject in need of treatment. The method optionally comprises administering to the subject a therapeutically effective amount of a composition comprising AAV particles comprising one or more capsid protein serotypes and/or sequences of Table 1 described herein. As a non-limiting example, the AAV particles can increase target gene expression, increase target protein production, and thus reduce one or more symptoms of neurological disease in the subject such that the subject is therapeutically treated.

In one aspect, the composition comprising the AAV particles described herein that comprise a capsid protein serotype and/or sequence of Table 1 is administered to the central nervous system, for example, a brain region, of the subject via administration to the CSF.

In one embodiment, the composition comprising the AAV particles described herein that comprise a capsid protein serotype and/or sequence of Table 1 is administered to the central nervous system, for example a brain region, of the subject via intraparenchymal injection. Non-limiting examples of intraparenchymal injections include intrathalamic, intrastriatal, intrahippocampal or targeting the entorhinal cortex.

In one embodiment, the composition comprising the AAV particles described herein that comprise a capsid protein serotype and/or sequence of Table 1 is administered to the central nervous system of the subject via intraparenchymal injection and intrathecal injection.

In one embodiment, the AAV particles described herein that comprise a capsid protein serotype and/or sequence of Table 1 may be delivered into specific types of targeted cells, including, but not limited to, hippocampal, cortical, motor or entorhinal neurons; glial cells including oligodendrocytes, astrocytes and microglia; and/or other cells surrounding neurons such as T cells. In one embodiment, the AAV particles described herein that comprise a capsid protein serotype and/or sequence of Table 1 may be delivered into a cell of the frontal gyrus, occipital cortex, caudate, putamen, hippocampus, cingulate gyrus, thalamus, hypothalamus, cerebellar Purkinje, or cerebellar granular layer. In one embodiment, the AAV particles described herein that comprise a capsid protein serotype and/or sequence of Table 1 may be delivered to neurons in the striatum and/or cortex.

In some embodiments, the AAV particles described herein that comprise a capsid protein serotype and/or sequence of Table 1 may be used as a therapy for neurological disease.

In some embodiments, the AAV particles described herein that comprise a capsid protein serotype and/or sequence of Table 1 may be used as a therapy for tauopathies.

In some embodiments, the AAV particles described herein that comprise a capsid protein serotype and/or sequence of Table 1 may be used as a therapy for Alzheimer's Disease.

In some embodiments, the AAV particles described herein that comprise a capsid protein serotype and/or sequence of Table 1 may be used as a therapy for Amyotrophic Lateral Sclerosis.

In some embodiments, the AAV particles described herein that comprise a capsid protein serotype and/or sequence of Table 1 may be used as a therapy for Huntington's Disease.

In some embodiments, the AAV particles described herein that comprise a capsid protein serotype and/or sequence of Table 1 may be used as a therapy for Parkinson's Disease.

In some embodiments, the AAV particles described herein that comprise a capsid protein serotype and/or sequence of Table 1 may be used as a therapy for Friedreich's Ataxia.

In some embodiments, the AAV particles described herein that comprise a capsid protein serotype and/or sequence of Table 1 may be used to increase target protein expression in astrocytes in order to treat a neurological disease. Target protein in astrocytes may be increased by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the AAV particles may be used to increase target protein in microglia. The increase of target protein in microglia may be, independently, increased by 5%, 10%, 15%, 20%, 25% 30%, 35%, 40%, 45%, 50%, 55%, 60%, 65%, 70%, 75%, 80%, 85%, 90%, 95%, or more than 95%.

In some embodiments, the AAV particles may be used to increase target protein in cortical neurons. The increase of target protein in the cortical neurons may be, independently, increased by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the AAV particles may be used to increase target protein in hippocampal neurons. The increase of target protein in the hippocampal neurons may be, independently, increased by 5%, 10%, 15%, 20%, 25%, 30%, 35%, 40%, 45%, 50%, 55%, 60%, 65%, 70%, 75%, 80%, 85%, 90%, 95%, or more than 95

In some embodiments, the AAV particles may be used to increase target protein in DRG and/or sympathetic neurons. The increase of target protein in the DRG and/or sympathetic neurons may be, independently, increased by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the AAV particles described herein may be used to increase target protein in sensory neurons in order to treat neurological disease. Target protein in sensory neurons may be increased by 5%, 10%, 15%, 20%, 25%, 30%, 35%, 40%, 45%, 50%, 55%, 60%, 65%, 70%, 75%, 80%, 85%, 90%, 95%, or more than 95%.

In one embodiment, administration of the AAV particles described herein that comprise a capsid protein serotype and/or sequence of Table 1 to a subject may increase target protein levels in a subject. The target protein levels may be increased by about 10%, 20%, 30%, 40%, 50%, 60%, 70%, 80%, 85%, 90%, 95% and 100% in a subject such as, but not limited to, the CNS, a region of the CNS, or a specific cell of the CNS of a subject. As a non-limiting example, a subject may have an increase of 10% of target protein. As a non-limiting example, the AAV particles may increase the protein levels of a target protein by fold increases over baseline. In one embodiment. AAV particles lead to 5-6 times higher levels of a target protein.

In one embodiment, administration of the AAV particles described herein that comprise a capsid protein serotype and/or sequence of Table 1 to a subject may increase the expression of a target protein in a subject. The expression of the target protein may be increased by about 10%, 20%, 30%, 40%, 50%, 60%, 70%, 80%, 85%, 90%, 95% and 100% in a subject such as, but not limited to, the CNS, a region of the CNS, or a specific cell of the CNS of a subject.

In one embodiment, administration of the AAV particles that comprise a capsid protein serotype and/or sequence of Table 1 to a subject will increase the expression of a target protein in a subject and the increase of the expression of the target protein will reduce the effects and/or symptoms of neurological disease in a subject.

### AAV Particles Comprising Modulatory Polynucleotides

In one aspect, provided herein are methods for introducing the AAV particles described herein that comprise a capsid protein serotype and/or sequence of Table 1, comprising a nucleic acid sequence encoding the siRNA molecules described herein into cells, the method comprising introducing into said cells any of the vectors in an amount sufficient for degradation of a target mRNA to occur, thereby activating target-specific RNAi in the cells. In some aspects, the cells may be neurons such as but not limited to, motor, hippocampal, entorhinal, thalamic or cortical neurons, and glial cells such as astrocytes or microglia. In certain embodiments, the cells may be cells of the frontal gyrus, occipital cortex, caudate, putamen, hippocampus, cingulate gyrus, thalamus, hypothalamus, cerebellar Purkinje, or cerebellar granular layer.

In one aspect, provided herein are methods for treating neurological diseases associated with dysfunction of a target protein in a subject in need of treatment. The method optionally comprises administering to the subject a therapeutically effective amount of a composition comprising AAV particles described herein that comprise a capsid protein serotype and/or sequence of Table 1 comprising a nucleic acid sequence encoding the siRNA molecules described herein. As a non-limiting example, the siRNA molecules can silence target gene expression, inhibit target protein production, and reduce one or more symptoms of neurological disease in the subject such that the subject is therapeutically treated.

In some embodiments, the composition comprising the AAV particles described herein that comprise a capsid protein serotype and/or sequence of Table 1 comprising a nucleic acid sequence encoding the siRNA molecules described herein is administered to the central nervous system of the subject, for example, a brain region of the subject.

In one embodiment, the composition comprising the AAV particles comprising a capsid protein serotype and/or sequence of Table 1 and a nucleic acid sequence encoding the siRNA molecules described herein is administered to the central nervous system of the subject via intrathecal injection.

In one embodiment, the AAV particles comprising a capsid protein of Table 1 and a nucleic acid sequence encoding the siRNA molecules described herein may be delivered into specific types of targeted cells, including, but not limited to, hippocampal, cortical, motor or entorhinal neurons; glial cells including oligodendrocytes, astrocytes and microglia; and/or other cells surrounding neurons such as T cells. In some embodiments, the cells are cells of the frontal cortex, occipital cortex, caudate nucleus, putamen, thalamus, hippocampus, cingulate gyrus, hypothalamus, pons, medulla, cerebellar Purkinje layer, and cerebellar granular layer.

In one embodiment, the AAV particles comprising a capsid protein serotype and/or sequence of Table 1 and a nucleic acid sequence encoding the siRNA molecules described herein may be delivered to neurons in the striatum and/or cortex.

In some embodiments, the AAV particles comprising a capsid protein serotype and/or sequence of Table 1 and a nucleic acid sequence encoding the siRNA molecules described herein may be used as a therapy for neurological disease.

In some embodiments, the AAV particles comprising a capsid protein serotype and/or sequence of Table 1 and a nucleic acid sequence encoding the siRNA molecules described herein may be used as a therapy for tauopathies.

In some embodiments, the AAV particles comprising a capsid protein serotype and/or sequence of Table 1 and a nucleic acid sequence encoding the siRNA molecules described herein may be used as a therapy for Alzheimer's Disease.

In some embodiments, the AAV particles comprising a capsid protein serotype and/or sequence of Table 1 and a nucleic acid sequence encoding the siRNA molecules described herein may be used as a therapy for Amyotrophic Lateral Sclerosis.

In some embodiments, the AAV particles comprising a capsid protein serotype and/or sequence of Table 1 and a nucleic acid sequence encoding the siRNA molecules described herein may be used as a therapy for Huntington's Disease.

In some embodiments, the AAV particles comprising a capsid protein serotype and/or sequence of Table 1 and a nucleic acid sequence encoding the siRNA molecules described herein may be used as a therapy for Parkinson's Disease.

In some embodiments, the AAV particles comprising a n capsid protein serotype and/or sequence of Table 1 and a nucleic acid sequence encoding the siRNA molecules described herein may be used as a therapy for Friedreich's Ataxia.

In some embodiments, the AAV particles comprising a capsid protein serotype and/or sequence of Table 1 and a nucleic acid sequence encoding the siRNA molecules described herein may be used to suppress a target protein in astrocytes in order to treat neurological disease. Target protein in astrocytes may be suppressed by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the AAV particles comprising a capsid protein serotype and/or sequence of Table 1 and a nucleic acid sequence encoding the siRNA molecules described herein may be used to suppress a target protein in microglia. The suppression of the target protein in microglia may be, independently, suppressed by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the AAV particles comprising a capsid protein serotype and/or sequence of Table 1 and a nucleic acid sequence encoding the siRNA molecules described herein may be used to suppress target protein in cortical neurons. The suppression of a target protein in cortical neurons may be, independently, suppressed by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the AAV particles comprising a capsid protein serotype and/or sequence of Table 1 and a nucleic acid sequence encoding the siRNA molecules described herein may be used to suppress a target protein in hippocampal neurons. The suppression of a target protein in the hippocampal neurons may be, independently, suppressed by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the AAV particles comprising a capsid protein serotype and/or sequence of Table 1 and a nucleic acid sequence encoding the siRNA molecules described herein may be used to suppress a target protein in DRG and/or sympathetic neurons. The suppression of a target protein in the DRG and/or sympathetic neurons may be, independently, suppressed by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the AAV particles comprising a capsid protein serotype and/or sequence of Table 1 and a nucleic acid sequence encoding the siRNA molecules described herein may be used to suppress a target protein in sensory neurons in order to treat neurological disease. Target protein in sensory neurons may be suppressed by 5%, 10%, 15%, 20%, 25%, 30%, 35%, 40%, 45%, 50%, 55%, 60%, 65%, 70%, 75%, 80%, 85%, 90%, 95%, or more than 95%.

In some embodiments, the AAV particles comprising a capsid protein serotype and/or sequence of Table 1 and a nucleic acid sequence encoding the siRNA molecules described herein may be used to suppress a target protein and reduce symptoms of neurological disease in a subject. The suppression of target protein and/or the reduction of symptoms of neurological disease may be, independently, reduced or suppressed by 5^{%}, 10%, 15%, 20%, 25%, 30%, 35%, 40%, 45%, 50%, 55%, 60%, 65%, 70%, 75%, 80%, 85%, 90%, 95%, or more than 95%.

In some embodiments, the present composition comprising an AAV particle that comprises a capsid protein serotype and/or sequence of Table 1, is administered as a solo therapeutic or as combination therapeutic for the treatment of neurological disease.

The AAV particles described herein comprising a capsid protein serotype and/or sequence of Table 1 and encoding siRNA duplexes targeting the gene of interest may be used in combination with one or more other therapeutic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent.

Therapeutic agents that may be used in combination with the AAV particles comprising a capsid protein serotype and/or sequence of Table 1 encoding the nucleic acid sequence for the siRNA molecules described herein can be small molecule compounds which are antioxidants, anti-inflammatory agents, anti-apoptosis agents, calcium regulators, antiglutamatergic agents, structural protein inhibitors, compounds involved in muscle function, and compounds involved in metal ion regulation.

In some embodiments, the composition described herein for treating neurological disease is administered to the subject in need intrathecally and/or intraventricularly, allowing the siRNA molecules or vectors comprising the siRNA molecules to be distributed via CSF. The vectors may be used to silence or suppress target gene expression, and/or reducing one or more symptoms of neurological disease in the subject such that the subject is therapeutically treated.

In one embodiment, administration of the AAV particles comprising a capsid protein serotype and/or sequence of Table 1 encoding a siRNA described herein, to a subject may lower target protein levels in a subject. The target protein levels may be lowered by about 10%, 20%, 50%, 60%, 70%, 80%, 85%, 90%, 95% and 100%.

In one embodiment, administration of the AAV particles comprising a capsid protein serotype and/or sequence of Table 1 encoding a siRNA described herein, to a subject may lower the expression of a target protein in a subject. The expression of a target protein may be lowered by about 10%, 20%, 30%, 40%, 50%, 60%, 70%, 80%, 85%, 90%, 95% and 100% in a subject such as, but not limited to, the CNS, a region of the CNS, or a specific cell of the CNS of a subject.

In one embodiment, administration of the AAV particles comprising a capsid protein serotype and/or sequence of Table 1 encoding a siRNA described herein, to a subject may lower the expression of a target protein in the CNS of a subject. The expression of a target protein may be lowered by about 10%, 20%, 30%, 40%, 50%, 60%, 70%, 80%, 85%, 90%, 95% and 100% in a subject.

In one embodiment, administration of the AAV particles described comprising a capsid protein serotype and/or sequence of Table 1 to a subject will reduce the expression of a target protein in a subject and the reduction of expression of the target protein will reduce the effects and/or symptoms of neurological disease in a subject.

In one embodiment, the AAV particles described comprising a capsid protein serotype and/or sequence of Table 1 may be used to decrease target protein in a subject. The decrease may independently be 5%, 10%, 15%, 20%, 25%, 30%, 35%, 40%, 45%, 50%, 55%, 60%, 65%, 70%, 75%, 80%, 85%, 90%, 95%, or more than 95%.

### IX. Definitions

At various places in the present specification, substituents of compounds of the present disclosure are disclosed in groups or in ranges. It is specifically intended that the present disclosure include each and every individual subcombination of the members of such groups and ranges.

Unless stated otherwise, the following terms and phrases have the meanings described below. The definitions are not meant to be limiting in nature and serve to provide a clearer understanding of certain aspects described herein.

About: As used herein, the term “about” means +/−10% of the recited value.

Adeno-associated virus: The term “adeno-associated virus” or “AAV” as used herein refers to members of the dependovius genus comprising any particle, sequence, gene, protein, or component derived therefrom.

AAVP article: As used herein, an “AAV particle” is a virus which comprises a capsid and a viral genome with at least one payload region and at least one ITR region. AAV particles of the present disclosure may be produced recombinantly and may be based on adeno-associated virus (AAV) parent or reference sequences. Generally, the AAV particles described herein comprise one or more capsid protein serotypes and/or sequences of Table 1. The AAV particle may be replication defective and/or targeted.

Activity: As used herein, the term “activity” refers to the condition in which things are happening or being done. Compositions described herein may have activity and this activity may involve one or more biological events.

Administering: As used herein, the term “administering” refers to providing a pharmaceutical agent or composition to a subject.

Administered in combination: As used herein, the term “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of neurodegeneration disorder, amelioration includes the reduction of neuron loss.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Antisense strand: As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Barcode: As used herein, the term “barcode” refers to a representational pattern or marker that is unique to the subject which it encodes. The subject which it encodes may be the identity of a vector genome and/or AAV particle. The representational pattern may comprise units of a polymer. The units may include, but are not limited to, amino acids and nucleic acids. Nucleic acids may include deoxyribonucleic acid (DNA) or ribonucleic acid (RNA). Barcodes may be up to 5, 6, 7, 8, 9, 10, 11, 12, 13, 14, 15, 16, 17, 18, 19, or 20 units in length.

Barcode-seq: As used herein, the term “barcode-seq” refers to the technique of sequencing with uniquely tagged AAV viral genomes for the identification of AAV with the desired properties. The sequencing methods may include next generation sequencing.

Bifunctional: As used herein, the term “bifunctional” refers to any substance, molecule or moiety which is capable of or maintains at least two functions. The functions may affect the same outcome or a different outcome. The structure that produces the function may be the same or different.

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, an AAV particle described herein may be considered biologically active if even a portion of the encoded payload is biologically active or mimics an activity considered biologically relevant.

Central nervous system: As used herein, the term “central nervous system” or “CNS” refers to the tissues that control and coordinate the flow of information throughout the body of an organism. The central nervous system comprises nerve tissues, and it includes the brain and the spinal cord.

Cisterna magna: As used herein, the term “cisterna magna” refers to an area of the brain. This area comprises an opening in the subarachnoid space found between the cerebellum and the dorsal surface of the medulla oblongata.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U. C to G), or in any other manner that allows for the formation of duplexes. As persons skilled in the art are aware, when using RNA as opposed to DNA, uracil rather than thymine is the base that is considered to be complementary to adenosine. However, when a U is denoted in the context described herein, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity. As used herein, the term “substantially complementary” means that the siRNA has a sequence (e.g., in the antisense strand) which is sufficient to bind the desired target mRNA, and to trigger the RNA silencing of the target mRNA.

Compound: Compounds of the present disclosure include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

Conditionally active: As used herein, the term “conditionally active” refers to a mutant or variant of a wild-type polypeptide, wherein the mutant or variant is more or less active at physiological conditions than the parent polypeptide. Further, the conditionally active polypeptide may have increased or decreased activity at aberrant conditions as compared to the parent polypeptide. A conditionally active polypeptide may be reversibly or irreversibly inactivated at normal physiological conditions or aberrant conditions.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an polynucleotide or polypeptide or may apply to a portion, region or feature thereof.

Control Elements: As used herein, “control elements”, “regulatory control elements” or “regulatory sequences” refers to promoter regions, polyadenylation signals, transcription termination sequences, upstream regulatory domains, origins of replication, internal ribosome entry sites (“IRES”), enhancers, and the like, which provide for the replication, transcription and translation of a coding sequence in a recipient cell. Not all of these control elements need always be present as long as the selected coding sequence is capable of being replicated, transcribed and/or translated in an appropriate host cell.

Controlled Release: As used herein, the term “controlled release” refers to a pharmaceutical composition or compound release profile that conforms to a particular pattern of release to effect a therapeutic outcome.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivery: As used herein, “delivery” refers to the act or manner of delivering an AAV particle, a compound, substance, entity, moiety, cargo or payload.

Delivery Agent: As used herein. “delivery agent” refers to any substance which facilitates, at least in part, the in vivo delivery of an AAV particle to targeted cells.

Destabilized: As used herein, the term “destable,” “destabilize,” or “destabilizing region” means a region or molecule that is less stable than a starting, wild-type or native form of the same region or molecule.

Detectable label: As used herein “detectable label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the peptides or proteins disclosed herein. They may be within the amino acids, the peptides, or proteins, or located at the N- or C-termini.

Digest: As used herein, the term “digest” means to break apart into smaller pieces or components. When referring to polypeptides or proteins, digestion results in the production of peptides.

Distal: As used herein, the term “distal” means situated away from the center or away from a point or region of interest.

Dosing regimen: As used herein, a “dosing regimen” is a schedule of administration or physician determined regimen of treatment, prophylaxis, or palliative care.

Encapsulate: As used herein, the term “encapsulate” means to enclose, surround or encase.

Engineered: As used herein, embodiments described herein are “engineered” when they are designed to have a feature or property, whether structural or chemical, that varies from a starting point, wild type or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” includes at least one AAV particle and a delivery agent.

Fragment: A “fragment,” as used herein, refers to a portion. For example, fragments of proteins may comprise polypeptides obtained by digesting full-length protein isolated from cultured cells.

Functional: As used herein, a “functional” biological molecule is a biological molecule in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Homology: As used herein, the term “homology” refers to the overall relatedness between polymeric molecules, e.g. between polynucleotide molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In one aspect, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In one aspect, two protein sequences are considered to be homologous if the proteins are at least about 50%, 60%, 70%, 80%, or 90% identical for at least one stretch of at least about 20 amino acids.

Heterologous Region: As used herein the term “heterologous region” refers to a region which would not be considered a homologous region.

Homologous Region: As used herein the term “homologous region” refers to a region which is similar in position, structure, evolution origin, character, form or function.

Identity: As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology. Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer. Gribskov, M, and Devereux, J., eds., M Stockton Press, New York, 1991: each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Substantially isolated: By “substantially isolated” is meant that a substance is substantially separated from the environment in which it was formed or detected. Partial separation can include, for example, a composition enriched in the substance or AAV particles of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Library: As used herein, the term “library” refers to a collection of viral genomes and/or AAV particles with varying properties. This collection may comprise 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 different AAV capsids. In some embodiments, libraries may comprise hundreds, thousands, or millions of different AAV capsids.

Linker: As used herein “linker” refers to a molecule or group of molecules which connects two molecules. A linker may be a nucleic acid sequence connecting two nucleic acid sequences encoding two different polypeptides. The linker may or may not be translated. The linker may be a cleavable linker.

MicroRNA (miRNA) binding site: As used herein, a microRNA (miRNA) binding site represents a nucleotide location or region of a nucleic acid transcript to which at least the “seed” region of a miRNA binds.

Modified: As used herein “modified” refers to a changed state or structure of a molecule described herein. Molecules may be modified in many ways including chemically, structurally, and functionally.

Mutation: As used herein, the term “mutation” refers to any changing of the structure of a gene, resulting in a variant (also called “mutant”) form that may be transmitted to subsequent generations. Mutations in a gene may be caused by the alternation of single base in DNA, or the deletion, insertion, or rearrangement of larger sections of genes or chromosomes.

Naturally Occurring: As used herein, “naturally occurring” or “wild-type” means existing in nature without artificial aid, or involvement of the hand of man.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Of-target: As used herein, “off target” refers to any unintended effect on any one or more target, gene, or cellular transcript.

Open reading frame: As used herein, “open reading frame” or “ORF” refers to a sequence which does not contain a stop codon in a given reading frame.

Operably linked: As used herein, the phrase “operably linked” refers to a functional connection between two or more molecules, constructs, transcripts, entities, moieties or the like.

Particle: As used herein, a “particle” is a virus comprised of at least two components, a protein capsid and a polynucleotide sequence enclosed within the capsid.

Patient: As used herein, “patient” refers to a subject who may seek or be in need of treatment, requires treatment, is receiving treatment, will receive treatment, or a subject who is under care by a trained professional for a particular disease or condition.

Payload: As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide or a modulatory nucleic acid or regulatory nucleic acid.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound described herein wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacokinetic: As used herein “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Preventing: As used herein, the term “preventing” or “prevention” refers to partially or completely delaying onset of an infection, disease, disorder and/or condition: partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease.

Protein of interest: As used herein, the terms “proteins of interest” or “desired proteins” include those provided herein and fragments, mutants, variants, and alterations thereof.

Proximal: As used herein, the term “proximal” means situated nearer to the center or to a point or region of interest.

Purified: As used herein, “purify,” “purified,” “purification” means to make substantially pure or clear from unwanted components, material defilement, admixture or imperfection. “Purified” refers to the state of being pure. “Purification” refers to the process of making pure.

Region: As used herein, the term “region” refers to a zone or general area. In some embodiments, when referring to a protein or protein module, a region may comprise a linear sequence of amino acids along the protein or protein module or may comprise a three-dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini. N-termini refer to the end of a protein comprising an amino acid with a free amino group. C-termini refer to the end of a protein comprising an amino acid with a free carboxyl group. N- and/or C-terminal regions may there for comprise the N- and/or C-termini as well as surrounding amino acids. In some embodiments, N- and/or C-terminal regions comprise from about 3 amino acid to about 30 amino acids, from about 5 amino acids to about 40 amino acids, from about 10 amino acids to about 50 amino acids, from about 20 amino acids to about 100 amino acids and/or at least 100 amino acids. In some embodiments, N-terminal regions may comprise any length of amino acids that includes the N-terminus, but does not include the C-terminus. In some embodiments, C-terminal regions may comprise any length of amino acids, which include the C-terminus, but do not comprise the N-terminus.

In some embodiments, when referring to a polynucleotide, a region may comprise a linear sequence of nucleic acids along the polynucleotide or may comprise a three-dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and 3′ termini. 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for comprise the 5′ and 3′ termini as well as surrounding nucleic acids. In some embodiments, 5′ and 3′ terminal regions comprise from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In some embodiments, 5′ regions may comprise any length of nucleic acids that includes the 5′ terminus, but does not include the 3′ terminus. In some embodiments, 3′ regions may comprise any length of nucleic acids, which include the 3′ terminus, but does not comprise the 5′ terminus.

RNA or RNA molecule: As used herein, the term “RNA” or “RNA molecule” or “ribonucleic acid molecule” refers to a polymer of ribonucleotides: the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

RNA interfering or RNAi: As used herein, the term “RNA interfering” or “RNA” refers to a sequence specific regulatory mechanism mediated by RNA molecules which results in the inhibition or interfering or “silencing” of the expression of a corresponding protein-coding gene. RNAi has been observed in many types of organisms, including plants, animals and fungi. RNAi occurs in cells naturally to remove foreign RNAs (e.g., viral RNAs). Natural RNAi proceeds via fragments cleaved from free dsRNA which direct the degradative mechanism to other similar RNA sequences. RNAi is controlled by the RNA-induced silencing complex (RISC) and is initiated by short/small dsRNA molecules in cell cytoplasm, where they interact with the catalytic RISC component argonaute. The dsRNA molecules can be introduced into cells exogenously. Exogenous dsRNA initiates RNAi by activating the ribonuclease protein Dicer, which binds and cleaves dsRNAs to produce double-stranded fragments of 21-25 base pairs with a few unpaired overhang bases on each end. These short double stranded fragments are called small interfering RNAs (siRNAs).

Sample: As used herein, the term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Self-complementary viral particle: As used herein, a “self-complementary viral particle” is a particle comprised of at least two components, a protein capsid and a polynucleotide sequence encoding a self-complementary genome enclosed within the capsid.

Sense Strand: As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” includes a siRNA strand having sufficient complementarity to a section of about 10-50 nucleotides of the mRNA of the gene targeted for silencing and a siRNA strand having sufficient complementarity to form a duplex with the other siRNA strand.

Short interfering RNA or siRNA: As used herein, the terms “short interfering RNA,” “small interfering RNA” or “siRNA” refer to an RNA molecule (or RNA analog) comprising between about 5-60 nucleotides (or nucleotide analogs) which is capable of directing or mediating RNAi. Preferably, a siRNA molecule comprises between about 15-30 nucleotides or nucleotide analogs, such as between about 16-25 nucleotides (or nucleotide analogs), between about 18-23 nucleotides (or nucleotide analogs), between about 19-22 nucleotides (or nucleotide analogs) (e.g., 19, 20, 21 or 22 nucleotides or nucleotide analogs), between about 19-25 nucleotides (or nucleotide analogs), and between about 19-24 nucleotides (or nucleotide analogs). The term “short” siRNA refers to a siRNA comprising 5-23 nucleotides, preferably 21 nucleotides (or nucleotide analogs), for example, 19, 20, 21 or 22 nucleotides. The term “long” siRNA refers to a siRNA comprising 24-60 nucleotides, preferably about 24-25 nucleotides, for example, 23, 24, 25 or 26 nucleotides. Short siRNAs may, in some instances, include fewer than 19 nucleotides, e.g., 16, 17 or 18 nucleotides, or as few as 5 nucleotides, provided that the shorter siRNA retains the ability to mediate RNAi. Likewise, long siRNAs may, in some instances, include more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA, siRNAs can be single stranded RNA molecules (ss-siRNAs) or double stranded RNA molecules (ds-siRNAs) comprising a sense strand and an antisense strand which hybridized to form a duplex structure called siRNA duplex.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact, i.e., single administration event. In some embodiments, a single unit dose is provided as a discrete dosage form (e.g., a tablet, capsule, patch, loaded syringe, vial, etc.).

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Spit dose: As used herein, a “split dose” is the division of single unit dose or total daily dose into two or more doses.

Stable: As used herein “stable” refers to a compound that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition: (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synthetic: The term “synthetic” means produced, prepared, and/or manufactured by the hand of man. Synthesis of polynucleotides or polypeptides or other molecules described herein may be chemical or enzymatic.

Targeting: As used herein, “targeting” means the process of design and selection of nucleic acid sequence that will hybridize to a target nucleic acid and induce a desired effect.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24 hour period. It may be administered as a single unit dose.

Transfection: As used herein, the term “transfection” refers to methods to introduce exogenous nucleic acids into a cell. Methods of transfection include, but are not limited to, chemical methods, physical treatments and cationic lipids or mixtures.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Vector: As used herein, a “vector” is any molecule or moiety which transports, transduces or otherwise acts as a carrier of a heterologous molecule. Vectors described herein may be produced recombinantly and may be based on and/or may comprise adeno-associated virus (AAV) parent or reference sequence. Such parent or reference AAV sequences may serve as an original, second, third or subsequent sequence for engineering vectors. In non-limiting examples, such parent or reference AAV sequences may comprise any one or more of the following sequences: a polynucleotide sequence encoding a polypeptide or multi-polypeptide, which sequence may be wild-type or modified from wild-type and which sequence may encode full-length or partial sequence of a protein, protein domain, or one or more subunits of a protein; a polynucleotide comprising a modulatory or regulatory nucleic acid which sequence may be wild-type or modified from wild-type; and a transgene that may or may not be modified from wild-type sequence. These AAV sequences may serve as either the “donor” sequence of one or more codons (at the nucleic acid level) or amino acids (at the polypeptide level) or “acceptor” sequences of one or more codons (at the nucleic acid level) or amino acids (at the polypeptide level).

Viral genome: As used herein, a “viral genome” or “vector genome” is a polynucleotide comprising at least one inverted terminal repeat (ITR) and at least one encoded payload. A viral genome encodes at least one copy of the payload.

### Examples

### Example 1. Non-Human Primate Study

To investigate the distribution and transduction patterns of various AAV capsids in nonhuman primate (NHP) central nervous system (CNS) after cisterna magna (CM) injection, a barcoded AAV library was generated with 58 different AAV capsids, including AAV9 as a reference control (FIG. 1B). FIG. 1A shows a map of a DNA-barcoded AAV genome. The AAV vector genome contains a pair of DNA virus barcodes (VBC) (left virus barcode (lt-VBC) and right virus barcode (rt-VBC)), each 12 nucleotides in length, that were placed downstream of the human U6 promoter. After cells were infected with AAV vector, the DNA virus barcodes were transcribed into corresponding RNA VBC driven by the U6 promoter. Each VBC can be PCR-amplified independently, whether as a DNA VBC or RNA VBC. The Barcode-Seq procedure, similar to that described previously (Adachi K et at., Nat Commun 5, 3075 (2014); incorporated by reference in its entirety), was applied for quantifying each VBC.

DNA-barcoded AAV vectors and AAV capsids were produced separately and pooled into one library (barcoded AAV library). There was a total of 129 different AAV vectors tested in 58 different capsids. For the control (AAV9), there were 15 unique barcoded clones tested in separate AAV vectors. The 58 AAV capsids in the library are listed in Table 1. For the rest of the capsids, there were 2 unique barcoded clones tested for each capsid (a total of 114 AAV vectors).

Two male cynomolgus monkeys approximately 3 kg each, were pre-screened for the absence of AAV2 and AAV9 neutralizing antibodies using a cell-based in vitro assay, and then were administered the barcoded AAV library via cisternal (CM) administration into the cerebrospinal fluid at a dose of 4×10¹² vg/kg. Six weeks post-dosing, animals were perfused with cold saline, and the brains were removed and sectioned coronally into slabs of 6 mm thickness using a brain matrix. Brain slabs from the left hemisphere were cut into 6×7×7 mm³ cubes, resulting in a total 111 cubes from 8 slices and 115 cubes from 9 slices from animals #1 and #2, respectively. Twelve specific brain regions (frontal cortex, occipital cortex, caudate nucleus, putamen, thalamus, hippocampus, cingulate gyrus, hypothalamus, pons, medulla, cerebellar Purkinje layer, and cerebellar granular layer) were dissected from the brain slabs from the right hemisphere. Each tissue sample was minced, then lysed. Total DNA was isolated from tissue lysates using KingFisher™ Cell and Tissue DNA Kit (Thermo Fisher Scientific; 97030196) with the automated magnetic processor KingFisher Flex. For RNA isolation, minced tissue was lysed with 2 ml Hard Tissue Homogenizing Mix Tube (Fisher Scientific; 15-340-154) in Trizol reagent using the Bead Mill 24 Homogenizer (Fisher Scientific). Total RNA was isolated from the upper aqueous phase after chloroform extraction using Mag-Bind Total RNA 96 Kit (OMEGA Bio-teck; M6731-01) with the KingFisher Flex instrument.

To quantify vector genome copy number, quantitative PCR was performed using 100 ng total DNA extracted from each brain region sample, and a barcoded AAV vector genome-specific primer set with a 2× Power SYBR Green master mix in a 25 μl reaction. The beta actin gene was used as an internal control for normalization. Vector genome copies per diploid cell (VG/DC) are shown in Table 4. Highest levels of VG/DC were present in the medulla, followed by cingulate gyrus, frontal cortex, and occipital cortex. Slightly lower levels of VG/DC were present in the hypothalamus, hippocampus, pons, cerebellar Purkinje layer, and cerebellar granular layer. Lower levels were present in the thalamus and caudate nucleus, and lowest levels (approximately 300-fold lower than in medulla) were present in the putamen.

TABLE 4
Vector Genome (VG/DC) Distribution in Brain After CM Injection

| Brain Region | Animal #1 | Animal #2 |
|---|---|---|
| Frontal Cortex | 0.90 | 0.91 |
| Occipital Cortex | 0.56 | 1.08 |
| Caudate Nucleus | 0.04 | 0.08 |
| Putamen | 0.01 | 0.01 |
| Thalamus | 0.16 | 0.02 |
| Hippocampus | 0.60 | 0.18 |
| Cingulate Gyrus | 1.88 | 1.00 |
| Hypothalamus | 1.28 | 0.28 |
| Pons | 0.68 | 0.24 |
| Medulla | 7.95 | 3.24 |
| Cerebellar Purkinje | 0.25 | 0.32 |
| layer | | |
| Cerebellar Granular | 0.23 | 0.12 |
| layer | | |

DNA and RNA samples were subjected to barcode-seq analysis using the Illumina platform. For DNA barcode-seq, 1 μg total DNA was used for PCR amplification with primers indexed with sample-specific barcodes. For RNA barcode-seq, total RNA was treated with TURBO DNA-free Kit (Ambion: AM1907) to remove DNA, and reverse transcription was performed in 20 μl reaction volume with 1-2 μg RNA and gene-specific primer using RETROscript Kit (Ambion, AM1710), then PCR was performed with primers indexed with sample-specific barcodes. The PCR amplicons were mixed into a pool and subjected to Illumina sequencing for AAV Barcode-Seq (Adachi K et at., Nat Commun 5, 3075 (2014)). For the data analysis, first, the output data (barcode reads from tissues) was normalized to the input data (barcode reads from vector library). Second, the data of each AAV capsid in each brain region was normalized to that of the reference control, AAV9 (therefore, the value of AAV9 is always 1 in each sample). The relative values of distribution (Table 5 and 6) or transduction (Table 7 and 8) of each capsid compared with AAV9 are presented. In Tables 5-8, Occip Cortex, Hippoc. Cing gyrus, Thal, Hypoth. Cereb. Purknj., and Cereb. Granul. represent Occipital Cortex, Hippocampus, Cingulate gyrus, Thalamus, Hypothalamus. Cerebellar Purkinje, and Cerebellar Granular layer, respectively. Tables 5-8 show AAV vector distribution and transduction profiles of the 58 AAV capsids in different brain regions, as determined by DNA and RNA barcode-Seq analysis.

TABLE 5
AAV Capsid Distribution in Brain After CM Injection Based on DNA Barcode - Animal #1

| | Frontal | Occip. | | | | Cing. | | | | | Cereb | Cereb |
|---|---|---|---|---|---|---|---|---|---|---|---|---|
| | Gyrus | Cortex | Caudate | Putamen | Hippoc. | gyrus | Thal. | Hypoth | Pons | Medulla | Purknj | Granul |
| AAV1 | 24.2 | 21.4 | 43.2 | 3.8 | 14.8 | 14.7 | 37.8 | 17.0 | 19.1 | 21.3 | 24.4 | 39.5 |
| AAV1mt1 | 26.5 | 25.1 | 42.3 | 4.2 | 18.3 | 16.0 | 36.9 | 18.0 | 25.3 | 22.3 | 26.5 | 45.3 |
| AAV1mt2 | 0.2 | 0.1 | 0.6 | 0.5 | 0.1 | 0.1 | 0.2 | 0.1 | 0.1 | 0.1 | 0.1 | 0.3 |
| AAV1mt3 | 2.4 | 1.8 | 6.1 | 1.0 | 2.2 | 1.1 | 3.2 | 1.9 | 2.9 | 2.7 | 1.5 | 3.2 |
| AAV2 | 1.2 | 0.6 | 143.6 | 0.6 | 11.6 | 9.5 | 67.2 | 54.9 | 34.9 | 201.9 | 11.3 | 45.7 |
| AAV2mt1 | 0.7 | 0.9 | 0.7 | 1.1 | 1.0 | 1.0 | 0.9 | 0.9 | 0.5 | 0.6 | 0.7 | 1.0 |
| AAV2mt2 | 1.4 | 0.8 | 180.6 | 1.0 | 11.3 | 14.1 | 99.0 | 84.6 | 48.1 | 289.2 | 15.7 | 89.0 |
| AAV2mt3 | 0.1 | 0.0 | 22.0 | 0.3 | 1.5 | 0.6 | 9.2 | 8.8 | 2.8 | 111.3 | 1.7 | 4.0 |
| AAV2mt4 | 5.1 | 3.7 | 20.6 | 0.4 | 8.0 | 9.5 | 9.3 | 21.8 | 23.3 | 62.6 | 11.0 | 13.0 |
| AAV2mt5 | 8.7 | 4.9 | 27.1 | 1.3 | 23.4 | 45.2 | 17.4 | 80.5 | 86.2 | 245.4 | 31.9 | 30.1 |
| AAV2mt6 | 7.0 | 4.6 | 22.6 | 0.2 | 14.0 | 19.0 | 12.8 | 37.9 | 37.3 | 123.9 | 18.3 | 17.7 |
| AAV2mt7 | 9.2 | 5.3 | 28.9 | 0.6 | 24.1 | 37.3 | 13.9 | 65.4 | 98.1 | 234.4 | 36.1 | 27.0 |
| AAV2mt8 | 13.1 | 8.0 | 14.8 | 0.9 | 24.2 | 45.9 | 7.5 | 77.4 | 91.3 | 259.4 | 29.2 | 22.0 |
| AAV2mt9 | 0.7 | 0.4 | 42.3 | 0.8 | 6.6 | 6.8 | 22.7 | 24.7 | 12.2 | 111.6 | 8.5 | 16.3 |
| AAV2mt10 | 7.2 | 4.2 | 11.8 | 0.5 | 13.8 | 24.8 | 4.8 | 40.0 | 27.8 | 96.5 | 18.7 | 14.4 |
| AAV3B | 4.8 | 3.9 | 1.2 | 0.2 | 2.9 | 9.8 | 0.3 | 39.9 | 13.8 | 58.2 | 11.9 | 1.3 |
| AAV3mt1 | 1.1 | 0.4 | 0.3 | 0.0 | 0.6 | 2.7 | 0.0 | 24.4 | 9.5 | 35.2 | 0.8 | 0.2 |
| AAV3mt2 | 2.2 | 1.2 | 0.6 | 0.0 | 0.8 | 3.8 | 0.0 | 23.7 | 5.7 | 44.5 | 2.8 | 0.4 |
| AAV3mt3 | 2.4 | 0.9 | 0.7 | 0.0 | 0.9 | 4.0 | 0.0 | 28.7 | 10.1 | 37.8 | 0.8 | 0.4 |
| AAV3mt4 | 3.3 | 2.3 | 0.3 | 0.0 | 2.0 | 6.4 | 0.2 | 30.6 | 11.1 | 43.0 | 7.5 | 1.1 |
| AAV4 | 0.2 | 0.2 | 4.0 | 0.7 | 0.9 | 1.0 | 39.9 | 2.3 | 2.9 | 8.1 | 2.0 | 10.9 |
| AAV5 | 0.5 | 0.7 | 2.6 | 1.5 | 2.1 | 2.0 | 7.6 | 4.1 | 1.3 | 5.8 | 2.6 | 4.3 |
| AAV5mt1 | 0.3 | 0.1 | 0.0 | 0.2 | 0.2 | 0.2 | 0.1 | 0.2 | 0.4 | 0.5 | 0.1 | 0.1 |
| AAV5mt2 | 0.4 | 0.3 | 0.0 | 0.0 | 0.7 | 0.6 | 1.0 | 1.1 | 0.2 | 1.5 | 0.8 | 1.1 |
| AAV5mt3 | 0.2 | 0.1 | 1.9 | 0.0 | 0.5 | 0.4 | 10.1 | 1.4 | 1.3 | 1.7 | 1.0 | 5.7 |
| AAV5mt4 | 0.1 | 0.2 | 0.0 | 0.0 | 0.0 | 0.2 | 0.0 | 0.0 | 0.0 | 0.4 | 0.0 | 0.3 |
| AAV5mt5 | 0.1 | 0.1 | 1.7 | 0.0 | 0.3 | 0.3 | 0.6 | 0.3 | 0.0 | 0.5 | 0.3 | 0.6 |
| AAV6 | 1.3 | 1.6 | 107.7 | 0.7 | 15.9 | 0.9 | 82.9 | 9.2 | 1.7 | 1.8 | 2.4 | 81.5 |
| AAV6mt1 | 0.9 | 1.2 | 81.2 | 0.4 | 11.5 | 0.7 | 65.8 | 6.0 | 1.0 | 1.5 | 1.8 | 58.4 |
| AAV6mt2 | 21.5 | 20.6 | 43.7 | 3.3 | 15.5 | 14.6 | 36.6 | 15.5 | 12.1 | 15.0 | 23.4 | 34.6 |
| AAV6mt3 | 2.1 | 2.9 | 82.9 | 1.2 | 15.1 | 1.6 | 54.1 | 8.2 | 2.2 | 3.1 | 3.7 | 52.1 |
| AAV6mt4 | 14.5 | 11.1 | 25.0 | 1.8 | 9.1 | 9.2 | 21.0 | 9.2 | 14.0 | 14.7 | 15.3 | 26.6 |
| AAV6mt5 | 33.0 | 25.5 | 42.2 | 5.4 | 20.4 | 19.9 | 39.0 | 21.5 | 28.9 | 25.9 | 32.0 | 45.8 |
| AAV7 | 1.7 | 1.9 | 1.0 | 1.3 | 2.2 | 2.2 | 1.3 | 1.9 | 1.5 | 2.1 | 1.5 | 1.9 |
| AAV8 | 12.1 | 15.1 | 3.3 | 4.3 | 6.3 | 8.0 | 9.0 | 8.2 | 13.2 | 11.6 | 14.6 | 11.7 |
| AAV9 | 1.0 | 1.0 | 1.0 | 1.0 | 1.0 | 1.0 | 1.0 | 1.0 | 1.0 | 1.0 | 1.0 | 1.0 |
| AAV9mt1 | 5.7 | 3.4 | 24.8 | 0.4 | 11.8 | 17.1 | 12.5 | 34.0 | 35.2 | 111.7 | 17.1 | 16.6 |
| AAV9mt2 | 0.2 | 0.1 | 0.5 | 0.2 | 0.1 | 0.1 | 0.1 | 0.1 | 0.0 | 0.0 | 0.1 | 0.1 |
| AAV9mt3 | 0.4 | 0.5 | 0.7 | 0.7 | 0.7 | 0.6 | 0.5 | 0.5 | 0.3 | 0.2 | 0.4 | 0.7 |
| AAV9mt4 | 0.1 | 0.0 | 0.0 | 0.0 | 0.0 | 0.0 | 0.1 | 0.0 | 0.0 | 0.0 | 0.0 | 0.1 |
| AAV9mt5 | 0.5 | 0.5 | 1.6 | 1.8 | 0.5 | 0.6 | 0.3 | 0.3 | 0.0 | 0.1 | 0.3 | 0.3 |
| AAV9mt6 | 3.2 | 2.5 | 38.9 | 24.2 | 3.0 | 3.1 | 94.3 | 105.5 | 2.6 | 7.7 | 9.8 | 67.2 |
| AAV9mt7 | 2.5 | 3.0 | 0.9 | 1.3 | 2.6 | 2.6 | 2.6 | 3.0 | 2.3 | 2.0 | 2.5 | 2.2 |
| AAV9mt8 | 3.0 | 2.9 | 5.2 | 3.9 | 3.6 | 2.8 | 1.6 | 2.6 | 1.4 | 1.9 | 3.0 | 2.3 |
| AAV9mt9 | 0.1 | 0.1 | 0.0 | 0.0 | 0.0 | 0.1 | 0.0 | 0.0 | 0.0 | 0.0 | 0.0 | 0.0 |
| AAV9mt10 | 0.1 | 0.0 | 0.0 | 0.0 | 0.0 | 0.0 | 0.0 | 0.0 | 0.0 | 0.1 | 0.0 | 0.0 |
| AAV9mt11 | 1.5 | 1.7 | 1.3 | 1.8 | 1.5 | 1.6 | 1.6 | 1.8 | 1.0 | 1.4 | 1.9 | 1.8 |
| AAV9mt12 | 0.1 | 0.0 | 0.0 | 0.0 | 0.0 | 0.1 | 0.0 | 0.0 | 0.0 | 0.0 | 0.0 | 0.1 |
| AAV11 | 27.8 | 27.7 | 1.7 | 7.0 | 66.3 | 64.9 | 0.5 | 75.0 | 25.4 | 61.1 | 52.2 | 20.4 |
| AAVrh8 | 2.6 | 3.6 | 2.7 | 2.1 | 2.9 | 2.8 | 3.2 | 2.6 | 1.9 | 1.9 | 2.8 | 3.0 |
| AAVrh10 | 14.6 | 15.8 | 2.6 | 3.5 | 6.1 | 7.2 | 6.3 | 7.2 | 14.6 | 18.3 | 11.5 | 10.1 |
| AAVrh39 | 21.9 | 21.9 | 4.6 | 3.8 | 8.1 | 10.9 | 10.2 | 10.3 | 20.6 | 26.2 | 16.2 | 16.0 |
| AAVrh43 | 6.2 | 7.2 | 1.8 | 2.0 | 3.5 | 4.0 | 4.9 | 3.9 | 6.0 | 5.0 | 8.4 | 6.0 |
| AAVDJ | 22.2 | 14.5 | 17.0 | 1.6 | 40.3 | 51.8 | 10.4 | 113.9 | 142.9 | 335.1 | 44.5 | 28.2 |
| AAVDJ8 | 7.0 | 6.9 | 3.3 | 2.8 | 4.4 | 4.4 | 5.1 | 3.9 | 5.6 | 5.5 | 6.6 | 7.1 |
| Pig | 10.3 | 7.8 | 2.8 | 2.7 | 6.2 | 6.9 | 5.1 | 5.9 | 14.4 | 13.1 | 8.5 | 9.6 |
| Mouse | 0.1 | 0.1 | 0.0 | 0.2 | 0.0 | 0.1 | 0.0 | 0.1 | 0.0 | 0.0 | 0.0 | 0.0 |
| Avian | 0.0 | 0.0 | 1.9 | 0.0 | 0.0 | 0.1 | 0.0 | 0.0 | 0.0 | 0.1 | 0.0 | 0.1 |

TABLE 6
AAV Capsid Distribution in Brain After CM Injection Based on DNA Barcode - Animal #2

| | Frontal | Occip. | | | | Cing. | | | | | Cereb. | Cereb. |
|---|---|---|---|---|---|---|---|---|---|---|---|---|
| | Gyrus | Cortex | Caudate | Putamen | Hippoc | gyrus | Thal. | Hypoth | Pons | Medulla | Purknj | Granul |
| AAV1 | 13.4 | 16.8 | 62.2 | 1.8 | 17.4 | 17.5 | 9.5 | 14.1 | 13.4 | 10.6 | 19.1 | 19.4 |
| AAV1mt1 | 15.2 | 18.6 | 76.9 | 3.1 | 20.1 | 21.8 | 10.3 | 16.8 | 14.7 | 12.5 | 22.2 | 22.3 |
| AAV1mt2 | 0.4 | 0.1 | 0.7 | 0.1 | 0.2 | 0.4 | 0.0 | 0.3 | 0.1 | 0.3 | 0.0 | 0.1 |
| AAV1mt3 | 1.5 | 1.4 | 5.2 | 0.9 | 2.2 | 1.9 | 3.5 | 1.9 | 2.4 | 1.2 | 1.5 | 1.6 |
| AAV2 | 3.3 | 0.8 | 576.1 | 0.4 | 56.3 | 12.3 | 13.1 | 223.5 | 3.6 | 66.1 | 9.4 | 15.4 |
| AAV2mt1 | 1.3 | 0.8 | 1.6 | 1.6 | 1.4 | 0.7 | 1.8 | 1.1 | 1.1 | 1.4 | 0.5 | 1.2 |
| AAV2mt2 | 2.3 | 1.0 | 570.1 | 0.6 | 53.4 | 18.9 | 18.9 | 269.0 | 12.2 | 115.5 | 11.0 | 21.7 |
| AAV2mt3 | 1.0 | 0.0 | 70.9 | 0.0 | 9.7 | 1.0 | 1.2 | 25.2 | 0.0 | 30.7 | 1.8 | 1.7 |
| AAV2mt4 | 4.5 | 3.7 | 70.1 | 0.3 | 8.4 | 13.9 | 1.6 | 35.6 | 11.6 | 26.2 | 7.5 | 5.6 |
| AAV2mt5 | 8.6 | 7.4 | 158.7 | 0.5 | 25.5 | 54.9 | 5.1 | 101.2 | 29.2 | 126.4 | 28.6 | 14.7 |
| AAV2mt6 | 7.1 | 4.9 | 93.3 | 0.3 | 12.3 | 28.9 | 2.3 | 45.4 | 18.3 | 52.0 | 14.1 | 8.2 |
| AAV2mt7 | 10.5 | 5.2 | 135.3 | 0.1 | 17.7 | 53.2 | 4.8 | 69.0 | 33.1 | 99.2 | 27.9 | 13.6 |
| AAV2mt8 | 14.9 | 10.2 | 101.6 | 0.6 | 16.1 | 72.0 | 4.3 | 65.8 | 37.4 | 109.5 | 26.5 | 13.7 |
| AAV2mt9 | 2.3 | 0.7 | 203.9 | 0.1 | 23.9 | 8.3 | 5.3 | 78.5 | 1.6 | 50.7 | 8.0 | 7.1 |
| AAV2mt10 | 7.9 | 7.7 | 63.8 | 0.4 | 12.4 | 42.6 | 1.5 | 41.7 | 14.0 | 63.3 | 19.2 | 11.5 |
| AAV3B | 8.2 | 3.4 | 2.9 | 0.2 | 5.9 | 36.9 | 1.4 | 16.8 | 7.4 | 29.3 | 8.9 | 1.4 |
| AAV3mt1 | 5.2 | 1.3 | 0.1 | 0.0 | 3.2 | 32.9 | 0.1 | 16.8 | 5.5 | 30.4 | 1.9 | 0.6 |
| AAV3mt2 | 5.6 | 1.9 | 0.8 | 0.1 | 4.6 | 25.7 | 0.3 | 11.1 | 3.5 | 27.1 | 3.0 | 0.9 |
| AAV3mt3 | 6.1 | 1.6 | 0.0 | 0.0 | 4.1 | 32.5 | 0.2 | 12.5 | 6.1 | 24.6 | 1.5 | 0.7 |
| AAV3mt4 | 5.8 | 2.3 | 1.4 | 0.0 | 4.3 | 29.7 | 0.6 | 12.9 | 7.9 | 22.4 | 5.6 | 1.0 |
| AAV4 | 0.4 | 0.2 | 2.0 | 0.5 | 9.9 | 0.9 | 19.4 | 4.3 | 3.6 | 4.3 | 0.5 | 1.0 |
| AAV5 | 3.1 | 1.3 | 17.9 | 0.6 | 8.2 | 3.0 | 10.8 | 8.5 | 1.3 | 6.7 | 2.5 | 4.0 |
| AAV5mt1 | 1.3 | 0.9 | 0.5 | 0.0 | 0.3 | 0.2 | 0.1 | 1.8 | 0.0 | 1.1 | 0.1 | 0.1 |
| AAV5mt2 | 1.3 | 1.1 | 3.6 | 0.2 | 2.1 | 1.2 | 0.8 | 2.0 | 0.4 | 1.0 | 1.1 | 1.5 |
| AAV5mt3 | 0.2 | 0.5 | 17.3 | 0.0 | 6.1 | 1.0 | 6.0 | 11.9 | 0.2 | 1.3 | 0.5 | 4.2 |
| AAV5mt4 | 0.6 | 0.6 | 0.4 | 0.0 | 0.2 | 0.1 | 0.0 | 0.6 | 0.0 | 0.2 | 0.1 | 0.1 |
| AAV5mt5 | 0.8 | 0.5 | 5.3 | 0.0 | 0.7 | 0.1 | 0.0 | 2.9 | 0.0 | 0.6 | 0.6 | 0.5 |
| AAV6 | 1.6 | 0.9 | 203.6 | 0.1 | 45.4 | 1.5 | 39.5 | 6.4 | 0.2 | 2.1 | 1.3 | 6.3 |
| AAV6mt1 | 0.7 | 0.8 | 151.1 | 0.0 | 33.8 | 1.3 | 25.2 | 6.1 | 0.5 | 0.8 | 1.0 | 4.8 |
| AAV6mt2 | 11.2 | 16.5 | 67.2 | 1.9 | 17.4 | 17.5 | 9.6 | 14.0 | 8.7 | 8.1 | 19.5 | 19.1 |
| AAV6mt3 | 1.2 | 1.7 | 176.0 | 0.1 | 32.8 | 2.4 | 21.9 | 6.1 | 1.8 | 1.2 | 2.4 | 5.3 |
| AAV6mt4 | 6.6 | 11.2 | 31.0 | 1.1 | 9.3 | 12.8 | 7.7 | 9.9 | 9.7 | 7.9 | 12.2 | 12.0 |
| AAV6mt5 | 16.4 | 22.7 | 65.5 | 2.9 | 20.1 | 27.7 | 11.3 | 20.4 | 12.7 | 14.7 | 27.1 | 24.1 |
| AAV7 | 3.9 | 1.5 | 0.4 | 0.4 | 1.0 | 3.6 | 1.0 | 1.0 | 0.9 | 1.5 | 1.0 | 0.9 |
| AAV8 | 10.8 | 13.4 | 7.1 | 4.8 | 6.6 | 10.3 | 9.1 | 6.2 | 7.6 | 8.7 | 14.2 | 12.7 |
| AAV9 | 1.0 | 1.0 | 1.0 | 1.0 | 1.0 | 1.0 | 1.0 | 1.0 | 1.0 | 1.0 | 1.0 | 1.0 |
| AAV9mt1 | 5.6 | 4.4 | 85.8 | 0.5 | 12.3 | 23.8 | 1.6 | 41.8 | 16.3 | 48.7 | 12.3 | 7.5 |
| AAV9mt2 | 0.3 | 0.1 | 0.1 | 0.3 | 0.1 | 0.2 | 0.4 | 0.2 | 0.1 | 0.1 | 0.0 | 0.1 |
| AAV9mt3 | 0.3 | 0.4 | 0.5 | 1.0 | 0.8 | 0.4 | 1.0 | 0.5 | 0.4 | 0.2 | 0.3 | 0.8 |
| AAV9mt4 | 0.2 | 0.0 | 0.0 | 0.0 | 0.1 | 0.2 | 0.0 | 0.8 | 0.0 | 0.0 | 0.0 | 0.2 |
| AAV9mt5 | 0.4 | 0.4 | 1.0 | 1.1 | 0.5 | 0.5 | 1.4 | 0.4 | 0.2 | 0.0 | 0.2 | 0.6 |
| AAV9mt6 | 1.4 | 1.1 | 51.3 | 20.5 | 36.8 | 3.0 | 97.8 | 673.2 | 2.6 | 4.5 | 1.4 | 33.3 |
| AAV9mt7 | 1.7 | 3.0 | 4.6 | 3.4 | 2.9 | 2.9 | 0.0 | 2.6 | 1.9 | 1.2 | 2.7 | 2.6 |
| AAV9mt8 | 1.9 | 3.8 | 4.3 | 2.2 | 3.0 | 2.7 | 5.6 | 2.1 | 3.6 | 1.5 | 3.3 | 3.4 |
| AAV9mt9 | 0.1 | 0.0 | 0.0 | 0.0 | 0.0 | 0.2 | 0.0 | 0.6 | 0.0 | 0.0 | 0.0 | 0.0 |
| AAV9mt10 | 0.1 | 0.0 | 0.0 | 0.2 | 0.0 | 0.1 | 0.4 | 0.1 | 0.0 | 0.0 | 0.0 | 0.0 |
| AAV9mt11 | 1.0 | 1.6 | 1.6 | 1.4 | 2.0 | 1.7 | 3.1 | 1.5 | 1.3 | 0.6 | 1.7 | 1.5 |
| AAV9mt12 | 0.2 | 0.0 | 0.0 | 0.1 | 0.0 | 0.2 | 0.0 | 0.1 | 0.0 | 0.0 | 0.0 | 0.1 |
| AAV11 | 19.9 | 54.8 | 2.7 | 1.1 | 15.6 | 220.3 | 6.5 | 60.4 | 22.6 | 47.2 | 62.1 | 19.3 |
| AAVrh8 | 2.2 | 3.2 | 5.2 | 3.7 | 3.5 | 2.8 | 5.6 | 1.8 | 2.3 | 1.1 | 3.0 | 4.2 |
| AAVrh10 | 12.4 | 15.2 | 7.1 | 5.7 | 6.1 | 9.5 | 8.9 | 5.1 | 7.2 | 7.2 | 10.7 | 10.0 |
| AAVrh39 | 18.6 | 21.6 | 9.5 | 6.0 | 7.9 | 16.1 | 9.5 | 8.0 | 11.1 | 11.9 | 17.7 | 15.9 |
| AAVrh43 | 4.2 | 7.1 | 3.8 | 2.7 | 3.6 | 5.2 | 4.4 | 3.1 | 4.0 | 3.3 | 6.9 | 7.1 |
| AAVDJ | 19.6 | 13.8 | 80.6 | 1.0 | 18.6 | 91.9 | 5.6 | 94.7 | 55.3 | 139.9 | 32.5 | 15.6 |
| AAVDJ8 | 5.7 | 6.3 | 5.9 | 3.1 | 4.0 | 7.6 | 5.7 | 3.7 | 3.9 | 4.7 | 5.9 | 8.5 |
| Pig | 9.5 | 8.3 | 4.7 | 3.7 | 4.4 | 14.7 | 6.2 | 5.1 | 9.9 | 9.0 | 9.4 | 11.4 |
| Mouse | 0.5 | 0.2 | 0.0 | 0.1 | 0.0 | 0.3 | 0.0 | 0.0 | 0.0 | 0.1 | 0.0 | 0.0 |
| Avian | 0.3 | 0.0 | 0.0 | 0.0 | 0.0 | 0.0 | 0.0 | 0.3 | 0.0 | 0.2 | 0.0 | 0.0 |

TABLE 7
AAV Capsid Distribution in Brain After CM Injection Based on RNA Barcode - Animal #1

| | Frontal | Occip. | | | | Cing. | | | | | Cereb. | Cereb. |
|---|---|---|---|---|---|---|---|---|---|---|---|---|
| | Gyrus | Cortex | Caudate | Putamen | Hippoc | gyrus | Thal. | Hypoth | Pons | Medulla | Purknj | Granul |
| AAV1 | 1.4 | 3.4 | 4.0 | 0.2 | 3.0 | 1.3 | 6.9 | 6.5 | 14.4 | 8.0 | 5.4 | 4.0 |
| AAV1mt1 | 0.6 | 1.4 | 6.3 | 0.3 | 2.5 | 0.7 | 4.3 | 2.6 | 3.4 | 3.2 | 3.8 | 3.2 |
| AAV1mt2 | 0.0 | 0.0 | 0.9 | 0.0 | 0.0 | 0.0 | 0.0 | 0.0 | 0.0 | 0.0 | 0.0 | 0.0 |
| AAV1mt3 | 0.3 | 0.3 | 1.3 | 0.2 | 0.7 | 0.3 | 1.4 | 1.0 | 0.3 | 0.2 | 0.3 | 0.5 |
| AAV2 | 0.3 | 0.3 | 5.1 | 0.3 | 2.5 | 0.5 | 12.4 | 2.5 | 0.9 | 1.0 | 0.3 | 6.2 |
| AAV2mt1 | 1.5 | 1.5 | 1.6 | 1.7 | 1.3 | 1.5 | 1.6 | 0.9 | 0.8 | 0.2 | 0.5 | 0.9 |
| AAV2mt2 | 0.1 | 0.1 | 3.3 | 0.1 | 1.4 | 0.4 | 24.1 | 3.6 | 0.4 | 3.6 | 0.3 | 10.3 |
| AAV2mt3 | 0.0 | 0.1 | 1.6 | 0.0 | 1.1 | 0.0 | 1.5 | 0.1 | 0.2 | 0.0 | 0.1 | 1.1 |
| AAV2mt4 | 0.5 | 1.2 | 0.6 | 0.1 | 1.2 | 0.4 | 2.0 | 1.6 | 3.4 | 3.1 | 1.9 | 1.6 |
| AAV2mt5 | 0.8 | 1.3 | 0.5 | 0.5 | 1.3 | 0.9 | 3.9 | 2.3 | 11.2 | 8.1 | 3.9 | 2.7 |
| AAV2mt6 | 1.0 | 1.4 | 1.3 | 0.1 | 0.7 | 0.5 | 2.0 | 1.4 | 4.7 | 1.7 | 1.7 | 1.2 |
| AAV2mt7 | 0.5 | 1.3 | 2.0 | 0.4 | 1.1 | 0.8 | 3.7 | 2.7 | 8.5 | 10.4 | 4.9 | 1.8 |
| AAV2mt8 | 0.9 | 1.6 | 3.2 | 0.6 | 0.8 | 0.9 | 1.4 | 1.6 | 11.8 | 10.7 | 3.1 | 1.1 |
| AAV2mt9 | 0.2 | 0.1 | 2.2 | 0.0 | 1.3 | 0.2 | 4.3 | 1.2 | 0.5 | 0.4 | 0.6 | 2.5 |
| AAV2mt10 | 0.5 | 0.7 | 1.4 | 0.0 | 0.6 | 0.5 | 1.1 | 1.6 | 9.5 | 6.1 | 1.6 | 1.2 |
| AAV3B | 5.2 | 12.0 | 0.5 | 0.1 | 0.4 | 2.6 | 0.2 | 5.7 | 70.0 | 91.8 | 24.8 | 0.3 |
| AAV3mt1 | 0.0 | 0.0 | 0.0 | 0.0 | 0.0 | 0.0 | 0.0 | 0.0 | 0.0 | 0.0 | 0.0 | 0.0 |
| AAV3mt2 | 1.1 | 1.2 | 0.0 | 0.0 | 0.1 | 0.3 | 0.0 | 0.8 | 6.7 | 6.7 | 3.8 | 0.0 |
| AAV3mt3 | 0.0 | 0.0 | 0.0 | 0.0 | 0.0 | 0.0 | 0.0 | 0.0 | 0.0 | 0.0 | 0.0 | 0.0 |
| AAV3mt4 | 3.2 | 6.1 | 0.0 | 0.0 | 0.1 | 1.8 | 0.1 | 4.4 | 60.4 | 63.6 | 14.7 | 0.1 |
| AAV4 | 0.0 | 0.0 | 0.7 | 0.0 | 0.3 | 0.1 | 6.0 | 0.1 | 0.0 | 0.0 | 0.1 | 0.3 |
| AAV5 | 0.0 | 0.1 | 1.0 | 0.2 | 0.3 | 0.1 | 1.7 | 0.3 | 0.4 | 0.6 | 0.3 | 0.2 |
| AAV5mt1 | 0.0 | 0.0 | 0.0 | 0.0 | 0.1 | 0.0 | 0.2 | 0.0 | 0.0 | 0.1 | 0.0 | 0.0 |
| AAV5mt2 | 0.1 | 0.0 | 5.1 | 0.0 | 0.1 | 0.0 | 0.1 | 0.1 | 0.1 | 0.0 | 0.1 | 0.0 |
| AAV5mt3 | 0.0 | 0.0 | 0.0 | 0.0 | 0.2 | 0.0 | 3.3 | 0.2 | 0.0 | 0.0 | 0.0 | 0.8 |
| AAV5mt4 | 0.0 | 0.0 | 0.0 | 0.0 | 0.1 | 0.0 | 0.0 | 0.0 | 0.0 | 0.0 | 0.0 | 0.0 |
| AAV5mt5 | 0.0 | 0.0 | 0.0 | 0.0 | 0.0 | 0.0 | 0.0 | 0.0 | 0.1 | 0.0 | 0.1 | 0.0 |
| AAV6 | 0.2 | 0.8 | 27.5 | 0.0 | 20.6 | 0.4 | 48.5 | 6.7 | 3.1 | 0.2 | 1.7 | 23.9 |
| AAV6mt1 | 0.3 | 0.8 | 22.1 | 0.1 | 27.6 | 0.4 | 62.7 | 10.1 | 3.7 | 1.1 | 1.6 | 33.5 |
| AAV6mt2 | 0.6 | 1.7 | 4.3 | 0.8 | 2.1 | 0.7 | 4.7 | 3.2 | 5.4 | 3.6 | 2.1 | 2.5 |
| AAV6mt3 | 0.6 | 2.0 | 7.8 | 0.0 | 24.2 | 0.5 | 22.5 | 4.8 | 8.7 | 2.1 | 3.0 | 18.3 |
| AAV6mt4 | 1.4 | 2.4 | 17.1 | 0.0 | 2.3 | 0.8 | 2.6 | 4.0 | 3.0 | 6.4 | 2.6 | 2.4 |
| AAV6mt5 | 1.0 | 3.5 | 2.0 | 0.1 | 3.0 | 1.4 | 6.5 | 5.0 | 5.7 | 7.6 | 6.2 | 4.3 |
| AAV7 | 4.8 | 4.8 | 4.4 | 4.0 | 5.0 | 5.6 | 3.9 | 3.8 | 1.6 | 2.4 | 3.1 | 4.1 |
| AAV8 | 4.1 | 4.8 | 0.8 | 2.6 | 2.0 | 3.8 | 2.6 | 5.1 | 10.9 | 13.1 | 7.6 | 3.6 |
| AAV9 | 1.0 | 1.0 | 1.0 | 1.0 | 1.0 | 1.0 | 1.0 | 1.0 | 1.0 | 1.0 | 1.0 | 1.0 |
| AAV9mt1 | 0.5 | 1.3 | 2.9 | 0.2 | 0.7 | 0.5 | 2.8 | 1.3 | 4.7 | 3.9 | 2.3 | 1.7 |
| AAV9mt2 | 0.3 | 0.2 | 0.0 | 0.5 | 0.3 | 0.4 | 0.4 | 0.1 | 0.0 | 0.0 | 0.1 | 0.1 |
| AAV9mt3 | 1.0 | 0.7 | 0.4 | 0.5 | 0.7 | 0.9 | 0.6 | 0.5 | 0.9 | 0.3 | 0.7 | 0.9 |
| AAV9mt4 | 0.0 | 0.0 | 0.0 | 0.0 | 0.3 | 0.0 | 0.0 | 0.0 | 0.0 | 0.0 | 0.3 | 0.0 |
| AAV9mt5 | 3.3 | 3.0 | 1.9 | 2.3 | 2.6 | 3.0 | 5.2 | 0.9 | 0.2 | 0.0 | 0.8 | 1.1 |
| AAV9mt6 | 0.2 | 0.8 | 23.6 | 0.0 | 0.1 | 0.1 | 8.3 | 2.3 | 0.4 | 0.2 | 2.2 | 12.8 |
| AAV9mt7 | 0.9 | 1.5 | 0.0 | 0.2 | 1.1 | 2.0 | 0.6 | 1.0 | 0.7 | 10.8 | 0.7 | 1.3 |
| AAV9mt8 | 1.9 | 1.8 | 0.0 | 3.7 | 0.7 | 1.3 | 1.4 | 0.8 | 1.3 | 3.2 | 1.1 | 0.6 |
| AAV9mt9 | 0.0 | 0.0 | 0.0 | 0.4 | 0.1 | 0.0 | 0.0 | 0.0 | 0.0 | 0.0 | 0.0 | 0.0 |
| AAV9mt10 | 0.0 | 0.0 | 0.0 | 0.0 | 0.0 | 0.0 | 0.0 | 0.0 | 0.0 | 0.0 | 0.0 | 0.0 |
| AAV9mt11 | 1.9 | 1.4 | 0.9 | 1.8 | 1.4 | 1.8 | 1.6 | 2.0 | 1.8 | 0.7 | 1.6 | 1.6 |
| AAV9mt12 | 0.0 | 0.0 | 0.0 | 0.0 | 0.0 | 0.0 | 0.0 | 0.0 | 0.0 | 0.0 | 0.0 | 0.0 |
| AAV11 | 0.2 | 0.3 | 0.0 | 0.0 | 0.4 | 0.5 | 0.2 | 1.5 | 0.4 | 1.9 | 0.8 | 0.0 |
| AAVrh8 | 2.4 | 2.0 | 1.5 | 3.0 | 2.0 | 2.4 | 1.0 | 1.7 | 0.7 | 0.9 | 1.7 | 1.4 |
| AAVrh10 | 2.2 | 4.0 | 1.5 | 0.7 | 1.6 | 1.8 | 1.0 | 2.0 | 3.7 | 4.4 | 3.4 | 1.5 |
| AAVrh39 | 2.8 | 6.5 | 1.9 | 1.1 | 1.5 | 2.1 | 0.9 | 2.8 | 5.3 | 6.6 | 3.7 | 1.5 |
| AAVrh43 | 1.7 | 3.0 | 3.1 | 0.6 | 1.3 | 1.8 | 1.0 | 2.6 | 5.4 | 4.0 | 3.3 | 1.7 |
| AAVDJ | 2.2 | 3.3 | 1.7 | 0.1 | 1.1 | 2.2 | 3.5 | 3.9 | 10.6 | 13.1 | 5.5 | 2.6 |
| AAVDJ8 | 0.5 | 0.9 | 0.8 | 0.1 | 0.5 | 0.7 | 0.9 | 1.2 | 0.6 | 1.1 | 0.9 | 0.4 |
| Pig | 0.1 | 0.3 | 0.4 | 0.0 | 0.3 | 0.2 | 0.3 | 0.3 | 0.8 | 1.0 | 0.4 | 0.2 |
| Mouse | 0.0 | 0.0 | 0.0 | 0.1 | 0.1 | 0.0 | 0.0 | 0.0 | 0.0 | 0.0 | 0.0 | 0.0 |
| Avian | 0.0 | 0.0 | 0.0 | 0.0 | 0.0 | 0.0 | 0.0 | 0.0 | 0.0 | 0.0 | 0.0 | 0.0 |

TABLE 8
AAV Capsid Distribution in Brain After CM Injection Based on RNA Barcode - Animal #2

| | Frontal | Occip. | | | | Cing | | | | | Cereb. | Cereb. |
|---|---|---|---|---|---|---|---|---|---|---|---|---|
| | Gyrus | Cortex | Caudate | Putamen | Hippoc | gyrus | Thal | Hypoth | Pons | Medulla | Purknj | Granul |
| AAV1 | 0.6 | 1.1 | 9.1 | 0.2 | 3.2 | 3.7 | 16.5 | 8.1 | 2.3 | 1.1 | 3.5 | 1.2 |
| AAV1mt1 | 0.5 | 0.6 | 7.9 | 0.2 | 1.8 | 1.9 | 5.4 | 3.8 | 2.7 | 1.3 | 1.7 | 0.9 |
| AAV1mt2 | 0.0 | 0.0 | 0.0 | 0.0 | 0.0 | 0.0 | 0.0 | 0.0 | 0.0 | 0.0 | 0.0 | 0.0 |
| AAV1mt3 | 0.1 | 0.2 | 0.1 | 0.0 | 0.6 | 0.7 | 0.6 | 2.6 | 0.7 | 0.1 | 0.6 | 0.2 |
| AAV2 | 0.1 | 0.4 | 33.5 | 0.0 | 3.2 | 0.4 | 15.0 | 32.8 | 0.2 | 0.4 | 0.2 | 1.5 |
| AAV2mt1 | 1.2 | 1.4 | 1.3 | 1.1 | 0.9 | 1.1 | 1.5 | 0.3 | 1.1 | 0.3 | 0.6 | 1.0 |
| AAV2mt2 | 0.1 | 0.1 | 65.3 | 0.0 | 1.6 | 0.2 | 12.8 | 50.8 | 0.1 | 0.2 | 0.1 | 1.4 |
| AAV2mt3 | 0.1 | 0.1 | 10.7 | 0.0 | 1.4 | 0.1 | 2.2 | 1.1 | 0.0 | 0.0 | 0.0 | 0.2 |
| AAV2mt4 | 0.9 | 0.5 | 10.8 | 0.0 | 1.1 | 1.3 | 2.7 | 11.1 | 0.2 | 0.3 | 0.5 | 0.5 |
| AAV2mt5 | 1.2 | 0.3 | 13.8 | 0.0 | 1.2 | 3.8 | 8.2 | 24.7 | 0.5 | 1.3 | 2.4 | 1.1 |
| AAV2mt6 | 0.3 | 0.1 | 7.2 | 0.0 | 0.9 | 0.8 | 4.5 | 13.6 | 1.0 | 0.6 | 1.2 | 0.4 |
| AAV2mt7 | 0.3 | 0.2 | 9.6 | 0.1 | 1.1 | 1.2 | 3.9 | 16.4 | 1.0 | 0.6 | 2.7 | 0.6 |
| AAV2mt8 | 0.8 | 0.5 | 5.0 | 0.1 | 1.1 | 4.6 | 2.0 | 11.3 | 0.6 | 1.1 | 2.8 | 0.5 |
| AAV2mt9 | 0.0 | 0.1 | 14.6 | 0.1 | 1.5 | 1.6 | 4.5 | 21.3 | 0.1 | 0.3 | 0.5 | 0.6 |
| AAV2mt10 | 1.5 | 0.1 | 21.6 | 0.0 | 0.9 | 1.4 | 2.8 | 11.6 | 0.0 | 0.4 | 1.0 | 0.6 |
| AAV3B | 2.6 | 0.8 | 0.3 | 1.1 | 1.2 | 9.4 | 0.2 | 2.7 | 13.0 | 13.6 | 14.3 | 0.2 |
| AAV3mt1 | 0.0 | 0.0 | 0.0 | 0.0 | 0.0 | 0.0 | 0.0 | 0.0 | 0.0 | 0.0 | 0.0 | 0.0 |
| AAV3mt2 | 0.3 | 0.2 | 0.0 | 0.1 | 0.1 | 0.5 | 0.0 | 0.2 | 0.7 | 1.0 | 2.3 | 0.0 |
| AAV3mt3 | 0.0 | 0.0 | 0.0 | 0.0 | 0.0 | 0.0 | 0.0 | 0.0 | 0.0 | 0.2 | 0.0 | 0.0 |
| AAV3mt4 | 2.2 | 0.6 | 1.7 | 0.0 | 0.6 | 4.6 | 0.3 | 2.0 | 10.8 | 13.3 | 10.1 | 0.0 |
| AAV4 | 0.0 | 0.0 | 0.1 | 0.0 | 0.8 | 0.1 | 13.2 | 0.4 | 0.0 | 0.0 | 0.0 | 0.0 |
| AAV5 | 0.0 | 0.0 | 3.1 | 0.2 | 0.9 | 0.3 | 7.7 | 1.5 | 0.0 | 0.1 | 0.2 | 0.3 |
| AAV5mt1 | 0.0 | 0.0 | 0.0 | 0.0 | 0.0 | 0.0 | 0.0 | 0.0 | 0.0 | 0.0 | 0.0 | 0.0 |
| AAV5mt2 | 0.1 | 0.0 | 0.0 | 0.0 | 0.1 | 0.0 | 0.9 | 0.2 | 0.0 | 0.0 | 0.5 | 0.0 |
| AAV5mt3 | 0.0 | 0.0 | 2.4 | 0.0 | 0.4 | 0.0 | 8.5 | 2.0 | 0.0 | 0.0 | 0.1 | 0.5 |
| AAV5mt4 | 0.0 | 0.0 | 0.0 | 0.0 | 0.0 | 0.0 | 0.0 | 0.0 | 0.0 | 0.0 | 0.0 | 0.0 |
| AAV5mt5 | 0.0 | 0.0 | 0.0 | 0.0 | 0.0 | 0.0 | 0.4 | 0.0 | 0.0 | 0.0 | 0.0 | 0.0 |
| AAV6 | 0.0 | 0.2 | 60.0 | 0.2 | 11.2 | 0.2 | 95.8 | 9.5 | 0.1 | 0.0 | 0.5 | 2.2 |
| AAV6mt1 | 0.9 | 0.1 | 70.7 | 0.1 | 15.5 | 1.4 | 156.4 | 9.0 | 0.1 | 0.3 | 0.5 | 2.5 |
| AAV6mt2 | 0.3 | 0.4 | 10.3 | 0.3 | 1.5 | 1.2 | 5.6 | 6.3 | 1.3 | 1.0 | 1.2 | 0.7 |
| AAV6mt3 | 0.0 | 0.1 | 22.6 | 0.0 | 8.8 | 1.6 | 42.4 | 11.3 | 0.7 | 0.3 | 0.9 | 1.5 |
| AAV6mt4 | 0.5 | 0.7 | 4.7 | 0.0 | 1.4 | 2.4 | 8.5 | 6.9 | 0.9 | 0.3 | 1.5 | 0.7 |
| AAV6mt5 | 1.2 | 1.0 | 18.8 | 0.0 | 5.5 | 4.7 | 15.9 | 11.5 | 0.9 | 0.9 | 5.3 | 1.2 |
| AAV7 | 1.4 | 1.1 | 2.6 | 0.3 | 0.9 | 0.9 | 0.1 | 0.2 | 0.2 | 0.3 | 1.0 | 0.2 |
| AAV8 | 3.9 | 3.7 | 1.8 | 1.5 | 1.9 | 5.6 | 2.6 | 5.2 | 1.9 | 5.8 | 5.0 | 3.6 |
| AAV9 | 1.0 | 1.0 | 1.0 | 1.0 | 1.0 | 1.0 | 1.0 | 1.0 | 1.0 | 1.0 | 1.0 | 1.0 |
| AAV9mt1 | 0.4 | 0.6 | 5.3 | 0.0 | 0.7 | 0.7 | 4.2 | 8.9 | 0.2 | 0.5 | 0.8 | 0.7 |
| AAV9mt2 | 0.6 | 0.3 | 0.4 | 0.1 | 0.2 | 0.1 | 0.5 | 0.3 | 1.2 | 0.0 | 0.0 | 0.1 |
| AAV9mt3 | 0.9 | 1.1 | 1.4 | 0.5 | 0.6 | 0.5 | 0.9 | 0.3 | 0.7 | 1.1 | 0.5 | 0.7 |
| AAV9mt4 | 0.0 | 0.0 | 0.0 | 0.0 | 0.0 | 0.0 | 0.0 | 0.0 | 0.0 | 0.0 | 0.0 | 0.0 |
| AAV9mt5 | 5.7 | 4.1 | 1.7 | 1.5 | 2.7 | 1.7 | 5.0 | 1.2 | 1.4 | 0.8 | 1.5 | 1.3 |
| AAV9mt6 | 0.0 | 0.0 | 0.1 | 0.0 | 2.4 | 4.7 | 49.1 | 98.5 | 0.0 | 0.0 | 0.2 | 3.1 |
| AAV9mt7 | 0.8 | 1.6 | 2.9 | 0.6 | 0.7 | 0.6 | 0.1 | 1.4 | 5.8 | 3.5 | 0.3 | 1.1 |
| AAV9mt8 | 3.7 | 0.8 | 0.0 | 0.0 | 1.3 | 0.5 | 0.2 | 0.5 | 0.1 | 0.0 | 1.0 | 3.3 |
| AAV9mt9 | 0.0 | 0.0 | 0.0 | 0.0 | 0.0 | 0.3 | 0.0 | 0.0 | 0.0 | 0.0 | 0.0 | 0.0 |
| AAV9mt10 | 0.0 | 0.0 | 0.0 | 0.0 | 0.0 | 0.0 | 0.0 | 0.0 | 0.0 | 0.0 | 0.0 | 0.0 |
| AAV9mt11 | 1.6 | 1.9 | 5.7 | 0.1 | 2.2 | 2.4 | 0.7 | 0.9 | 4.0 | 3.7 | 1.6 | 1.6 |
| AAV9mt12 | 0.0 | 0.0 | 0.0 | 0.0 | 0.0 | 0.0 | 0.0 | 0.0 | 0.0 | 0.0 | 0.0 | 0.0 |
| AAV11 | 0.1 | 0.1 | 0.6 | 0.2 | 0.1 | 5.6 | 0.0 | 0.4 | 1.1 | 0.0 | 1.5 | 0.0 |
| AAVrh8 | 1.5 | 2.3 | 7.4 | 0.8 | 2.2 | 2.1 | 1.8 | 1.1 | 2.1 | 1.1 | 1.5 | 1.3 |
| AAVrh10 | 2.0 | 2.4 | 2.3 | 0.7 | 1.5 | 1.7 | 0.8 | 0.8 | 0.7 | 1.6 | 1.8 | 1.1 |
| AAVrh39 | 2.6 | 3.3 | 3.0 | 1.6 | 1.3 | 2.4 | 1.2 | 2.2 | 1.1 | 1.9 | 3.1 | 1.2 |
| AAVrh43 | 1.5 | 1.7 | 0.8 | 0.7 | 1.0 | 2.9 | 1.5 | 3.2 | 1.3 | 4.5 | 2.2 | 1.7 |
| AAVDJ | 1.5 | 1.1 | 5.6 | 0.2 | 1.3 | 4.9 | 5.0 | 16.9 | 0.8 | 1.6 | 3.5 | 1.0 |
| AAVDJ8 | 0.4 | 0.4 | 0.6 | 0.1 | 0.4 | 0.8 | 0.9 | 2.9 | 0.6 | 0.4 | 0.4 | 0.4 |
| Pig | 0.2 | 0.2 | 0.5 | 0.1 | 0.2 | 0.4 | 0.4 | 0.6 | 0.2 | 0.3 | 0.3 | 0.2 |
| Mouse | 0.0 | 0.0 | 0.0 | 0.1 | 0.0 | 0.0 | 0.0 | 0.0 | 0.0 | 0.0 | 0.0 | 0.0 |
| Avian | 0.0 | 0.0 | 0.0 | 0.0 | 0.0 | 0.1 | 0.0 | 0.0 | 0.0 | 0.0 | 0.0 | 0.0 |

The DNA-barcode results (Tables 6 and 7) show that AAV11, AAV11mt1, AAV2 AAV2mt2, AAV2mt3, AAV2mt4, AAV2mt5, AAV2mt6, AAV2mt7, AAV2mt8, AAV2mt9, AAV2mt1, AAV6mt2, AAV6mt4, AAV6mt5, AAV8, AAV9mt1, AAV9mt6, AAV11, AAVrh10, AAVrh39, AAVDJ and pig provide >7.5-fold greater biodistribution to multiple regions of the CNS such as frontal gyrus occipital cortex, caudate, hippocampus, cingulate gyrus, thalamus, hypothalamus, pons, medulla, cerebellar Purkinje layer, and cerebellar granular layer. For the putamen, AAV9mt6 provided 24.2-fold (Table 6) and 20.5-fold (Table 7) higher biodistribution relative to AAV9. For the caudate, AAV2mt2 and AAV2 showed the highest biodistribution—180.6 and 143.6-fold that of AAV9, respectively for animal #1 (Table 6), and 576.1 and 570.1-fold that of AAV9, respectively for animal #2 (Table 7). For the hippocampus, AAV11 and AAVDJ provided the highest biodistribution—66.3 and 40.3-fold that of AAV9, respectively for animal #1 (Table 6), and 15.6 and 18.6-fold that of AAV9, respectively for animal #2 (Table 7). Similarly, for cingulate gyrus AAV11 and AAVDJ provided the highest biodistribution—64.9 and 51.8-fold that of AAV9, respectively, for animal #1 (Table 6), and 220.3 and 91.9-fold that of AAV9, respectively, for animal #2 (Table 7). For pons and medulla, AAV2mt2, AAV2mt5, AAV2mt7, AAV2mt8 and AAVDJ provided the highest biodistribution—12 to 335-fold that of AAV9 (Tables 6 and 7).

The RNA-barcode results (Table 8 and 9) show that AAV1, AAV3B, AAV3mt4, AAV6, AAV6mt1, and AAV6mt3 provided better RNA expression than AAV9 consistently across both animals in particular CNS regions. AAV1 showed higher RNA expression than AAV9 in caudate, thalamus and hypothalamus in both animals (Tables 8 and 9). AAV3B and AAV3mt4 provided higher RNA expression than AAV9 in pons, medulla and cerebellar cortex in both animals (Tables 8 and 9). AAV6, AAV6mt1, and AAV6mt3 provided higher RNA expression than AAV9 in caudate, hippocampus, thalamus and hypothalamus in both animals (Table 8 and Table 9).

These results support CM administration of AAV1, AAV6, AAV6mt1, or AAV6mt3 for delivery of a payload molecule, for example, a modulatory polynucleotide or transgene, to the caudate, for treatment of Huntington's Disease, and for treatment of other diseases involving the caudate.

These results support CM administration of AAV6, AAV6mt1, or AAV6mt3 for delivery of a payload molecule, for example, a modulatory polynucleotide or transgene, to the hippocampus, for treatment of Alzheimer's Disease, and for treatment of other diseases involving the hippocampus.

What is claimed is:

1. A method of delivering a payload molecule to a brain region of a subject, comprising administering an AAV particle to cerebrospinal fluid (CSF) of the subject, wherein the AAV particle comprises a viral genome that encodes the payload molecule and a capsid protein, whereby the payload molecule is expressed in the brain region, and wherein the capsid protein serotype is selected from the group consisting of AAV1mt1, AAV1mt2, AAV1mt3, AAV2mt1, AAV2mt2, AAV2mt3, AAV2mt4, AAV2mt5, AAV2mt6, AAV2mt7, AAV2mt8, AAV2mt9, AAV2mt10, AAV3mt1, AAV3mt2, AAV3mt3, AAV3mt4, AAV5mt1, AAV5mt2, AAV5mt3, AAV5mt4, AAV5mt5, AAV6mt1, AAV6mt2, AAV6mt3, AAV6mt4, AAV6mt5, AAV9mt1, AAV9mt2, AAV9mt3, AAV9mt4, AAV9mt5, AAV9mt6, AAV9mt7, AAV9mt8, AAV9mt9, AAV9mt10, AAV9mt11, AAV9mt12, AAV11, AAVrh8, AAVrh10, AAVrh39, AAVrh43, AAVDJ, and AAVDJ8.

2. The method of claim 1, wherein the route of administration of the AAV particle is intrathecal (IT).

3. The method of claim 1, wherein the route of administration of the AAV particle is intracerebroventricular (ICV).

4. The method of claim 1, wherein the route of administration of the AAV particle is cisterna magna (CM).

5. The method of any one of claims 1-4, wherein the brain region is selected from the group consisting of frontal cortex, occipital cortex, caudate nucleus, putamen, thalamus, hippocampus, cingulate gyrus, hypothalamus, pons, medulla, cerebellar Purkinje layer, and cerebellar granular layer.

6. A method of delivering a payload molecule to a brain region of a subject, comprising administering an AAV particle to cerebrospinal fluid (CSF) of the subject, wherein the AAV particle comprises a viral genome that encodes the payload molecule and a capsid protein, whereby the payload molecule is expressed in the brain region, and wherein the brain region is caudate and the capsid protein serotype is selected from the group consisting of AAV1, AAV6, AAV6mt1, and AAV6mt3.

7. The method of claim 6, whereby the capsid protein is AAV6.

8. A method of delivering a payload molecule to a brain region of a subject, comprising administering an AAV particle to cerebrospinal fluid (CSF) of the subject, wherein the AAV particle comprises a viral genome that encodes the payload molecule and a capsid protein, whereby the payload molecule is expressed in the brain region, and wherein the brain region is selected from the group consisting of caudate, thalamus, and hippocampus and the capsid protein serotype is selected from the group consisting of AAV6, AAV6mt1, and AAV6mt3.

9. The method of claim 8, wherein the brain region is hippocampus.

10. The method of claim 8, wherein the brain region is thalamus.

11. A method of delivering a payload molecule to a brain region of a subject, comprising administering an AAV vector to cerebrospinal fluid (CSF) of the subject, wherein the AAV vector comprises a viral genome that encodes the payload molecule and a capsid protein, whereby the payload molecule is expressed in the brain region, and wherein the brain region is the caudate, thalamus and/or hypothalamus region and the capsid protein serotype is AAV1.

12. A method of delivering a payload molecule to a brain region of a subject, comprising administering an AAV vector to cerebrospinal fluid (CSF) of the subject, wherein the AAV vector comprises a viral genome that encodes the payload molecule and a capsid protein, whereby the payload molecule is expressed in the brain region, and wherein the brain region is the pons, medulla, and/or cerebellar cortex region and the capsid protein serotype is AAV3B or AAV3mt4.

13. A method of delivering a payload molecule to a brain region of a subject, comprising administering an AAV particle to cerebrospinal fluid (CSF) of the subject, wherein the AAV particle comprises a viral genome that encodes the payload molecule and a capsid protein, whereby the payload molecule is expressed in the brain region, and wherein the AAV particle shows at least 10-fold higher distribution in the brain region than AAV9 particle.

14. A method of delivering a payload molecule to a brain region of a subject, comprising administering an AAV particle to cerebrospinal fluid (CSF) of the subject, wherein the AAV particle comprises a viral genome that encodes the payload molecule and a capsid protein, whereby the payload molecule is expressed in the brain region, and wherein the AAV particle shows at least 20-fold higher distribution in the brain region than AAV9 particle.

15. A method of delivering a payload molecule to a brain region of a subject, comprising administering an AAV particle to cerebrospinal fluid (CSF) of the subject, wherein the AAV particle comprises a viral genome that encodes the payload molecule and a capsid protein, whereby the payload molecule is expressed in the brain region, and wherein the AAV particle shows at least 50-fold higher distribution in the brain region than AAV9 particle.

16. The method of any one of claims 13-15, wherein the brain region is frontal gyrus and the capsid protein serotype is selected from the group consisting of AAV1, AAV1mt1, AAV2mt8, AAV6mt2, AAV6mt4, AAV6mt5, AAV8, AAV11, AAVrh10, AAVrh39, and AAVDJ.

17. The method of any one of claims 13-15, wherein the brain region is occipital cortex and the capsid protein serotype is selected from the group consisting of AAV1, AAV1mt1, AAV6mt2, AAV6mt4, AAV6mt5, AAV8, AAV11, AAVrh10, AAVrh39, and AAVDJ.

18. The method of any one of claims 13-15, wherein the brain region is caudate, and the capsid protein serotype is selected from the group consisting of AAV1, AAV1mt1, AAV2, AAV2mt2, AAV2mt3, AAV2mt4, AAV2mt5, AAV2mt6, AAV2mt7, AAV2mt8, AAV2mt9, AAV2mt10, AAV6, AAV6mt1, AAV6mt2, AAV6mt3, AAV6mt4, AAV6mt5, AAV9mt1, AAV9mt6, and AAVDJ.

19. The method of any one of claims 13-15, wherein the brain region is putamen, and the capsid protein serotype is AAV9mt6.

20. The method of any one of claims 13-15, wherein the brain region is hippocampus, and the capsid protein serotype is selected from the group consisting of AAV1, AAV1mt1, AAV2, AAV2mt2, AAV2mt5, AAV2mt6, AAV2mt7, AAV2mt8, AAV2mt9, AAV2mt10, AAV6, AAV6mt1, AAV6mt2, AAV6mt3, AAV6mt4, AAV6mt5, AAV9mt1, AAV9mt6, AAV11, and AAVDJ.

21. The method of any one of claims 13-15, wherein the brain region is cingulate gyrus, and the capsid protein serotype is selected from the group consisting of AAV1, AAV1mt1, AAV2, AAV2mt2, AAV2mt4, AAV2mt5, AAV2mt6, AAV2mt7, AAV2mt8, AAV2mt9, AAV2mt10, AAV3B, AAV3mt1, AAV3mt2, AAV3mt3, AAV3mt4, AAV6, AAV6mt1, AAV6mt2, AAV6mt4, AAV6mt5, AAV9mt1, AAV11, AAVrh39, AAVDJ, and Pig.

22. The method of any one of claims 13-15, wherein the brain region is thalamus, and the capsid protein serotype is selected from the group consisting of AAV1, AAV1mt1, AAV2, AAV2mt2, AAV2mt9, AAV4, AAV6, AAV6mt1, AAV6mt2, AAV6mt3, AAV6mt4, AAV6mt5, AAV9mt3, and AAV9mt6.

23. The method of any one of claims 13-15, wherein the brain region is hypothalamus, and the capsid protein serotype is selected from the group consisting of AAV1, AAV1mt1, AAV2, AAV2mt2, AAV2mt3, AAV2mt4, AAV2mt5, AAV2mt6, AAV2mt7, AAV2mt8, AAV2mt9, AAV2mt10, AAV3B, AAV3mt1, AAV3mt2, AAV3mt3, AAV3mt4, AAV6mt2, AAV6mt4, AAV6mt5, AAV9mt1, AAV9mt6, AAV11, and AAVDJ.

24. The method of any one of claims 13-15, wherein the brain region is pons, and the capsid protein serotype is selected from the group consisting of AAV1, AAV1mt1, AAV2, AAV2mt2, AAV2mt4, AAV2mt5, AAV2mt6, AAV2mt7, AAV2mt8, AAV2mt10, AAV6mt4, AAV6mt5, AAV9mt1, AAV9mt6, AAV11, and AAVDJ.

25. The method of any one of claims 13-15, wherein the brain region is medulla, and the capsid protein serotype is selected from the group consisting of AAV1, AAV1mt1, AAV2, AAV2mt2, AAV2mt3, AAV2mt4, AAV2mt5, AAV2mt6, AAV2mt7, AAV2mt8, AAV2mt9, AAV2mt10, AAV3B, AAV3mt1, AAV3mt2, AAV3mt3, AAV3mt4, AAV6mt2, AAV6mt4, AAV6mt5, AAV9mt1, AAV11, AAVrh39, and AAVDJ.

26. The method of any one of claims 13-15, wherein the brain region is cerebellar Purkinje layer, and the capsid protein serotype is selected from the group consisting of AAV1, AAV1mt1, AAV2, AAV2mt2, AAV2mt5, AAV2mt6, AAV2mt7, AAV2mt8, AAV2mt9, AAV2mt10, AAV6mt2, AAV6mt4, AAV6mt5, AAV8, AAV9mt1, AAV11, AAVrh10, AAVrh39, and AAVDJ.

27. The method of any one of claims 13-15 wherein the brain region is cerebellar Granular layer, and the capsid protein serotype is selected from the group consisting of AAV1, AAV1mt1, AAV2, AAV2mt2, AAV2mt5, AAV2mt6, AAV2mt7, AAV2mt8, AAV2mt9, AAV2mt10, AAV6, AAV6mt1, AAV6mt2, AAV6mt3, AAV6mt4, AAV6mt5, AAV8, AAV9mt1, AAV9mt6, AAV11, AAVrh10, AAVrh39, and AAVDJ.

28. The method of any one of claims 13-27, whereby the distribution in the brain is measured by DNA bar coding.

29. A method of delivering a payload molecule to a brain region of a subject, comprising administering an AAV particle to cerebrospinal fluid (CSF) of the subject, wherein the AAV particle comprises a viral genome that encodes the payload molecule and a capsid protein, whereby the payload molecule is expressed in the brain region, and wherein the AAV particle shows at least 10-fold higher expression in the brain region than AAV9 particle.

30. A method of delivering a payload molecule to a brain region of a subject, comprising administering an AAV particle to cerebrospinal fluid (CSF) of the subject, wherein the AAV particle comprises a viral genome that encodes the payload molecule and a capsid protein, whereby the payload molecule is expressed in the brain region, and wherein the AAV particle shows at least 20-fold higher expression in the brain region than AAV9 particle.

31. A method of delivering a payload molecule to a brain region of a subject, comprising administering an AAV particle to cerebrospinal fluid (CSF) of the subject, wherein the AAV particle comprises a viral genome that encodes the payload molecule and a capsid protein, whereby the payload molecule is expressed in the brain region, and wherein the AAV particle shows at least 50-fold higher expression in the brain region than AAV9 particle.

32. The method of any one of claims 29-31, wherein the brain region is frontal gyrus and the capsid protein serotype is selected from the group consisting of AAV1, AAV1mt1, AAV2mt8, AAV6mt2, AAV6mt4, AAV6mt5, AAV8, AAV11, AAVrh10, AAVrh39, and AAVDJ.

33. The method of any one of claims 29-31, wherein the brain region is caudate, and the capsid protein serotype is selected from the group consisting of AAV1, AAV1mt1, AAV2, AAV2mt2, AAV2mt10, AAV6, and AAV6mt1.

34. The method of any one of claims 29-31, wherein the brain region is hippocampus, and the capsid protein serotype is selected from the group consisting of AAV6, AAV6mt1, and AAV6mt3.

35. The method of any one of claims 29-31, wherein the brain region is thalamus, and the capsid protein serotype is selected from the group consisting of AAV1, AAV2, AAV2mt2, AAV6, AAV6mt1, AAV6mt3, AAV6mt5, AAV9mt6.

36. The method of any one of claims 29-31, wherein the brain region is hypothalamus, and the capsid protein serotype is selected from the group consisting of AAV2, AAV2mt2, AAV2mt5, AAV2mt9, AAV9mt6, and AAVDJ.

37. The method of any one of claims 29-31, wherein the brain region is pons, and the capsid protein serotype is selected from the group consisting of AAV3B and AAV3mt4.

38. The method of any one of claims 29-31, wherein the brain region is medulla, and the capsid protein serotype is selected from the group consisting of AAV3B and AAV3mt4.

39. The method of any one of claims 29-31, wherein the brain region is cerebellar Purkinje layer, and the capsid protein serotype is selected from the group consisting of AAV3B and AAV3mt4.

40. The method of any one of claims 29-31, wherein the brain region is cerebellar Granular layer, and the capsid protein serotype is selected from the group consisting of AAV6 and AAV6mt1.

41. The method of any one of claims 29-31, wherein the brain region is caudate, and the capsid protein is AAV6.

42. The method of any one of claims 29-31, wherein the brain region is thalamus, and the capsid protein is selected from the group consisting of AAV6, AAV6mt1, and AAV6mt3.

43. The method of any one of claims 29-42, whereby expression in the brain region is measured by RNA bar coding.

44. The method of any one of claims 1-43, wherein the payload molecule is a polynucleotide.

45. The method of claim 44, wherein the polynucleotide is an siRNA duplex.

46. The method of claim 45, wherein the siRNA duplex, when expressed inhibits or suppresses the expression of a gene of interest in a cell.

47. The method of claim 46, wherein the gene of interest is selected from the group consisting of superoxide dismutase 1 (SOD1), chromosome 9 open reading frame 72 (C9ORF72), TAR DNA binding protein (TARDBP), ataxin 3 (ATXN3), huntingtin (HTT), amyloid precursor protein (APP), apolipoprotein E (APOE), microtubule-associated protein tau (MAPT), alpha synuclein (SNCA), voltage-gated sodium channel alpha subunit 9 (SCN9A), and voltage-gated sodium channel alpha subunit 10 (SCN10A).

48. The method of any one of claims 1-43, wherein the payload molecule is a polypeptide.

49. The method of claim 48, wherein the polypeptide is selected from the group consisting of Aromatic L-Amino Acid Decarboxylase (AADC), APOE2, Frataxin, survival motor neuron (SMN) protein, glucocerebrosidase (GCase), N-sulfoglucosamine sulfohydrolase, N-acetyl-alpha-glucosaminidase, iduronate 2-sulfatase, alpha-L-iduronidase, palmitoyl-protein thioesterase 1, tripeptidyl peptidase 1, battenin, CLN5, CLN6 (linclin), MFSD8, CLN8, aspartoacylase (ASPA), progranulin (GRN), MeCP2, beta-galactosidase (GLB1), gigaxonin (GAN), ATPase Sarcoplasmic/Endoplasmic Reticulum Ca2+ Transporting 2 (ATP2A2), an antibody, and S100 Calcium Binding Protein A1 (S100A1).

50. The method of any one of claims 1-49, wherein the subject is a mammal.

51. The method of any one of claims 1-50, wherein the subject is a human.

52. The method of any of the claims 1-51, whereby the AAV particle is for treatment, amelioration, or prevention of a neurological disease.

53. The method of claim 52, wherein the neurological disease stems from a loss or partial loss of protein or function of a protein in the subject.

54. The method of claim 53, wherein the neurological disease is selected from the group consisting of Parkinson's Disease (PD), Multiple System Atrophy (MSA), and Friedreich's Ataxia (FA).

55. The method of claim 52, wherein the neurological disease stems from a gain or partial gain of function mutation in a protein in the subject.

56. The method of claim 55, wherein the neurological disease is selected from the group consisting of tauopathies, Alzheimer's disease (AD), Amyotrophic lateral sclerosis (ALS), Huntington's Disease (HD), and neuropathic pain.

57. A method of treating Huntington's Disease in a subject comprising administering to the cerebrospinal fluid (CSF) of the subject an AAV particle comprising a viral genome that encodes a payload molecule and a capsid protein to a brain region of a subject with Huntington's Disease, wherein the route of administration is CM administration and whereby the payload molecule is expressed in the brain region, wherein the capsid protein is selected from the group consisting of AAV1, AAV6, AAV6mt1, and AAV6mt3, and wherein the payload molecule is a modulatory polynucleotide that suppresses or inhibits expression of HTT.

58. The method of claim 57, wherein the brain region is caudate.

59. The method of claim 57 or 58, wherein the modulatory polynucleotide is an siRNA duplex.

60. A method of treating Alzheimer's Disease in a subject comprising administering to the cerebrospinal fluid (CSF) of the subject an AAV particle comprising a viral genome that encodes a payload molecule and a capsid protein to a brain region of a subject with Alzheimer's Disease, wherein the route of administration is CM administration and whereby the payload molecule is expressed in the brain region, wherein the capsid protein is selected from the group consisting of AAV6, AAV6mt1, or AAV6mt3, and wherein the payload molecule is a modulatory polynucleotide that suppresses or inhibits expression of amyloid precursor protein, microtubule-associated protein tau, or alpha synuclein

61. The method of claim 60, wherein the brain region is hippocampus.

62. The method of claim 60 or 61, wherein the modulatory polynucleotide is an siRNA duplex.
